SEQUENCE LISTING (97 sequences)

SEQ ID NO: 1 - DNA, 20 bp, synthetic construct
  misc_feature 1..20: note = Alp primer forward
  source 1..20: mol_type = other DNA
ccagcaggtttctctcttgg

SEQ ID NO: 2 - DNA, 20 bp, synthetic construct
  misc_feature 1..20: note = Alp primer reverse
  source 1..20: mol_type = other DNA
gggatggaggagagaaggtc

SEQ ID NO: 3 - DNA, 20 bp, synthetic construct
  misc_feature 1..20: note = Col primer forward
  source 1..20: mol_type = other DNA
gagcggagagtactggatcg

SEQ ID NO: 4 - DNA, 20 bp, synthetic construct
  misc_feature 1..20: note = Col primer reverse
  source 1..20: mol_type = other DNA
gttcgggctgatgtaccagt

SEQ ID NO: 5 - DNA, 20 bp, synthetic construct
  misc_feature 1..20: note = Opn primer forward
  source 1..20: mol_type = other DNA
tctgatgagaccgtcactgc

SEQ ID NO: 6 - DNA, 20 bp, synthetic construct
  misc_feature 1..20: note = Opn primer reverse
  source 1..20: mol_type = other DNA
aggtcctcatctgtggcatc

SEQ ID NO: 7 - DNA, 20 bp, synthetic construct
  misc_feature 1..20: note = Runx 2 primer forward
  source 1..20: mol_type = other DNA
cccagccacctttacctaca

SEQ ID NO: 8 - DNA, 20 bp, synthetic construct
  misc_feature 1..20: note = Runx 2 primer reverse
  source 1..20: mol_type = other DNA
tatggagtgctgctggtctg

SEQ ID NO: 9 - DNA, 22 bp, synthetic construct
  misc_feature 1..22: note = Arg-1 primer forward
  source 1..22: mol_type = other DNA
ctccaagccaaagtccttagag

SEQ ID NO: 10 - DNA, 22 bp, synthetic construct
  misc_feature 1..22: note = Arg-1 primer reverse
  source 1..22: mol_type = other DNA
aggagctgtcattagggacatc

SEQ ID NO: 11 - DNA, 23 bp, synthetic construct
  misc_feature 1..23: note = Mgl-1 primer forward
  source 1..23: mol_type = other DNA
tgagaaaggctttaagaactggg

SEQ ID NO: 12 - DNA, 21 bp, synthetic construct
  misc_feature 1..21: note = Mgl-1 primer reverse
  source 1..21: mol_type = other DNA
gaccacctgtagtgatgtggg

SEQ ID NO: 13 - DNA, 19 bp, synthetic construct
  misc_feature 1..19: note = Cd206 primer forward
  source 1..19: mol_type = other DNA
ttccatcgagactgctgct

SEQ ID NO: 14 - DNA, 19 bp, synthetic construct
  misc_feature 1..19: note = Cd206 primer reverse
  source 1..19: mol_type = other DNA
ccagagggatcgcctgttt

SEQ ID NO: 15 - DNA, 21 bp, synthetic construct
  misc_feature 1..21: note = Ym1 primer forward
  source 1..21: mol_type = other DNA
catgagcaagacttgcgtgac

SEQ ID NO: 16 - DNA, 21 bp, synthetic construct
  misc_feature 1..21: note = Ym1 primer reverse
  source 1..21: mol_type = other DNA
ggtccaaacttccatcctcca

SEQ ID NO: 17 - DNA, 19 bp, synthetic construct
  misc_feature 1..19: note = Socs3primer forward
  source 1..19: mol_type = other DNA
gatttcgcttcgggactag

SEQ ID NO: 18 - DNA, 20 bp, synthetic construct
  misc_feature 1..20: note = Socs3 primer reverse
  source 1..20: mol_type = other DNA
cggcggcgggaaacttgctg

SEQ ID NO: 19 - DNA, 23 bp, synthetic construct
  misc_feature 1..23: note = Tnfalpha primer forward
  source 1..23: mol_type = other DNA
tctcagcctcttctcattcctgc

SEQ ID NO: 20 - DNA, 23 bp, synthetic construct
  misc_feature 1..23: note = Tnfalpha primer reverse
  source 1..23: mol_type = other DNA
agaactgatgagagggaggccat

SEQ ID NO: 21 - DNA, 20 bp, synthetic construct
  misc_feature 1..20: note = iNOS primer forward
  source 1..20: mol_type = other DNA
aatcttggagcgagttgtgg

SEQ ID NO: 22 - DNA, 21 bp, synthetic construct
  misc_feature 1..21: note = iNOS primer reverse
  source 1..21: mol_type = other DNA
caggaagtaggtgagggcttg

SEQ ID NO: 23 - DNA, 24 bp, synthetic construct
  misc_feature 1..24: note = IL-6 primer forward
  source 1..24: mol_type = other DNA
acaaagccagagtccttcagagag

SEQ ID NO: 24 - DNA, 24 bp, synthetic construct
  misc_feature 1..24: note = IL-6 primer reverse
  source 1..24: mol_type = other DNA
ttggatggtcttggtccttagcca

SEQ ID NO: 25 - DNA, 20 bp, synthetic construct
  misc_feature 1..20: note = IL-1beta primer forward
  source 1..20: mol_type = other DNA
agagcttcaggcaggcagta

SEQ ID NO: 26 - DNA, 20 bp, synthetic construct
  misc_feature 1..20: note = IL-1beta primer reverse
  source 1..20: mol_type = other DNA
aggtgctcatgtcctcatcc

SEQ ID NO: 27 - DNA, 20 bp, synthetic construct
  misc_feature 1..20: note = Mcp-1 primer forward
  source 1..20: mol_type = other DNA
ccagcaagatgatcccaatg

SEQ ID NO: 28 - DNA, 20 bp, synthetic construct
  misc_feature 1..20: note = Mcp-1 primer reverse
  source 1..20: mol_type = other DNA
ttcttggggtcagcacagac

SEQ ID NO: 29 - DNA, 22 bp, synthetic construct
  misc_feature 1..22: note = Prkaa1 primer forward
  source 1..22: mol_type = other DNA
ggtgtacggaaggcaaaatggc

SEQ ID NO: 30 - DNA, 23 bp, synthetic construct
  misc_feature 1..23: note = Prkaa1 primer reverse
  source 1..23: mol_type = other DNA
caggattcttccttcgtacacgc

SEQ ID NO: 31 - DNA, 22 bp, synthetic construct
  misc_feature 1..22: note = Prkaa2 primer forward
  source 1..22: mol_type = other DNA
ctgaagccagagaatgtgctgc

SEQ ID NO: 32 - DNA, 22 bp, synthetic construct
  misc_feature 1..22: note = Prkaa2 primer reverse
  source 1..22: mol_type = other DNA
gagatgacctcaggtgctgcat

SEQ ID NO: 33 - DNA, 23 bp, synthetic construct
  misc_feature 1..23: note = Prkab1 primer forward
  source 1..23: mol_type = other DNA
ccaaaagtgctccgatgtgtctg

SEQ ID NO: 34 - DNA, 22 bp, synthetic construct
  misc_feature 1..22: note = Prkab1 primer reverse
  source 1..22: mol_type = other DNA
gggctttgaacctctcttctgg

SEQ ID NO: 35 - DNA, 22 bp, synthetic construct
  misc_feature 1..22: note = Prkab2 primer forward
  source 1..22: mol_type = other DNA
gacttcgttgccatcctggatc

SEQ ID NO: 36 - DNA, 22 bp, synthetic construct
  misc_feature 1..22: note = Prkab2 primer reverse
  source 1..22: mol_type = other DNA
ccaagctgactggtaaccacag

SEQ ID NO: 37 - DNA, 22 bp, synthetic construct
  misc_feature 1..22: note = Prkag1 primer forward
  source 1..22: mol_type = other DNA
tctccgccttacctgtagtgga

SEQ ID NO: 38 - DNA, 22 bp, synthetic construct
  misc_feature 1..22: note = Prkag1 primer reverse
  source 1..22: mol_type = other DNA
gcagggcttttgtcacagacac

SEQ ID NO: 39 - DNA, 23 bp, synthetic construct
  misc_feature 1..23: note = Prkag2 primer forward
  source 1..23: mol_type = other DNA
ctcctcatccaaagagtcttcgc

SEQ ID NO: 40 - DNA, 22 bp, synthetic construct
  misc_feature 1..22: note = Prkag2 primer reverse
  source 1..22: mol_type = other DNA
tgggtgttgacggagaagagga

SEQ ID NO: 41 - DNA, 22 bp, synthetic construct
  misc_feature 1..22: note = Prkag3 primer forward
  source 1..22: mol_type = other DNA
aagcggctactcaagttcctgc

SEQ ID NO: 42 - DNA, 23 bp, synthetic construct
  misc_feature 1..23: note = Prkag3 primer reverse
  source 1..23: mol_type = other DNA
ccagaactacagccaaatctcgg

SEQ ID NO: 43 - DNA, 23 bp, synthetic construct
  misc_feature 1..23: note = Gapdh primer forward
  source 1..23: mol_type = other DNA
catcactgccacccagaagactg

SEQ ID NO: 44 - DNA, 23 bp, synthetic construct
  misc_feature 1..23: note = Gapdh primer reverse
  source 1..23: mol_type = other DNA
atgccagtgagcttcccgttcag

SEQ ID NO: 45 - DNA, 24 bp, synthetic construct
  misc_feature 1..24: note = Bglap1 primer forward
  source 1..24: mol_type = other DNA
gcaataaggtagtgaacagactcc

SEQ ID NO: 46 - DNA, 22 bp, synthetic construct
  misc_feature 1..22: note = Bglap1 primer reverse
  source 1..22: mol_type = other DNA
ccatagatgcgtttgtaggcgg

SEQ ID NO: 47 - DNA, 24 bp, synthetic construct
  misc_feature 1..24: note = Bglap2 primer forward
  source 1..24: mol_type = other DNA
gcaataaggtagtgaacagactcc

SEQ ID NO: 48 - DNA, 22 bp, synthetic construct
  misc_feature 1..22: note = Bglap2 primer reverse
  source 1..22: mol_type = other DNA
gcgtttgtaggcggtcttcaag

SEQ ID NO: 49 - DNA, 22 bp, synthetic construct
  misc_feature 1..22: note = Got1(Ast) primer forward
  source 1..22: mol_type = other DNA
tgctactgggatgcggagaaga

SEQ ID NO: 50 - DNA, 22 bp, synthetic construct
  misc_feature 1..22: note = Got1(Ast) primer reverse
  source 1..22: mol_type = other DNA
tgcatgacagcagcgatctgct

SEQ ID NO: 51 - DNA, 22 bp, synthetic construct
  misc_feature 1..22: note = Gpt1(Alt) primer forward
  source 1..22: mol_type = other DNA
ccactcagtctctaagggctac

SEQ ID NO: 52 - DNA, 22 bp, synthetic construct
  misc_feature 1..22: note = Gpt1(Alt) primer reverse
  source 1..22: mol_type = other DNA
acacaaccgcacgctcatcagt

SEQ ID NO: 53 - DNA, 22 bp, synthetic construct
  misc_feature 1..22: note = Ckb (Creatine) primer forward
  source 1..22: mol_type = other DNA
gctcattgacgaccacttcctc

SEQ ID NO: 54 - DNA, 22 bp, synthetic construct
  misc_feature 1..22: note = Ckb (Creatine) primer reverse
  source 1..22: mol_type = other DNA
cctcctcgttaatccacaccag

SEQ ID NO: 55 - DNA, 22 bp, synthetic construct
  misc_feature 1..22: note = mTOR primer forward
  source 1..22: mol_type = other DNA
agaagggtctccaaggacgact

SEQ ID NO: 56 - DNA, 22 bp, synthetic construct
  misc_feature 1..22: note = mTOR primer reverse
  source 1..22: mol_type = other DNA
gcaggacacaaaggcagcattg

SEQ ID NO: 57 - DNA, 22 bp, synthetic construct
  misc_feature 1..22: note = Bmp2 primer forward
  source 1..22: mol_type = other DNA
aacaccgtgcgcagcttccatc

SEQ ID NO: 58 - DNA, 22 bp, synthetic construct
  misc_feature 1..22: note = Bmp2 primer reverse
  source 1..22: mol_type = other DNA
cggaagatctggagttctgcag

SEQ ID NO: 59 - DNA, 22 bp, synthetic construct
  misc_feature 1..22: note = Bmpr2 primer forward
  source 1..22: mol_type = other DNA
agagacccaagttcccagaagc

SEQ ID NO: 60 - DNA, 22 bp, synthetic construct
  misc_feature 1..22: note = Bmpr2 primer reverse
  source 1..22: mol_type = other DNA
tctcctcagcacactgtgcagt

SEQ ID NO: 61 - DNA, 22 bp, synthetic construct
  misc_feature 1..22: note = Bmp7 primer forward
  source 1..22: mol_type = other DNA
ggagcgatttgacaacgagacc

SEQ ID NO: 62 - DNA, 22 bp, synthetic construct
  misc_feature 1..22: note = Bmp7 primer reverse
  source 1..22: mol_type = other DNA
agtggttgctggtggctgtgat

SEQ ID NO: 63 - DNA, 22 bp, synthetic construct
  misc_feature 1..22: note = Tgfbr2 primer forward
  source 1..22: mol_type = other DNA
cctactctgtctgtggatgacc

SEQ ID NO: 64 - DNA, 22 bp, synthetic construct
  misc_feature 1..22: note = Tgfbr2 primer reverse
  source 1..22: mol_type = other DNA
gacatccgtcgtcttgaacgac

SEQ ID NO: 65 - DNA, 20 bp, synthetic construct
  misc_feature 1..20: note = ALP primer forward
  source 1..20: mol_type = other DNA
atgggatgggtgtctccaca

SEQ ID NO: 66 - DNA, 19 bp, synthetic construct
  misc_feature 1..19: note = ALP primer reverse
  source 1..19: mol_type = other DNA
ccacgaaggggaacttgtc

SEQ ID NO: 67 - DNA, 20 bp, synthetic construct
  misc_feature 1..20: note = COL1A primer forward
  source 1..20: mol_type = other DNA
atgactatgagtgggaagca

SEQ ID NO: 68 - DNA, 21 bp, synthetic construct
  misc_feature 1..21: note = COL1A primer reverse
  source 1..21: mol_type = other DNA
tgggtccctctgttacacttt

SEQ ID NO: 69 - DNA, 18 bp, synthetic construct
  misc_feature 1..18: note = OPN primer forward
  source 1..18: mol_type = other DNA
ctcaggccagttgcagcc

SEQ ID NO: 70 - DNA, 24 bp, synthetic construct
  misc_feature 1..24: note = OPN primer reverse
  source 1..24: mol_type = other DNA
caaaagcaaatcactgcaattctc

SEQ ID NO: 71 - DNA, 20 bp, synthetic construct
  misc_feature 1..20: note = RUNX2 primer forward
  source 1..20: mol_type = other DNA
cctgaactctgcaccaagtc

SEQ ID NO: 72 - DNA, 20 bp, synthetic construct
  misc_feature 1..20: note = RUNX2 primer reverse
  source 1..20: mol_type = other DNA
gaggtggcagtgtcatcatc

SEQ ID NO: 73 - DNA, 21 bp, synthetic construct
  misc_feature 1..21: note = GAPDH primer forward
  source 1..21: mol_type = other DNA
ggcatccactgtggtcatgag

SEQ ID NO: 74 - DNA, 20 bp, synthetic construct
  misc_feature 1..20: note = GAPDH primer reverse
  source 1..20: mol_type = other DNA
tgcaccaccaactgcttagc

SEQ ID NO: 75 - DNA, 22 bp, synthetic construct
  misc_feature 1..22: note = 4EBP1 primer forward
  source 1..22: mol_type = other DNA
caccagcccttccagtgatgag

SEQ ID NO: 76 - DNA, 22 bp, synthetic construct
  misc_feature 1..22: note = 4EBP1 primer reverse
  source 1..22: mol_type = other DNA
ccttggtagtgctccacacgat

SEQ ID NO: 77 - DNA, 22 bp, synthetic construct
  misc_feature 1..22: note = AKT1 primer forward
  source 1..22: mol_type = other DNA
tggactacctgcactcggagaa

SEQ ID NO: 78 - DNA, 22 bp, synthetic construct
  misc_feature 1..22: note = AKT1 primer reverse
  source 1..22: mol_type = other DNA
gtgcggcaaaaggtcttcatgg

SEQ ID NO: 79 - DNA, 22 bp, synthetic construct
  misc_feature 1..22: note = S6K1 primer forward
  source 1..22: mol_type = other DNA
tattggcagcccacgaacacct

SEQ ID NO: 80 - DNA, 23 bp, synthetic construct
  misc_feature 1..23: note = S6K1 primer reverse
  source 1..23: mol_type = other DNA
gtcacatccatctgctctatgcc

SEQ ID NO: 81 - protein, 7 aa, synthetic construct
  REGION 1..7: note = HKUOT-S2 peptide
KTVSLPR

SEQ ID NO: 82 - protein, 18 aa, synthetic construct
  REGION 1..18: note = HKUOT-S2 peptide
KGNLLECDGGNTAQMMAR

SEQ ID NO: 83 - protein, 10 aa, synthetic construct
  REGION 1..10: note = HKUOT-S2 peptide
TKSSLPGQTK

SEQ ID NO: 84 - protein, 7 aa, synthetic construct
  REGION 1..7: note = HKUOT-S2 peptide
KEVSLPR

SEQ ID NO: 85 - protein, 8 aa, synthetic construct
  REGION 1..8: note = HKUOT-S2 peptide
IKITTYRQ

SEQ ID NO: 86 - protein, 19 aa, synthetic construct
  REGION 1..19: note = HKUOT-S2 peptide
AASECEEAGFSVCVEVNGR

SEQ ID NO: 87 - protein, 15 aa, synthetic construct
  REGION 1..15: note = HKUOT-S2 peptide
APSTYGGGLSVSSSR

SEQ ID NO: 88 - protein, 13 aa, synthetic construct
  REGION 1..13: note = HKUOT-S2 peptide
DDSITPTEDSIKR

SEQ ID NO: 89 - protein, 40 aa, synthetic construct
  REGION 1..40: note = HKUOT-S2 peptide
DTANLFPQTSLSLFMKPDTAGTFDVECLTTDHYTGGMKQK

SEQ ID NO: 90 - protein, 10 aa, synthetic construct
  REGION 1..10: note = HKUOT-S2 peptide
FKLLNYCIPK

SEQ ID NO: 91 - protein, 18 aa, synthetic construct
  REGION 1..18: note = HKUOT-S2 peptide
HAETSSGGQAASSHEQAR

SEQ ID NO: 92 - protein, 30 aa, synthetic construct
  REGION 1..30: note = HKUOT-S2 peptide
QADLILTAGTVTMKMAPSLVRLYEQMAEPK

SEQ ID NO: 93 - protein, 11 aa, synthetic construct
  REGION 1..11: note = HKUOT-S2 peptide
RSKLGSHHIDR

SEQ ID NO: 94 - protein, 8 aa, synthetic construct
  REGION 1..8: note = HKUOT-S2 peptide
SISISVAR

SEQ ID NO: 95 - protein, 9 aa, synthetic construct
  REGION 1..9: note = HKUOT-S2 peptide
SLVNLGGSK

SEQ ID NO: 96 - protein, 20 aa, synthetic construct
  REGION 1..20: note = HKUOT-S2 peptide
TEDGSDPPSGDFLTEGGGVR

SEQ ID NO: 97 - protein, 8 aa, synthetic construct
  REGION 1..8: note = HKUOT-S2 peptide
VATVSLPR

(19) United States
(12) Patent Application Publication
(10) Pub. No.: US 2026/0049109 A1
(43) Pub. Date: Feb. 19, 2026

(54) NOVEL IMMUNOMODULATORY, NEUROMODULATORY, OSTEOGENIC, AND ANTI-OSTEOPOROTIC HKUOT-S2 PROTEIN THAT ENHANCES BONE FRACTURE REPAIRS AND SUPPRESSES OSTEOPOROSIS DEVELOPMENT

(71) Applicant: The University of Hong Kong, Hong Kong (HK)

(72) Inventors: John Akrofi KUBI, Hong Kong (HK); Wai-Kwok Kelvin YEUNG, Hong Kong (HK); Augustine Suurinobah BRAH, Hong Kong (HK); Kenneth Man Chee CHEUNG, Hong Kong (HK)

(21) Appl. No.: 19/101,581

(22) PCT Filed: Aug. 10, 2023
(86) PCT No.: PCT/CN2023/112211

§ 371 Date: Feb. 5, 2025

Related U.S. Application Data
(60) Provisional application No. 63/371,229, filed on Aug. 12, 2022.

(51) Int. Cl. C07K 14/415; A61K 36/8945; A61K 38/16; A61P 3/10; A61P 19/00; A61P 19/10; A61P 29/00

(52) U.S. Cl. CPC C07K 14/415; A61K 36/8945; A61P 3/10; A61P 19/00; A61P 19/10; A61P 29/00; A61K 38/168

(57) ABSTRACT

Provided are compositions comprising the HKUOT-S2 protein, at least one peptide according to at least one of SEQ ID NO: 81-97, or proteins comprising an amino acid sequence according to at least one of SEQ ID NO: 81-97. Provided are methods of osteogenesis, including, for example, enhancing bone repair, macrophage polarizations that regulate pro-inflammatory and anti-inflammatory activities, and bone resorption by osteoclasts and methods of treating inflammatory diseases, spinal cord injuries, liver and kidney diseases, cardiovascular diseases, diabetes, post-menopausal syndrome, infertility, or hematopoietic diseases or enhancing wound healing.

### CROSS-REFERENCE TO RELATED APPLICATION

This application is a 371 application of the International Patent Application No. PCT/CN2023/112211 filed on Aug. 10, 2023, which claims priority from U.S. Provisional Patent Application Ser. No. 63/371,229 filed Aug. 12, 2022, and the disclosures of which are incorporated herein by reference in their entireties.

### REFERENCE TO SEQUENCE DISCLOSURE

The sequence listing file under the file name “UHK307XC1PCT-SeqList.xml” submitted in ST.26 XML file format with a file size of 120 KB created on Aug. 9, 2023 is incorporated herein by reference.

### BACKGROUND OF THE INVENTION

The incidence and prevalence of bone defects are global challenges that have severe public health and socioeconomic impacts on the population and health authorities. ^{[1-3]}Bone fracture risks increase with age-related medical conditions such as osteoporosis and bone fragility leading to frequent bone fractures, chronic pains and hospitalization. ^{[4]}It is projected that by 2050, more than half of global osteoporotic associated hip fractures, representing 3250 million cases, will predominantly occur in Asia. ^{[5]}It has also been estimated that the US government spends over $5 billion annually to provide medical interventions for treating various bone fractures. ^{[6]}However, some of the standard bone fracture treatment protocols, such as autologous bone grafts, have been reported to have clinical drawbacks, such as postsurgical donor site morbidity and microbial infections. ^{[6]}Consequently, application of tissue engineering and cell-based therapies for bone fracture treatments have been suggested to minimize some of the clinical drawbacks of these autografts and allografts bone fracture treatments. ^{[6]}It is, however, very challenging to find osteogenic stimuli that can safely trigger bone fracture healing with minimal side effects. Understanding the key molecular and cellular regulatory apparatuses during bone injuries and regeneration is essential to tissue engineering and cell-based therapies.^{[7, 8]}

Bone injury stimuli trigger cellular and molecular mechanisms that modulate sequence of events in well-orchestrated manner to progressively restore impaired bone integrity. ^{[9]}During bone injury, blood flow to the injury site is disrupted and recruited platelets function to aid blood clotting. ^{[10]}Neutrophils are the first immune cells that infiltrate the injury sites to perform phagocytotic, and anti-microbial functions. ^{[11]}Activities of the neutrophils and other cells trigger M1 and M2 macrophage-mediated inflammatory and anti-inflammatory responses, respectively in sequential manner. All these complex cellular events help to stimulate mesenchymal stem cells (MSCs)-osteoblasts differentiation. ^{[9, 12]}Molecules such as bone morphogenetic proteins (BMPs) stimulate MSCs-osteoblast differentiation during bone fracture repairs. ^{[13-16]}The entire bone fracture repair process can therefore be grouped into inflammatory, repair, and remodeling stages. ^{[9, 17]}Bone remodeling involves tightly regulated bone resorption by osteoclasts and bone formation by osteoblasts. ^{[18]}A homeostatic imbalance among these key cellular and molecular functions during inflammatory, repair, or remodeling processes could compromise physiological and architectural bone integrity, thereby increasing the risks of recurrent bone fractures. ^{[18]}Homeostatic imbalance between osteoclasts and osteoblasts activities during bone remodeling results in bone pathologies, such as osteoporosis. ^{[19]}Bone fracture repair induction is therefore very challenging for both patients and orthopedic surgeons, as it is difficult to establish optimal treatment protocols to restore the physiological equilibrium between bone resorption and formation.

Cell-based therapy and tissue engineering technology advancements have strongly demonstrated crosstalk among the relevant cellular components to promote bone fracture repairs. ^{[20]}It has been demonstrated that polarized M2 macrophage enhanced MSC-osteoblast differentiation. ^{[21]}Alternatively, it was reported that MSCs promoted M2 macrophage polarization for tissue regeneration. ^{[22]}It was also shown that a co-culture of M1 macrophages and MSCs enhanced osteoblast biomineralization. ^{[23]}All this evidence showed that appropriate osteogenic stimuli and conditions are needed to modulate the key cellular and molecular components to enhance bone fracture repairs. Cell-based therapies therefore take advantage of crosstalk and homeostatic coordination among the key cellular and molecular components for therapeutic targets for bone regeneration. ^{[20, 24, 25]}Pro-inflammatory activities by M1 macrophages are predominant in ovariectomized (OVX)-induced osteoporotic mice due to continues stimulation of M1 macrophage activities and reduction in M2 macrophage anti-inflammatory activities. ^{[26, 27]}Consequently, osteoclast-mediated bone resorption outweighs osteoblast-mediated bone formation leading to poor bone integrity. ^{[27]}Most anti-osteoporotic drugs, such as bisphosphonates, suppress osteoclast activities. ^{[28]}The anti-osteoporotic drug, Strontium ranelate, enhances osteoblast-mediated bone formation while suppressing osteoclast-mediated bone resorption. ^{[29]}However, prolonged use of these osteoclast suppressive anabolic and catabolic anti-osteoporotic drugs could potentially impair the regulatory osteoblast-osteoclast crosstalk. Prolonged use of bisphosphonates in osteoporotic patients was associated with an increased risks of atypical femoral fractures (AFF). ^{[30]}In a randomized, controlled trial of osteoporotic Japanese women, it was reported that both teriparatide and alendronate treated osteoporotic patients experienced negative side effects, such as infections and gastrointestinal and musculoskeletal disorders. ^{[31]}There is a growing advocacy for developing interventions that could facilitate optimal physiological and homeostatic balance between bone resorption and formation machinery to improve bone functional and architectural integrity with minimal side effects.^{[32]}

Diosgenin from Dioscorea spp. enhanced brain functions in healthy human subjects and mouse model of Alzheimer disorder, ^{[33]}inhibited bacterial and clinical fungal growth, ^{[34]}and induced MC3T3-1E cells to osteoblasts differentiation. ^{[35]}Dioscorin from Dioscorea spp. reportedly stimulated immunomodulatory functions in the RAW264.7 cells and mice, ^{[36]}whereas dispo85E also induced osteogenesis. ^{[37]}Estrogenic protein, designated DOI, from Dioscorea spp. rescued osteoporosis. ^{[38]}However, application of these Dioscorea spp. proteins to modulate cellular components, such macrophage polarization and MSCs-osteoblast differentiation to enhance bone defect repairs and mechanisms of actions, have not been fully explored.

Therefore, there remains a need for effective methods of osteogenesis.

### BRIEF SUMMARY OF THE INVENTION

The subject invention pertains to compositions comprising the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97. The subject invention further pertains to methods of osteogenesis, including, for example, enhancing bone repair. In certain embodiments, the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97 can induce macrophage polarization and human MSCs (hMSCs)-derived osteoblasts mineralization. In certain embodiments, the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97 can promote human mesenchymal stem cells (hMSCs) to osteoblast differentiation by increasing oestrogen receptor α (ERα), oestrogen receptor β (ERβ), and ALP mRNA expression. In certain embodiments, the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97 can upregulate osteogenic gene expressions of ALP, COL1A1 and RUNX2. In certain embodiments, the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97 can modulate genes enriched in focal adhesion (Itgb2, Il16, Hck, Keap1, Fblim1, Epha2, Git1, Actn1, Sdc4, Lims1, Tns3, Klfl1, Vc1, Lcp1, Efs, Sla, Enah, Mapre2, Itgb8, Il1rl1, Ptprc, Rsu1, Inpp5e, Ubox5, Src, Cpne3, Tln1, Mtf2, Zyx, Vasp, Parva, Tnfsf13b, Dlc1, Irf2, Bcar1, Rexo2, Senp1, Phldb2, Clasp2, Cbl, Zfp384, Srcin1, Fes, Mapk3, Mapk1), AMPK (Pparg, Tsc2, Cd36, Cp, Gys1, Cpt1c, Akt3, Rps6kb1, Rab10, Ppp2r5e, Pfkp, Cab391, Pdpk1, Ppp2r5b, Fasn, Pfkfb4, Creb1, Stradb, Ppp2r5a, Rab14, Creb311, Pck1, Ccna2, Prkaa2, Pik3r3, Rheb, Mtor, Add1, Ulk1, Prkab1, Ppp2r1b, Pik3cb, Eef2k, Cab39, Scd1, Irs2, Pik3cd, Elavl1, Pik3r1, Ppp2r2d, Acacb, Ppp2r3a, Foxo1, Foxo3, Scd4, Prkaa1, 9430076C15Rik, Irs4, Irs1, Lep, Mlycd, Cpt1b), PI3K-Akt, (Ccnd2, Pdgfb, Col6a1, Col1a1, Rac1, Ccne1, Tsc2, Lamb1, Cdkn1b, Gys1, Il7r, Pgf, Prlr, Epor, Tek, Epha2, Bcl2l1, Pten, Csf1, Atf6b, Akt3, Lama4, Kitl, Sgk1, Myb, Ddit4, Mdm2, Col6a2, Rps6kb1, Pik3cg, Itgb3, Pik3r5, Ppp2r5e, Syk, Thbs4, Ptk2, Nr4a1, Cdkn1a, Thbs2, Vegfa, Pdpk1, Mlst8, Sos1, Pdgfrb, Csf1r, Ppp2r5b, Vegfb, Itgb8, Cdk2, Hras, Il6, Pdgfa, Sgk3, Creb1, Lamc1, Ppp2r5a, Itga4, Itgav, Creb3l1, Bcl2l11, Pck1, Nras, Ngf, Cd2, Il6ra, Efna1, Pdgfc, Ccne2, Tnc, Prkaa2, Jak1, Pik3r3, Csf3r, Hgf, Casp9, Rheb, Mtor, Pdgfra, Spp1, Ibsp, Ereg, Col1a2, Gnb2, Kras, Cd19, Fgfr2, Fgf15, Angpt2, Col4a1, Col4a2, Fgfr1, Rbl2, Lpar2, Pdgfd, Ppp2r1b, Itga11, Pik3cb, Gng11, Tnxb, Ccnd3, Sos2, Lpar3, Lpar1, Tlr4, Chad, Pik3cd, Magi2, Pik3r1, Ppp2r2d, Itga1, Reln, Ppp2r3a, Col6a6, Phlpp1, Magi1, Col6a3, F2r, Foxo3, Prkaa1, Lamb2, 9430076C15Rik, Fgfr3, Ghr, Irs1, Bcl2, Trp53, Kdr, Mapk3, Mapk1, Gng5, Itga10) mTOR (Mtor, Prkaa1, Prkaa2 and Tnfα), BMP (Pparg, Vsir, Ube2o, Fstl1, Smad7, Skil, Tbx20, Trim33, Crb2, Smad6, Nbl1, Dlx1, Fzd1, Sorl1, Sox11, Numa1) and TGF-β (Ltbp1, Tgfb1, Tgfbr1, Id3, Bmp7, E2f4, Rps6kb1, Id2, Id4, Zfyve16, Fst, Bmpr1a, Bmp1, Crebbp, Bambi, Tnf, Smad7, Acvr1c, Acvr1, Fbn1, Rbl1, Smad9, Smad1, Ppp2r1b, Neo1, Smad3, Tgfbr2, Smad6, Inhbb, Tfdp1, Smurf1, Nbl1, Fmod, Ep300, Mapk3, Mapk1, Bmpr2) signaling pathways. In certain embodiments, the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97 induced genes that are involved in bone formation, musculoskeletal system development, tissues, and organ morphogenesis.

In certain embodiments, the HKUOT-S2 with a 32 kDa molecular weight isolated from Dioscorea opposita Thunb was characterized using silver staining, MALDI-MS, LC-MS/MS, de novo peptide and N-terminal peptide sequencing techniques.

In certain embodiments, the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97 can be administered to a subject at a dose of about 0.01 mg kg⁻¹ to about 10 mg kg⁻¹ or about 2.18 mg kg⁻¹ safely and effectively.

In certain embodiments, the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97 can enhance bone defect repairs by efficiently modulating cellular functions, such as, for example, macrophage polarizations that regulate pro-inflammatory and anti-inflammatory activities, bone resorption by osteoclasts, and new bone formation by osteoblasts. In certain embodiments, the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97 can enhance osteogenic molecular functions by differentially stimulating osteogenic gene expressions that promote biomineralization and increased BMD to facilitate bone defect repairs.

In certain embodiments, the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97 can induce biological functions and processes, such as, for example, osteoblasts and osteoclast differentiations and musculoskeletal morphogenesis and development to yield the desired new bone formation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1J. HKUOT-S2 significantly enhanced bone defect repairs in vivo. FIGS. 1A-1B) μCT scans revealed that HKUOT-S2 treatments progressively enhanced bone defect healing. FIGS. 1C-1G, FIG. 1J) HKUOT-S2 treatments significantly increased BV/TV, BMD, TMD, Tb.th, Tb. N and BS/TV. FIG. 1H) HKUOT-S2 treatments significantly decreased Tb. Sp. FIG. 1I) HKUOT-S2 treatments have no significant effects on BS/BV. One-way ANOVA followed by Turkey's test post-hoc comparisons for normal distribution and Kruskal-Wallis post-hoc comparisons for non-parametric tests were used for statistical analyses using Prism 5 software. The values were shown as mean±SEM, n=5. X, 2X and 4X=1.09 mgKg⁻¹, 2.18 mgKg⁻¹ and 4.36 mgKg⁻¹ (4X) HKUOT-S2 treatments respectively.*p<0.05, **p<0.01, ***p<0.001 and ****p<0.0001 were considered significant.

FIG. 2A-2L. HKUOT-S2 significantly enhanced bone defect repairs in vivo. FIGS. 2A-2B) fluorochrome labeling showed that HKUOT-S2 treatments enhanced new bone formation. FIGS. 2C-2E) H&E, Giemsa Masson-Goldner trichrome staining showed that HKUOT-S2 treatments enhanced bone mineralization and fractur repairs. FIGS. 2F-2G) 2.18 and 4.36 mg kg⁻¹ HKUOT-S2 significantly decreased TRAP+ cells at bone defect sites. FIGS. 2H-2I) 2.18 and 4.36 mg kg⁻¹ HKUOT-S2 treatments decreased TRAP+ cells but increased ALP+ cells at the growth plates. FIG. 2J) Low magnification (X900) TEM images illustrated normal cell morphology among all the experimental groups. FIG. 2K) High magnification TEM images (X5900) showed that osteoblasts had normal ultrastructure with apparently euchromatic nuclei in the HKUOT-S2 treatment groups. FIG. 2L) High magnification TEM images (X5900) showed that osteoclasts had normal ultrastructure with apparently heterochromatic nuclei in the HKUOT-S2 treatment groups. One-way ANOVA followed by Turkey's test post-hoc comparisons for normal distribution and Kruskal-Wallis post-hoc comparisons for non-parametric tests were used for statistical analyses using Prism 5 software. The values were shown as mean±SEM, X, 2X and 4X=1.09 mgKg⁻¹, 2.18 mgKg⁻¹ and 4.36 mgKg⁻¹ (4X) HKUOT-S2 treatments respectively. *p<0.05, **p<0.01,***p<0.001 and ****p<0.0001 were considered significant. Histological image magnification=×20

FIGS. 3A-3H. HKUOT-S2 significantly increased ALP and OCN levels to enhance bone defect repairs. FIGS. 3A-3D) HKUOT-S2 treatments significantly increased Alp, Bglap1, Bglap1, and Runx2 expressions in the defective femurs. FIGS. 3E-3H) HKUOT-S2 treatments significantly increased BALP and OCN proteins levels in both serum and bone lysate in vivo. One-way ANOVA followed by Turkey's test post-hoc comparisons for normal distribution and Kruskal-Wallis post-hoc comparisons for non-parametric tests were used for statistical analyses using Prism 5 software. The values were shown as mean±SEM, X, 2X and 4X=1.09 mgKg⁻¹, 2.18 mgKg⁻¹ and 4.36 mgKg⁻¹ (4X) HKUOT-S2 treatments respectively. *p<0.05, **p<0.01 were considered significant

FIGS. 4A-4M Transcriptome analysis of HKUOT-S2-induced bone defect repairs. FIG. 4A) Venn diagrams of HKUOT-S2-induced differentially expressed genes (left), differentially upregulated (middle) and downregulated genes (right). FIG. 4B) Pie chart showing the distribution of HKUOT-S2-induced differentially expressed genes. FIG. 4C) Heatmap of HKUOT-S2-induced most common differentially expressed genes. The heatmap was plotted based on z-score of selected genes across samples. FIG. 4D) Both X and 2X HKUOT-S2 significantly enriched GO terms associated with neutrophils differentiations, development, and functions. FIG. 4E) Both X and 2X HKUOT-S2 significantly enriched GO terms associated with monocytes and macrophage differentiations functions. FIG. 4F) All the HKUOT-S2 treatments significantly enriched GO terms associated with macrophage fusion. FIG. 4G) All the HKUOT-S2 treatments significantly enriched GO terms associated with osteoclasts differentiation, development and fusion. FIG. 4H) Both X and 2X HKUOT-S2 treatment significantly enriched GO terms associated stem cells development, differentiations, and functions. FIG. 4I) All the HKUOT-S2 treatments significantly enriched GO terms associated with osteoblasts proliferations, differentiation and functions. FIG. 4J) Significantly enriched KEGG pathways common to all the HKUOT-S2 treatment groups. FIG. 4K) Both X and 2X HKUOT-S2 significantly enriched KEGG pathways. FIG. 4L) Significantly enriched GO terms common to all the HKUOT-S2 treatments. FIG. 4M) Both X and 2X HKUOT-S2-induced significantly enriched KEGG pathways related to the regulation of mTOR complex and signaling pathway. One-way ANOVA followed by Turkey's test post-hoc comparisons for normal distribution and Kruskal-Wallis post-hoc comparisons for non-parametric tests were used for statistical analyses using Prism 5 software. The values were shown as mean±SEM, n=3. X, 2X and 4X=1.09 mgKg⁻¹, 2.18 mgKg⁻¹ and 4.36 mgKg⁻¹ (4X) HKUOT-S2 treatments respectively. *p<0.05, were considered significant

FIGS. 5A-5L. Validation of AMPK, mTOR and BMP, signaling pathway related genes. FIGS. 5A-5H) HKUOT-S2 treatments significantly increased Prkaa1, Prkaa2, Prkab1, Prkab2, Prkag1, Prkag2, Prkag3 and Mtor. FIGS. 5I-5L) HKUOT-S2 treatments significantly increased BMP signaling related genes such as Bmp2, Bmp7 and Bmpr2 but not Tgfβr2. One-way ANOVA followed by Turkey's test post-hoc comparisons for normal distribution and Kruskal-Wallis post-hoc comparisons for non-parametric tests were used for statistical analyses using Prism 5 software. The values were shown as mean±SEM, X, 2X and 4X=1.09 mgKg⁻¹, 2.18 mgKg⁻¹ and 4.36 mgKg⁻¹ (4X) HKUOT-S2 treatments respectively. *p<0.05, **p<0.01 were considered significant

FIGS. 6A-6L. HKUOT-S2 enhanced hMSCs-osteoblast and macrophage polarization and differentiation. FIG. 6A) HKUOT-S2 enhanced osteoblast differentiation. FIGS. 6B-6C) HKUOT-S2 enhanced M1 macrophage polarization. FIGS. 6D-6E) HKUOT-S2 enhanced M2 macrophage polarization. FIG. 6F) HKUOT-S2 increased double-stained CD206 and MGL-1 positive M2 macrophages. HKUOT-S2 increased CD206 positive M1 macrophages. FIG. 6G) HKUOT-S2 increased an M2 macrophage marker, ARG-1 protein level in M1 macrophages. FIG. 6H) HKUOT-S2 increased an anti-inflammatory gene, Ampka1, in M1 macrophages. FIG. 6I) HKUOT-S2 decreased CCL17, CCL22, CXCL16, GDF-15, OPN but increased CD14 and CD54 cytokines in M1 macrophage CM. HKUOT-S2 also increased G-CSF but decreased GDF-15 cytokines in the M2 macrophage CM. FIGS. 6J-6K) HKUOT-S2-treated M1 macrophage CM significantly increased osteoblast biomineralization. FIGS. 6J, 6L) HKUOT-S2-treated M2 macrophage CM significantly increased osteoblast biomineralization. One-way ANOVA followed by Turkey's test post-hoc comparisons for normal distribution and Kruskal-Wallis post-hoc comparisons for non-parametric tests were used for statistical analyses using Prism 5 software. The values were shown as mean±SEM, n=5. *p<0.05, **p<0.01,***p<0.001 and ****p<0.0001 were considered significant. Diff med=differentiation medium, CM=conditioned media.

FIG. 7A-7O. HKUOT-S2 modulates mTOR1/4E-BP1/AKT1 axis to promote osteogenesis. FIG. 7A) XL388-induced mTOR inhibition has no effects on ALP expression. FIGS. 7B-7F) HKUOT-S2 blocks the XL388-induced mTOR inhibition to increase RUNX2, mTOR1, 4E-BP1, AKT1 and S6K1 expressions to enhance osteogenesis. FIGS. 7G-7M) HKUOT-S2 blocks the XL388-induced mTOR inhibitory effects to increase the phosphorylation of mTOR1 and 4E-BP1 proteins. FIG. 7N) HKUOT-S2 treatment also increased total AKT1 protein level. FIG. 7O) Proposed mechanism by which HKUOT-S2 modulate mTOR1/4E-BP1/AKT1 axis to promote osteogenesis. The values are shown as mean±SEM, X-125=125 nM XL388, (n=4). *p<0.05 were considered significant under One-Way ANOVA.

FIGS. 8A-8H. HKUOT-S2 Enhances Neuron Differentiation and Maturation in Vivo. FIG. 8A, FIG. 8C, FIG. 8E, FIG. 8F, FIG. 8H) HKUOT-S2 treatment significantly increased neuron differentiation and maturation genes, Ngn1, TH, Gap 43, Map2 and Eno2 expressions. FIG. 8B, FIG. 8D, FIG. 8G) HKUOT-S2 treatment had no significant effects on neuron differentiation and maturation genes Ngn2, Neurod1 and Tuj1 expressions.

FIGS. 9A-9F. HKUOT-S2 significantly enriched pathways and biological processes associated growth, development and maturation of the neurons, nerves, and brain. FIG. 9A) HKUOT-S2 treatments significantly enriched GO terms associated with axon, cell body and dendrite development. FIG. 9B) Transcriptomic data showed that HKUOT-S2 treatment significantly enriched GO terms associated with axonal development, regeneration, and injury repairs in bone defect model. FIGS. 9C-9D) HKUOT-S2 treatment significantly enriched GO terms associated neuronal growth, development, differentiation and functions. FIG. 9E) HKUOT-S2 treatment significantly enriched GO terms associated nerve morphogenesis and structural organization. FIG. 9F) HKUOT-S2 treatment significantly enriched GO terms associated with brain development.

One-way ANOVA followed by Turkey's test post-hoc comparisons for normal distribution and Kruskal-Wallis post-hoc comparisons for non-parametric tests were used for all statistical analyses using Prism 5 software. The values were shown as mean±SEM. *p<0.05, **p<0.01, and ***p<0.001 were considered significant.

FIGS. 10A-10H. HKUOT-S2 enhanced neuropeptide genes expression and functions. FIGS. 10A-10D) qPCR results showed that HKUOT-S2 treatments significantly increased Cox2, Ptges, Ep4 and Calca expressions in bone defect model. FIG. 10E) qPCR results showed that HKUOT-S2 had no significant effects on calcitonin receptor-like receptor gene, Crlr expressions. FIGS. 10F-10G) Transcriptomic data showed that HKUOT-S2 treatment significantly enriched GO terms associated with some neuropeptide genes in bone defect model. FIG. 10H) Transcriptomic data showed that HKUOT-S2 treatments significantly enriched pathways and biological processes associated with neuropeptide synthesis, release and activities in bone defect model.

FIGS. 11A-11E: HKUOT-S2 treatment promotes neuron differentiation. FIGS. 11A, 111B) HKUOT-S2 treatment has no effects on Neuro2A cell viability. FIGS. 11C-11E) HKUOT-S2 treatment promotes Neuro2A-neuron cell differentiation.

FIG. 12A-12H. HKUOT-S2 prevented OVX-induced osteoporosis development in vivo. FIG. 12A) Transcriptomic data showed that HKUOT-S2 treatment significantly enriched GO terms associated with the regulation of estrogen receptor binding and signaling pathway. FIG. 12B) HKUOT-S2 treatment prevented bone loss in OVX mice. FIG. 12C) HKUOT-S2 treatment increased bone volume in OVX mice. FIG. 12D) HKUOT-S2 treatment decreased bone surface to volume ratio in OVX mice. FIG. 12E) HKUOT-S2 treatment increased bone surface density in OVX mice. FIG. 12F) HKUOT-S2 treatment increased trabecular thickness in OVX mice. FIG. 12G) HKUOT-S2 treatment increased trabecular number in OVX mice. FIG. 12H) HKUOT-S2 treatment decreased trabecular separation in OVX mice. One-way ANOVA followed by Turkey's test post-hoc comparisons for normal distribution and Kruskal-Wallis post-hoc comparisons for non-parametric tests were used for all statistical analyses using Prism 5 software. The values were shown as mean±SEM, n=8, *p<0.05, **p<0.01, ***p<0.001 and ****p<0.0001 were considered significant

FIGS. 13A-13H. HKUOT-S2 suppressed progressive dexamethasone (Dex)-induced osteoporosis development. FIG. 13A) Both 1.09 and 2.18 mg kg⁻¹ HKUOT-S2 significant enriched biological processes and functions associated with the response to glucocorticoid stimuli including dexamethasone (Dex) the in wild type mice. FIG. 13B) HKUOT-S2 inhibited bone loss in Dex treated mice at week 4 of experimental endpoint. FIG. 13C) HKUOT-S2 treatment increased bone volume in Dex treated mice. FIG. 13D) HKUOT-S2 treatment decreased bone surface to volume ratio in Dex treated mice. FIG. 13E) HKUOT-S2 treatment increased bone surface density in Dex treated mice. FIG. 13F) HKUOT-S2 treatment increased trabecular thickness in Dex treated mice. FIG. 13G) HKUOT-S2 treatment increased trabecular number in Dex treated mice. FIG. 13H) HKUOT-S2 treatment decreased trabecular separation in Dex treated mice.

FIGS. 14A-14B. HKUOT-S2 treatments enhanced glucose metabolism and insulin functions in wild type mice. FIGS. 14A-14B) Both 1.09 and 2.18 mg kg⁻¹ HKUOT-S2 treatments significantly enriched several signaling pathways, biological processes and functions associated with glucose metabolism and insulin functions in wild type male mice.

FIGS. 15A-15H. HKUOT-S2 treatments increased functional pancreatic 3-cells gene expressions in vitro. FIG. 15A) HKUOT-S2 treatment had no significant effects on INS-1E cell viability. FIG. 15B-15H) HKUOT-S2 treatments apparently increased functional pancreatic 3-cells genes expressions in INS-1E cells.

FIGS. 16A-16B. Effects of HKUOT-S2 treatment on reproduction and embryonic development in vivo. FIG. 16A) Both 1.09 and 2.18 mg kg⁻¹ HKUOT-S2 treatments significantly enriched biological processes and functions associated with oogenesis, spermatogenesis, and reproductive processes in the wild type mice. FIG. 16B) Both 1.09 and 2.18 mg kg⁻¹ HKUOT-S2 treatments significantly enriched biological processes and functions associated with embryonic development in the wild type mice.

FIGS. 17A-17I. HKUOT-S2 isolation and characterization. FIG. 17A) Crude Dioscorea opposita Thunb protein extract. HKUOT-D3 protein isolated from the crude Dioscorea opposita Thunb protein extract. HKUOT-P1 protein isolated from the protein fraction HKUOT-D3. FIG. 17B) HKUOT-S2 protein purified from HKUOT-P1. FIG. 17C) Silver staining of HKUOT-S2 and HKUOT-P1 proteins in 15% SDS-PAGE. FIG. 17D) HKUOT-S2 molecular weight determination by mass spectrometry. FIGS. 17E-17H) Analysis of HKUOT-S2 de novo peptide sequencing on PEAKS Studio X-Pro. FIG. 17I) The molecular weight (MW) of HKUOT-S2 was predicted mathematically using the standard curve of the calibrated Superdex 75 Increase 10/300 GL column information and Kav equation.

FIGS. 18A-18W. HKUOT-S2 had no toxic effects on mice. FIG. 18A) HKUOT-S2 did not induce hemolysis of mouse blood. FIGS. 18B, 18C) HKUOT-S2 inhibits hypotonic (0.45% saline)-induced hemolysis. FIG. 18D) Assessment of the acute toxic effects of HKUOT-S2 body weights, liver and kidney histology and gene expressions, hematocrit, and clinical biochemistry. FIG. 18E). HKUOT-S2 had no effects on body weight. FIGS. 18F, 18G) HKUOT-S2 had no effects on the liver and kidney histology. FIGS. 18H-18K) HKUOT-S2 had no effects on the liver gene expressions. FIGS. 18L-18O) HKUOT-S2 had no effects on the kidney gene expressions. FIGS. 18P-18Q) HKUOT-S2 treatments had no effects on haematocrit. FIGS. 18R-18W) Clinical biochemistry of the sera showed that HKUOT-S2 treatment had no effects on serum levels of ALP, ALT, AST, ALP/AST, creatinine, and urea in vivo. One-way ANOVA was used as a statistical tool for analysis. The values are shown as mean±SEM (n=9 per group). *p<0.05 were considered significant. HTC=Haematocrit.

FIGS. 19A-19F. Transcriptome analysis of HKUOT-S2-induced bone fracture repairs. FIG. 19A, 19B) 1.09 mg kg⁻¹ HKUOT-S2 significantly enriched KEGG pathways. FIG. 19C, 19D) 2.18 mg kg⁻¹ HKUOT-S2 significantly enriched KEGG pathways. FIG. 19E) Significantly enriched GO terms common to both 1.09 and 2.18 mg kg⁻¹ HKUOT-S2 treatments. FIG. 19F) 4.36 mg kg⁻¹ HKUOT-S2 significantly enriched GO terms. One-way ANOVA was used as a statistical tool for analysis. The values are shown as mean±SEM (n=3). *p<0.05 were considered significant.

FIGS. 20A-20N. Yam protein extracts increased RUNX2 expression in differentiated osteoblasts. FIGS. 20A-20D) Crude yam protein extract, HKUOT-D3, HKUOT-P1 and HKUOT-S2 have no effects on hTMSCs and RAW264.7 cell proliferation and viability. FIGS. 20E-20L) Crude yam protein extract increased RUNX2 expression hMSCs and MC3T3-1E cells-derived osteoblasts. FIGS. 20M, 20N) 0.01μ gml⁻¹ HKUOT-S2 de novo peptide sequence TKSSLPGQTK (SEQ ID NO: 83) promoted osteoblast differentiation by increasing ALP and COL-1A expressions in vitro.

FIGS. 21A-21B. HKUOT-S2 modulates mTOR1/4E-BP1 axis to promote osteogenesis. FIG. 21A) HKUOTS-2 promotes hMSCs-osteoblast differentiation by increasing ALP expression. FIG. 21B) HKUOT-S2 blocks the XL388-induced mTOR inhibitory effects to increase the phosphorylation of mTOR1 and 4E-BP1 proteins and increase total AKT1 protein levels. The values are shown as mean±SEM, (n=4). *p<0.05 were considered significant under One-Way ANOVA.

FIGS. 22A-22H. HKUOT-S2 and SEQ ID NO: 83 (TK) significantly suppressed osteoporosis development. FIG. 22A) μCT scans of the femurs of the sham control, OVX control, HKUOT-S2 and TK treatment groups. FIGS. 22B-22H) μCT analysis results of BV/TV, BS/TV ratio, Tb.th, Tb. N, BS/BV and Tb. Sp of the femur. The values were shown as mean±SEM, n=3. HKUOT-S2=2.18 mg/kg, TK=0.5 mg/kg treatments respectively. *p<0.05, **p<0.01, ***p<0.001 and ****p<0.0001.

FIGS. 23A-23F. HKUOT-S2 and SEQ ID NO: 83 (TK) significantly promote osteoblast differentiation. FIG. 23A) HKUOT-S2 and SEQ ID NO: 83 treatments increased ERα expression. FIG. 23B) HKUOT-S2 and SEQ ID NO: 83 treatments increased ALP expression. FIGS. 23C-23D) HKUOT-S2 and SEQ ID NO: 83 treatments increased osteoblast ALP activities. FIGS. 23E-23F) HKUOT-S2 and SEQ ID NO: 83 treatments increased osteoblast biomineralization. The values were shown as mean±SEM, n=4. *p<0.05, **p<0.01, ***p<0.001 and ****p<0.0001.

FIGS. 24A-24J. HKUOT-S2 promotes osteoblast differentiation by modulating estrogen receptors. FIGS. 24A-24C) HKUOT-S2 treatment increased ERα and GPR30 expressions but had no effects on ERβ expression. FIGS. 24D-24F) HKUOT-S2 treatment increased ALP, COL1A1 and RUNX2 expressions during osteoblast differentiation. FIGS. 24G-24H) HKUOT-S2 treatment increased osteoblast ALP activities. FIGS. 24I-24J) HKUOT-S2 treatment increased osteoblast biomineralization. The values were shown as mean±SEM, n=4. *p<0.05, **p<0.01, ***p<0.001 and ****p<0.0001.

FIGS. 25A-25D. HKUOT-S2 treatment upregulates estrogen receptors in OVX mice. FIGS. 25A-25D) HKUOT-S2 treatment increased ERα, ERβ and GPR30 protein expressions in bone tissues. The values were shown as mean±SEM, n=4. *p<0.05, **p<0.01, ***p<0.001 and ****p<0.0001.

FIGS. 26A-26G. HKUOT-S2 significantly suppressed osteoporosis development in lumbar vertebrae. FIG. 26A) μCT scans of the L5 of the sham control, OVX control, HKUOT-S2 treatment groups. FIGS. 26B-26G) μCT analysis of BV/TV, BS/TV ratio, Tb.th, Tb. N, BS/BV and Tb. Sp of the L5. The values were shown as mean±SEM, n=8. HKUOT-S2=2.18 mg/kg, TK=0.5 mg/kg treatments respectively. *p<0.05, **p<0.01, ***p<0.001 and ****p<0.0001.

FIG. 27. Determination of Molecular weight (MW) of S2 and S3 proteins using Kav equation. The standard curve of column was calibrated with proteins of known MV.

### BRIEF DESCRIPTION OF THE SEQUENCES

SEQ ID NO: 1: Alp primer forward
SEQ ID NO: 2: Alp primer reverse
SEQ ID NO: 3: Col primer forward
SEQ ID NO: 4: Col primer reverse
SEQ ID NO: 5: Opn primer forward
SEQ ID NO: 6: Opn primer reverse
SEQ ID NO: 7: Runx 2 primer forward
SEQ ID NO: 8: Runx 2 primer reverse
SEQ ID NO: 9: Arg-1 primer forward
SEQ ID NO: 10: Arg-1 primer reverse
SEQ ID NO: 11: Mgl-1 primer forward
SEQ ID NO: 12: Mgl-1 primer reverse
SEQ ID NO: 13: Cd206 primer forward
SEQ ID NO: 14: Cd206 primer reverse
SEQ ID NO: 15: Ym1 primer forward
SEQ ID NO: 16: Ym1 primer reverse
SEQ ID NO: 17: Socs3 primer forward
SEQ ID NO: 18: Socs3 primer reverse
SEQ ID NO: 19: Tnfa primer forward
SEQ ID NO: 20: Tnfa primer reverse
SEQ ID NO: 21: iNOS primer forward
SEQ ID NO: 22: iNOS primer reverse
SEQ ID NO: 23: IL-6 primer forward
SEQ ID NO: 24: IL-6 primer reverse
SEQ ID NO: 25: IL-1β primer forward
SEQ ID NO: 26: IL-1β primer reverse
SEQ ID NO: 27: Mcp-1 primer forward
SEQ ID NO: 28: Mcp-1 primer reverse
SEQ ID NO: 29: Prkaa1 primer forward
SEQ ID NO: 30: Prkaa1 primer reverse
SEQ ID NO: 31: Prkaa2 primer forward
SEQ ID NO: 32: Prkaa2 primer reverse
SEQ ID NO: 33: Prkab1 primer forward
SEQ ID NO: 34: Prkab1 primer reverse
SEQ ID NO: 35: Prkab2 primer forward
SEQ ID NO: 36: Prkab2 primer reverse
SEQ ID NO: 37: Prkag1 primer forward
SEQ ID NO: 38: Prkag1 primer reverse
SEQ ID NO: 39: Prkag2 primer forward
SEQ ID NO: 40: Prkag2 primer reverse
SEQ ID NO: 41: Prkaig3 primer forward
SEQ ID NO: 42: Prkag3 primer reverse
SEQ ID NO: 43: Gapdh primer forward
SEQ ID NO: 44: Gadph primer reverse
SEQ ID NO: 45: Bglap1 primer forward
SEQ ID NO: 46: Bglap1 primer reverse
SEQ ID NO: 47: Bglap2 primer forward
SEQ ID NO: 48: Bglap2 primer reverse
SEQ ID NO: 49: Got1(Ast) primer forward
SEQ ID NO: 50: Got1(Ast) primer reverse
SEQ ID NO: 51: Gpt1(Ast) primer forward
SEQ ID NO: 52: Gpt1(Ast) primer reverse
SEQ ID NO: 53: Ckb (Creatine) primer forward
SEQ ID NO: 54: Ckb (Creatine) primer reverse
SEQ ID NO: 55: mTOR primer forward
SEQ ID NO: 56: mTOR primer reverse
SEQ ID NO: 57: Bmp2 primer forward
SEQ ID NO: 58: Bmp2 primer reverse
SEQ ID NO: 59: Bmrp2 primer forward
SEQ ID NO: 60: Bmpr2 primer reverse
SEQ ID NO: 61: Bmp7 primer forward
SEQ ID NO: 62: Bmp7 primer reverse
SEQ ID NO: 63: Tgfbr2 primer forward
SEQ ID NO: 64: Tgfbr2 primer reverse
SEQ ID NO: 65: ALP primer forward
SEQ ID NO: 66: ALP primer reverse
SEQ ID NO: 67: COL1A primer forward
SEQ ID NO: 68 COL1A primer reverse
SEQ ID NO: 69: OPN primer forward
SEQ ID NO: 70: OPN primer reverse
SEQ ID NO: 71: RUNX2 primer forward
SEQ ID NO: 72: RUNX2 primer reverse
SEQ ID NO: 73: GAPDH primer forward
SEQ ID NO: 74: GAPDH primer reverse
SEQ ID NO: 75: 4EBP1 primer forward
SEQ ID NO: 76: 4EBP1 primer reverse
SEQ ID NO: 77: AKT1 primer forward
SEQ ID NO: 78: AKT1 primer reverse
SEQ ID NO: 79: S6K1 primer forward
SEQ ID NO: 80: S6K1 primer reverse
SEQ ID NO: 81: HKUOT-S2 peptide
SEQ ID NO: 82: HKUOT-S2 peptide
SEQ ID NO: 83: HKUOT-S2 peptide
SEQ ID NO: 84: HKUOT-S2 peptide
SEQ ID NO: 85: HKUOT-S2 peptide
SEQ ID NO: 86: HKUOT-S2 peptide
SEQ ID NO: 87: HKUOT-S2 peptide
SEQ ID NO: 88: HKUOT-S2 peptide
SEQ ID NO: 89: HKUOT-S2 peptide
SEQ ID NO: 90: HKUOT-S2 peptide
SEQ ID NO: 91: HKUOT-S2 peptide
SEQ ID NO: 92: HKUOT-S2 peptide
SEQ ID NO: 93: HKUOT-S2 peptide
SEQ ID NO: 94: HKUOT-S2 peptide
SEQ ID NO: 95: HKUOT-S2 peptide
SEQ ID NO: 96: HKUOT-S2 peptide
SEQ ID NO: 97: HKUOT-S2 peptide

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994). The term nucleic acid is used interchangeably with gene, cDNA, and mRNA encoded by a gene.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, the term “sample” refers to a sample comprising at least one protein, peptide, or nucleic acid, including a blood sample. In one embodiment, a “biological sample,” as that term is used herein, refers to a sample obtained from a subject, wherein the sample comprises at least one protein, peptide, or nucleic acid. While not necessary or required, the term “biological sample” is intended to encompass samples that are processed prior to assaying using the systems and methods described herein.

As used herein, the term “subject” refers to a plant or animal, particularly a human, from which a biological sample is obtained or derived from. The term “subject” as used herein encompasses both human and non-human animals. The term “non-human animals” includes all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. In some embodiments, the term “subject” refers to a mammal, including, but not limited to, murines, simians, humans, felines, canines, equines, bovines, mammalian farm animals, mammalian sport animals, and mammalian pets. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a protein, peptide, or composition thereof that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

In some embodiments of the invention, the method comprises administration of multiple doses of the compositions of the subject invention. The method may comprise administration of therapeutically effective doses of a composition comprising the protein, peptide, or composition thereof of the subject invention as described herein three times a week, once a week, or more frequency. In some embodiments, doses are administered over the course of 1 week, 2 weeks, or more than 3 weeks. Moreover, treatment of a subject with a therapeutically effective amount of the compositions of the invention can include a single treatment or can include a series of treatments. It will also be appreciated that the effective dosage of a protein, peptide, or composition thereof used for treatment may increase or decrease over the course of a particular treatment. Changes in dosage may result and become apparent from the results of diagnostic assays or imaging techniques for detecting or visualizing bone known in the art. In some embodiments of the invention, the method comprises administration of the compositions at several time per day, including but not limiting to 2 times per day, 3 times per day, and 4 times per day.

As used in herein, the terms “identical” or “percent identity”, in the context of describing two or more polynucleotide sequences, refer to two or more sequences or subsequences that are the same or have a specified percentage of nucleotides that are the same over the compared region. For example, a homologous nucleotide sequence used in the method of this invention has at least 80% sequence identity, preferably 85%, 90%, 91%, 92%, 93, 94%, 95%, 96%, 97%, 98%, 99%, or 100% identity, to a reference sequence, when compared and aligned for maximum correspondence over a comparison window, or over a designated region as measured using a comparison algorithms or by manual alignment and visual inspection. With regard to polynucleotide sequences, this definition also refers to the complement of a test sequence.

As used in herein, the terms “identical” or “percent identity”, in the context of describing two or more amino acid sequences, refer to two or more sequences or subsequences that are the same or have a specified percentage of amino acids that are the same over the compared region. For example, a homologous nucleotide sequence used in the method of this invention has at least 80% sequence identity, preferably 85%, 90%, 91%, 92%, 93, 94%, 95%, 96%, 97%, 98%, 99%, or 100% identity, to a reference sequence, when compared and aligned for maximum correspondence over a comparison window, or over a designated region as measured using a comparison algorithm or by manual alignment and visual inspection. With regard to amino acid sequences, this definition also refers to the complement of a test sequence.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Protein HKUOT-S2 and Compositions Thereof

In certain embodiments, the compositions of the subject invention comprise a protein isolated from Dioscorea opposita Thunb: HKUOT-S2. The HKUOT-S2 protein can be isolated from the Dioscorea opposita Thunb by successive ion exchange, hydrophobic interaction, and high-resolution size-exclusion chromatographic techniques. The molecular weight of HKUOT-S2 protein is 32 kDa as determined by Mathematical model, silver staining and mass spectrometry. HKUOT-S2 is further characterized using high resolution liquid chromatography tandem mass spectrometry (LC-MS/MS), de novo peptide and N-terminal peptide sequencings. In certain embodiments, the compositions comprise a peptide selected from the HKUOT-S2 protein, at least one peptide according to at least one of SEQ ID NO: 81-97 or sequence having at least 90% sequence identity to SEQ ID NO: 81-97, or a protein comprising an amino acid sequence according to at least one of SEQ ID NO: 81-97 or sequence having at least 90% sequence identity to SEQ ID NO: 81-97.

In certain embodiments, the HKUOT-S2 protein, at least one peptide according to at least one of SEQ ID NO: 81-97 or sequence having at least 90% sequence identity to SEQ ID NO: 81-97, or a protein comprising an amino acid sequence according to at least one of SEQ ID NO: 81-97 or sequence having at least 90% sequence identity to SEQ ID NO: 81-97 may be added to compositions at concentrations of about 0.0001 to about 50% by weight (wt %), preferably about 0.01 to about 10 wt %, and most preferably about 0.1% to about 10 wt %. In another embodiment, the HKUOT-S2 protein, at least one peptide according to at least one of SEQ ID NO: 81-97 or sequence having at least 90% sequence identity to SEQ ID NO: 81-97, or a protein comprising an amino acid sequence according to at least one of SEQ ID NO: 81-97 or sequence having at least 90% sequence identity to SEQ ID NO: 81-97 can be in combination with an acceptable carrier and/or excipient, in that the protein or peptide may be presented at concentrations of about 0.0001 to about 50% (v/v), preferably, about 0.01 to about 10% (v/v), more preferably, about 0.1 to about 10% (v/v).

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, intracerebroventricularly or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In one embodiment, the compositions of the subject invention can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

### Methods of Using HKUOT-S2 and Composition Thereof

In certain embodiments, the HKUOT-S2 protein, at least one peptide according to at least one of SEQ ID NO: 81-97 or sequence having at least 90% sequence identity to SEQ ID NO: 81-97, or a protein comprising an amino acid sequence according to at least one of SEQ ID NO: 81-97 or sequence having at least 90% sequence identity to SEQ ID NO: 81-97 can be administered to a subject. Any means of administration that can permit a peptide or protein to contact cells in a subject, including, for example, orally, intravenously, intraperitoneally, intramuscularly, intrathecally, or subcutaneously, are envisioned in the subject methods. In certain embodiments, a peptide or protein can contact cells of subject, including, for example, macrophages, osteoblasts, chondrocytes, osteoclasts, and MSCs.

In certain embodiments, the proteins and/or peptides of the subject invention can induce macrophage polarization and human MSCs (hMSCs)-derived osteoblasts mineralization. In certain embodiments, the proteins and/or peptide of the subject invention can modulate genes enriched in focal adhesion, mTOR, AMPK and BMP signaling pathways. The proteins and/or peptide of the subject invention can also induce genes that are involved in bone cell development (Ptpn6, Fli1, Med1, Tnfsf11, Rabgap1l, Pip4k2a, Src, Sh2b3, Zfpm1, Ep300, Nbeal2) and new bone formation. (Tgfb1, Rsad2, Hif1a, Ltbp3, Alox5, Fam20c, Smad7, Ddr2, Isg15, Smad6, Suv39h1, S1pr1, Adrb2, Sgms2, Mapk14, Bcl2, Kl, Mapk3, Mapk1, Sox11, Zbtb16, and Adgrv1). In certain embodiments, the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97 can promote human mesenchymal stem cells (hMSCs) to osteoblast differentiation by increasing oestrogen receptor α (ERα), oestrogen receptor β (ERβ), and ALP mRNA expression. In certain embodiments, the HKUOT-S2 protein, at least one peptide according to at least one of SEQ ID NO: 81-97 or sequence having at least 90% sequence identity to SEQ ID NO: 81-97, or proteins comprising an amino acid sequence according to at least one of SEQ ID NO: 81-97 or sequence having at least 90% sequence identity to SEQ ID NO: 81-97 can upregulate osteogenic gene expressions of ALP, COL1A1 and RUNX2.

In certain embodiments, the proteins and/or peptides of the subject invention can enhance bone defect repairs by efficiently modulating cellular functions, such as, for example, macrophage polarizations that regulate pro-inflammatory and anti-inflammatory activities, bone resorption by osteoclasts, and new bone formation by osteoblasts. The proteins and/or peptides of the subject invention can also enhance osteogenic molecular functions by differentially stimulating osteogenic gene expressions that promoted biomineralization and increased BMD to facilitate bone defect repairs. In certain embodiments, the proteins and/or peptides of the subject invention induce multiple osteogenic signaling pathways, such as, for example, focal adhesion, AMPK, PI3K/Akt/mTOR and BMP/TGF-0 signaling pathways as well as biological functions and processes, such as, for example, osteoblasts and osteoclast differentiations, musculoskeletal morphogenesis, and development to yield the desired new bone formation.

In certain embodiments, the proteins and/or peptides of the subject invention are non-hemolytic and protect the red blood cells (RBCs) against hypotonic-induced hemolysis (RBCs rupture). In certain embodiments, the proteins and/or peptides of the subject invention induce differentially expressed genes that modulate chondrocytes (Slc39a14, Col11a2, Ift80, Adamts12), osteoblasts (Fbxo5, Gli3, Men1, Il6, Fam20c, Ddr2, Wwtr1, Ctnnbip1, Ltf, Cebpb, Sox11, Jund), and osteoclast (Tcirg1, Tgfb1, Gab2, Epha2, Tnfsf11, Tnf, Csf1r, Tnfrsf11a, Traf6, Tyrobp, Trf, Mitf, Snx10, Junb, Mapk14, Fcer1g, Fos, Tnfsf11, Creb1, Car2, Klf10) differentiations, endochondral (Mmp14, Col1a1, Ift80, Mmp16, Mmp13, Col13a1) and intramembranous (Col1a1, Mmp2) ossifications, cartilage development (Col1a1, Ift80, Mmp13, Bmp1, Col11a2, Cfh, Col11a1, Bmp3, Bgn, Smad1, Mmp13), odontogenesis (Lamb1, Id3, Fst, Lrp4, Slc4a2, Pdgfra, Fgfr2, Phex), neurogenesis (: Fabp7, Btbd1, Lrp4, Tal1, Cit, Dbn1, Cdk5rap2, Nup133, Serpinf1, Mdk, Ngf, Ptprd, Mtor, Plxna4, Plxnd1, Aspm, Lif Eef2k, Zfp335, Shtn1, Robo2, Kdr), neuron (Cfh, Ngf Gba2, Fgfr2, Lif, Hs6st1, Robo2, Htra2, Nanos1) and axon (Mtr, Mmp2, Camsap2) development, neuropeptides synthesis (Sema4f Sema6a, Ptges2, Map2, Sema6c, Sema4b, Ptges3, Sema3c, Sema4a) and enhance liver (Foxm1, Vwf, Med1, Sec63, Xbp1, Stat5b, Ptcd2, Il6, Notch1, Sp3, Aurka, Notch2, Aacs, Ezh2, Atg7, Hmbs, Pkd2, Hlx, Icmt, Onecut1, Zmpste24, Taf10, Arf6, Onecut2, Jun, Klf1, Cebpb, Mpst, Atf7), kidney (Bax, Bloc1s6, Qrich1, Tgfbr1, Ap1b1, Slc5a1, Odc1, Glis2, Sgpl1, Id2, Gli3, Ptcd2, Robo1, Adamts1, Bag6, Pdgfrb, Cat, Bcl2l11, Pds5a, Cux1, Nek1, Ctnnd1, Wfs1, Greb1l, Fmn1, Pou3f3, Pygo2, Agtr1a, Zbtb14, Foxc1, Bcl2, Zbtb16, Mpst, Smo, Glis2, Ednrb, Prom1, Pou3f3, Cd24a, Ext1) and cardiac cell (Tbx2, Id2, Zfpm2, Csrp3, Sin3b, Tbx18, Gjc1, Sgcg, Tbx20, Ccnb1, Mapk14, Mapk1, Tgfbr1, Pi16, G6pdx, Tgfbr2, Parp2, Rgs4, Gsk3a, G6pd2) development.

In certain embodiments, the proteins and/or peptides of the subject invention can increase neuronal function modulating genes such as, for example, Ep4, Ptges, Cox2, Eno2 and Calca to enhance bone fracture repairs. In certain embodiments, the proteins and/or peptides of the subject invention could mechanistically activate the mTOR/4E-BP1 axis to promote osteogenesis.

In certain embodiments, the HKUOT-S2 protein, at least one peptide according to at least one of SEQ ID NO: 81-97 or sequence having at least 90% sequence identity to SEQ ID NO: 81-97, or a protein comprising an amino acid sequence according to at least one of SEQ ID NO: 81-97 or sequence having at least 90% sequence identity to SEQ ID NO: 81-97 may be administered to a subject at a dose of about 0.01 mg kg⁻¹ to about 50 mg kg⁻¹, about 0.1 mg kg⁻¹ to about 10 mg kg⁻¹, about 1.09 mg kg⁻¹, about 2.18 mg kg⁻¹, or about 4.36 mg kg⁻¹.

In certain embodiments, the subject compositions can be used in methods of treating osteoporosis. In certain embodiments, the proteins and/or peptides of the subject invention can inhibit bone loss by suppressing both OVX- and drug (glucocorticoid)-induced osteoporosis development.

In certain embodiments, the subject compositions can be used in methods of osteogenesis and treating new bone formation problems. In certain embodiments, the proteins and/or peptides of the subject invention significantly enhance osteoblast differentiation and functions to promote osteogenesis and new bone formation. The subject compositions can activate the mTOR/4E-BP1/AKT1 axis to promote osteogenesis. The uCT scan can be used to measure the progressive enhanced bone fracture repairs in vivo. The enhanced bone fracture repairs can be evaluated by measuring the percentage, bone volume, bone mineral density, trabecular thickness, separation and number, and/or bone surface area to tissue volume (BS/TV) ratio at the bone defect sites. In certain embodiments, the methods can provide the combination of a quicker repair or a more structurally sound repair.

In certain embodiments, the subject compositions can be used in methods of treating delayed bone fracture repairs. In certain embodiments, the proteins and/or peptides of the subject invention significantly enhance bone fracture repairs and can be used as intervention for delayed bone fracture repairs. Delayed bone healing indicates that the bone fracture takes longer period to heal than normal. In certain embodiments, bone fracture healing can be considered delayed when it takes at least about 3 months to heal. The time frame for delayed healing may vary according to different bone types.

In certain embodiments, the subject compositions can be used in methods of treating inflammatory diseases, such as, for example, rheumatoid arthritis. In certain embodiments, the proteins and/or peptides of the subject invention can modulate M1 and M2 macrophage polarization and maintain the homeostatic balance between M1 and M2 macrophage functions to suppress the development of inflammatory diseases, such as, for example osteoarthritis.

In certain embodiments, the subject compositions can be used in methods of treating spinal cord injuries (SCI). In certain embodiments, the proteins and/or peptides of the subject invention promote neurogenesis and modulate macrophage polarization, which are essential ingredients for SCI repairs.

In certain embodiments, the subject compositions can be used in methods of treating liver and kidney diseases. In certain embodiments, the proteins and/or peptides of the subject invention augment liver and kidney function Ast and Ckb gene expressions. In certain embodiments, the proteins and/or peptides of the subject invention induce differentially expressed genes that are significantly enriched in biological functions and processes that modulate liver and kidney and cardiac development. In certain embodiments, the proteins and/or peptides of the subject invention can inhibit liver related diseases, such as, for example, hepatitis and hepatic cirrhosis and nephropathies, such as, for example, nephritis and nephrosis, through augmentation of key hepatic (Rps6ka1, Il6, Cebpb, Notch2) and renal (Bax, Bloc1s6, Qrich1, Tgfbr1, Ap1b1, SlcSal, Odc1, Glis2, Sgpl1, Id2, Gli3, Ptcd2, Robo1, Adamts1, Bag6, Pdgfrb, Cat, Bcl2l11, Pds5a, Cux1, Nek1, Ctnnd1, Wfs1, Greb1l, Fmn1, Pou3f3, Pygo2, Agtr1a, Zbtb14, Foxc1, Bcl2, Zbtb16, Mpst, Smo, Glis2, Ednrb, Prom1, Pou3f3, Cd24a, Ext1) gene expression, hepatogenesis (Foxm1, Vwf, Med1, Sec63, Xbp1, Stat5b, Ptcd2, Il6, Notch1, Sp3, Aurka, Notch2, Aacs, Ezh2, Atg7, Hmbs, Pkd2, Hlx, Icmt, Onecut1, Zmpste24, Taf10, Arf6, Onecut2, Jun, Klf1, Cebpb, Mpst, Atf7), renal regeneration (Tgfb1, Aqp1, Dlg1, Smad7, Cat, Smad9, Kank2, Smad6, Greb1l, Pou3f3, Foxc1, Bcl2, Smo, Glis2, Gli3, Ednrb, Prom1, Pou3f3, Cd24a, Ext1), and macrophage polarization (Socs3, Tnfα, Arg-1, Mgl-1, Ampka1, Pparg, Lgals9, Havcr2, Gpr137b, Snca, Il1rl1, Cd1d1, Stap1, Atm, Lrrk2, Tlr4, Sphk1, Muc5b, Jund, Ulbp1).

In certain embodiments, the subject compositions can be used in methods of treating cardiovascular related problems. In certain embodiments, the proteins and/or peptides of the subject invention induce differentially expressed genes that are significantly enriched in biological functions and processes which modulate hematopoiesis (Ap2a2, Nfe2l2, Eif2ak2, Zbtb1, Sos2, Ankle1, Foxc1, Kitl, Cxcl1, Atxn1l), angiogenesis (Itgb2, Hk2, Aqp1, Notch4, Nfe2l2, Ptgis, Nr2e1, Xbp1, Hif1a, Sema5a, Cyp1b1, C3, Itgb8, Cxcr2, Jak1, Add1, Ctsh, Tgfbr2, Ccr3, Btg1, Hspb6, Lrg1, Serpine1, Pik3cd, Agtr1a, C5ar1, Sphk1, Chil1), vasculogenesis (Ccm2, Foxm1, Smo, Tgfb1, Epha2, Zmiz1, Sgpl1, Myocd, Zfp36l1, Zfpm2, Itgb8, Spred1, Tbx20, Tgfbr2, Gjc1, Junb), cardiogenesis (Tbx2, Tgfbr1, Zfpm2, Pi16, G6pdx, Tbx20, Tgfbr2, Parp2, Rgs4, Ccnb1, Mapk14, Gsk3a, Mapk1, G6pd2) and cardiovascular (Tbx2, Smo, Tgfbr1, Mdm2, Zfpm2, Robo1, Smad7, Parva, Tbx20, Tgfbr2, Smad6, Fzd1, Zfpm1, Trp53, Sox11) developments. In certain embodiments, the proteins and/or peptides can treat, for example, hypertension and cardiomyopathy.

In certain embodiments, the subject compositions can be used in methods of treating diabetes. In certain embodiments, the proteins and/or peptides of the subject invention induce differentially expressed genes that are significantly enriched in biological functions and processes that modulate positive regulation of insulin secretion involved in cellular response to glucose stimulus (Arrb1, Ppp3cb, Camk2n1, Gpr68), insulin receptor signaling pathway (Tsc2, Ankrd26, Rarres2, Pip4k2b, Rps6kb1, Mstn, Pip4k2a, Src, Zbtb7b, Ybx1, Sesn3, Ccnd3, Ncoa5, Pik3r1, Sorl1, Prkaa1, Socs3, Gsk3a), insulin signaling pathway (Hk2, Araf, Tsc2, Prkar2b, Gys1, Crkl, Ppp1cb, Crk, Mknk2, Shc2, Rps6kb1, Prkar1a, Pygl, Pdpk1, Hras, Hk3, Pik3r3, Mknk1, Rheb, Add1, Prkab1, Kras, Pde3b, Pik3cb, Cbl, Sos2, Phkb, Pik3cd, Pik3r1, Mapk10, Prkaa1, Socs3, Irs4, Flot1, Mapk3, Mapk1), positive regulation of insulin secretion (Arrb1, Ppp3cb, Camk2n1, Gpr68, Gja1, Bglap2), regulation of glucose metabolic (Kat2b, Slc35b4, Grb10, Usp7, Mtor, Bckdk, Ogt, Foxk2) and catabolic (Hk1, Foxk2) processes, cellular glucose homeostasis (Slc39a14, Pck1, Ogt, Hk1, Foxk2).

In certain embodiments, the subject compositions can be used in methods of wound healing. In certain embodiments, the proteins and/or peptides of the subject invention induce differentially expressed genes that significantly enrich positive regulation of wounding (Cldn13, Dmtn, Enpp4, Vegfb, Mtor, Foxc2) and vascular wound (Vegfb, Foxc2) healings, angiogenesis involved in wound healing (Ndnf, Kdr), regulation of inflammatory response to wounding (Git1, Alox5, Cd24a). In certain embodiments, the proteins and/or peptides of the subject invention enhance macrophage polarization, which also plays important roles in wound healing.

In certain embodiments, the subject compositions can be used in methods of post-menopausal syndrome treatments. In certain embodiments, the proteins and/or peptides of the subject invention suppress Ovariectomized (OVX)-induced osteoporosis, which is one of the models for post-menopausal syndrome. In certain embodiments, the proteins and/or peptides induce differentially expressed genes that are significantly enriched in positive regulation of intracellular estrogen receptor signaling pathway and response to estrogen. In certain embodiments, the proteins and/or peptides can treat some post-menopausal syndromes, such as, for example osteoporosis.

In certain embodiments, the subject compositions can be used in methods of infertility treatments. In certain embodiments, the proteins and/or peptides induced differentially expressed genes that are significantly enriched in oocyte differentiation (Atm, Bcl2) and maturation (Fbxo5, Rps6ka2, Ereg, Ccnb1, Foxo3), spermatogenesis (Axl, Bax, Crkl, Sfmbt1, Bcl2l1, Herc4, Sgpl1, Ube2b, Apob, Tlk2, Vipas39, Mettl3, Bcl6, Bsg, Bag6, Prdx4, Prnd, Bcl2l11, S100a11, Celf3, Spaca1, Pum1, Ndc1, Inpp5b, Nek1, Mlh1, Wdr48, Ythdc1, Siah1a, Parp11, Mical2, Arid4b, Tdrd6, Slco4c1, Dmxl2, Katnal1, Setx, Cfap43, Zfp41, Catsperg2, Prss43, Cdyl, Hspa2, H3f3a, Cyp26b1, Nr6a1, Dazap1, Btbd18, Mfsd14a, Fancf, Syne1), sperm mitochondrion organization (Ddhd1, Vps13a, Nectin2, Mfsd14a), regulation of fertilization (Cfap69), developmental process involved in reproduction (Pparg, Wdr77, Axl, Tgfb1, Bax, Stat5a, Ptpn6, Prlr, Celf1, Crkl, Sfmbt1, Tgfbr1, Bcl2l1, Arid1a, Map3k4, Ptgis, Med1, Lhx9, Ubb, Fbxo5, Rspo3, Kitl, Herc4, Sgpl1, Ube2b, Rps6kb1, Myocd, Apob, Tlk2, Stat5b, Vipas39, Hif1a, Zfp36l1, Ctsl, Mettl3, Bcl6, Dlg1, Tnk2, Bsg, Rps6ka2, Birc6, Epas1, Msh2, C3, Bag6, Prdx3, Prdx4, Ube3a, Plk4, Serpine2, Asb1, Lhx4, Nr5a1, Sp3, Prnd, Bcl2l11, Src, Ncoa3, Smad9, S100a11, Celf3, Spaca1, Pum1, Ndc1, Pithd1, Inpp5b, Ereg, Tmf1, Ddias, Tial1, Flna, Nek1, Irx5, Fem1b, Mlh1, Wdr48, Fanca, Irf2bpl, Atm, Hectd1, Ythdc1, Siah1a, Paqr7, Icam1, Serpine1, Pank2, Parp11, Mical2, Ncoa6, Taf4, Abhd2, Arid4b, Tdrd6, Slco4c1, Dmxl2, Katnal1, Ccnb1, Snai1, Serpinb6b, Greb1l, Setx, Adam6a, Cfap43, Ermp1, Zfp41, Pygo2, Foxo3, Catsperg2, Tifab, Foxc1, Lin28a, Junb, Socs3, Mapk14, Sec23ip, Dach1, Cebpb, Bcl2, Bak1, Prss43, Rsl1, Cdyl, Hspa2, H3f3a, Mapk3, Mapk1, Cyp26b1, Nr6a1, Dazap1, Etnk2, Ccnf, Fsip2, Syna, Xist, Btbd18, Mfsd14a, Fancf, Syne1), cellular process involved in reproduction in multicellular organism (Wdr77, Tgfb1, Bax, Celf1, Tgfbr1, Bcl2l1, Zmiz1, Ubb, Fbxo5, Kitl, Ube2b, Rps6kb1, Top2a, Mettl3, Tnk2, Rps6ka2, Msh2, Src, Spaca1, Pithd1, Ereg, Tmf1, Etv6, Ddias, Tial1, Ccnb2, Mlha, Fanca, Atm, Siah1a, Paqr7, Pank2, Ttk, Abhd2, Tdrd6, Ccnb1, Cfap43, Spesp1, Pygo2, Foxo3, Foxc1, Lin28a, Sec23ip, Bcl2, Prss43, Cdyl, Hspa2, Prmt7, H3f3a, Cyp26b1, Zbtb16, Fsip2, Btbd18, Mfsd14a), regulation of blastocyst development (Ttll4, Agbl4), inner cell mass cell proliferation (Setdb1, Prpf19, Gins4, Chek1, Ncapg2, Palb2), in utero embryonic development (Ccm2, Smo, Keap1, Dll3, Tgfbr1, Bcl2l1, Zmiz1, Cr1l, Med1, H13, Cops3, Ube2b, Apob, Gna13, Kat2a, Gli3, Dab2, Zfpm2, Msh2, Mib1, Pdgfrb, Zfand5, Sp3, Traf6, Bcl2l11, Dhx35, Mbnl1, Cpt2, Ybx1, Inpp5b, Add1, Grin2b, Atp11a, Ccnb2, Tgfbr2, Npat, Epn1, Ankrd11, Ythdc1, Gpi1, Wdtc1, Sec24d, Sec24c, Ccnb1, Maff Ubr3, Palb2, Foxc1, Slc39a1, Junb, Klf2, Trp53, Zbtb18, Uty, Etnk2, Atf7), positive regulation of uterine smooth muscle contraction, response to progesterone (Tgfb1, Ube3a, Src, Wbp2, Abhd2, Mbp), progesterone receptor signaling pathway (Ube3a, Src, Klf9, Wbp2, Yap1) and response to estrogen (Adcy3, Adcy7, Bcl2, Creb1, Creb3l1, Fos, Gnai1, Gp9, Hras, Hspa2, Jun, Kcnj6, Kras, Krt14, Krt24, Krt26, Mapk1, Mapk3, Mmp2, Ncoa3, Oprm1, Pik3cb, Pik3cd, Pik3r1, Pik3r3, Plcb2, Shc1, Shc2, Sos1, Sos2, Src).

In certain embodiments, the subject compositions can be used in methods of treatment of hematopoietic diseases. In certain embodiments, the proteins and/or peptides induce differentially expressed genes that are significantly enriched in regulation of hematopoietic or lymphoid organ development (Tgfb1, Stat5a, Cnn2, Crkl, Pknox1, Tgfbr1, Med1, Ikzf1, Kitl, Psen1, Sgpl1, Mknk2, Id2, Klf11, Stat5b, Hif1a, Tnfsf11, Epas1, Pdgfrb, Csf1r, Men1, Nfkb2, Slc40a1, Tnfrsf11a, Ptprc, Sp3, Bcl2l11, Slc46a2, Ddias, Ccnb2, Tgfbr2, Zbtb1, Lyl1, Atm, Ttc7, Ccr7, Picalm, Cdk13, Lyn, Sh2b3, Onecut1, Zfp36l2, Wbp1l, Samd9l, Cxcr5, Selpig, Ccr2, Zfpm1, Bcl3, Bcl2, Trp53, Mapk3, Mapk1, Zbtb16, Ceacam1), hematopoietic progenitor (Ap2a2, Nfe2l2, Eif2ak2, Zbtb1, Sos2, Ankle1, Foxc1) and stem ((Ap2a2, Nfe2l2, Eif2ak2, Foxc1) cell differentiation, hematopoietic stem cell homeostasis (Tcirg1, Glis2, Fstl1, Arhgef5, Ubap2l, Emcn, Ext1), regulation of hematopoietic stem cells proliferation (Kitl, Eif2ak2, Cxcl1, Atxn1l), positive regulation of angiogenesis (Itgb2, Hk2, Aqp1, Notch4, Nfe2l2, Ptgis, Nr2e1, Xbp1, Hif1a, Sema5a, Cyp1b1, C3, Itgb8, Cxcr2, Jak1, Add1, Ctsh, Tgfbr2, Ccr3, Btg1, Hspb6, Lrg1, Serpine1, Pik3cd, Agtr1a, C5ar1, Sphk1, Chil1), positive regulation of blood vessel endothelial cell proliferation involved in sprouting angiogenesis (Hmox1, Vegfa, Ppp1r16b, Aplnr, Agtr1a), positive regulation of cell migration involved in sprouting angiogenesis (Hdac9, Hmox1, Akt3, Plk2, Vegfa, Anxa1, Srpx2, Foxc2, Kdr), vasculogenesis (Ccm2, Foxm1, Smo, Tgfb1, Epha2, Zmiz1, Sgpl1, Myocd, Zfp36l1, Zfpm2, Itgb8, Spred1, Tbx20, Tgfbr2, Gjc1, Junb), vasculogenesis involved in coronary vascular morphogenesis (Epor, Fgfr2). In certain embodiments, the proteins and/or peptides can also protect RBCs against hypotonic-induced hemolysis. In certain embodiments, the proteins and/or peptides can therefore be a therapeutic intervention against blood related diseases, such as, for example, hemolytic anemia.

### Materials and Methods

### 5.1 Materials and Animals

### 5.1.1. HKUOT-S2 Isolation Equipment, Materials, and Reagents

The high-speed refrigerated centrifuge from HITACHI (hicmac CR21G II) and the AKTAexplorer Purifier from GE Healthcare were respectively provided by the Department of Surgery and the School of Chinese Medicine, The University of Hong Kong (HKU). The HKUOT-S2 isolation columns namely Hiprep DEAE FF 16/10 (Cat #28-9365-41), Hiprep Phenyl FF (high Sub) 16/10 (Cat #28-9365-45), and Superdex 75 Increase, 10/300 GL (Cat #29-1487-21) were purchased from GE Healthcare, USA. The following reagents with catalogue numbers and supplying companies in parenthesis were also purchased for HKUOT-S2 extraction and purification: acetic acid glacial (Cat #3839-2.5L, DUKSAN), β-mercaptoethanol (Cat #A2008-250ML, Biomatik), 99%, ultrapure ammonium sulfate (Cat #J11254-A1, Thermo Scientific), SnakeSkin Dialysis Tubing (Cat #68700, Thermo Scientific), ultrapure Sodium chloride, (Cat #J21618-A1, Thermo Scientific), Tris (Cat #15504-020, Invitrogen), Na2HPO4 (Cat #3153/500G, Tocris), ovalbumin (Cat #A5253, Sigma), and BSA (Cat #A-420-250, GoldBio), Aprotinin (Cat #4139/10, Tocris).

### 5.1.2. Surgical Equipment and Reagents

The Vicryl suture 6-0, (Cat #W9981) and Mersilk Suture 5-0 (W500) were bought from Ethicon. Heparin sodium Inj. (Cat #HK-28227) was obtained from B. Braun Melsungen, betadine was provided by department of Orthopedics and Traumatology, HKU.

### 5.1.3. Animal License, and Ethical Approval

Animal license [Ref No.: (20-1000) in DH/HT&A/8/2/3 Pt.14] to conduct this experiment using mice was granted by the Licensing Office of the Department of Health, Hong Kong Government. Ethical approval (CULATR 5502-20) to handle the animals under this research was obtained from the HKU Ethics Committee, Committee on the Use of Live Animals in Teaching and Research (CULATR).

### 5.1.4. Acute Toxicity Study Equipment and Reagents

The microhematocrit centrifuge (Cat #SKU: VQ5578(a)) and glass capillary tubes (Cat #87002-161) were purchased from Iris Sample Processing, and VITREX respectively. The clinical chemistry analyzer (BS-230) was purchased from Mindray by the School of Chinese Medicine, HKU. The cuvettes for BS-230 (Cat #HTM 115-037543-00), ALP (Cat #HTM 105-000816-00a), ALT (Cat #HTM 105-000814-00a), AST (Cat #HTM 105-000815-00a), CREA-S(Cat #HTM 105-004614-00), UREA (Cat #HTM 105-000824-00a), multi-sera calibrator (Cat #HTM 105-001144-00a) and CD80 detergent (Cat #HTM 105-000748-00) were purchased from the Healthpro Technology Co. Ltd.

### 5.1.5. Cell Lines

The mouse M0 macrophage, RAW264.7 cells (Cat #TIB-74), mouse preosteoblast cells, MC3T3-1E subclone 4 (Cat #CRL-2593), and human pro-monocyte, U937 cells (Cat #CRL-1593.2) lines were purchased from ATCC. The human turbinate mesenchymal stromal cell line (hMSCs), harvested by Kwon et al., ^{[72]} were used.

### 5.1.6. In Vitro Cell Culture Reagents and Media

The DMEM low glucose medium (Cat #11885076), DMEM high glucose medium (Cat #12100-046), RMPI medium 1640 (1X) (Cat #a10491-01), fetal bovine serum (FBS) (Cat #10270-106, Gibco), penicillin-streptomycin (P/S) (Cat #15140122), L-glutamine (Cat #21051-024), 0.25 ug/ml amphotericin B (Cat #15290-018), 0.25% trypsin-EDTA (Cat #25200-056) and 10× phosphate buffered saline (PBS) (Cat #70011-044) were purchased from Gibco, Thermo Scientific. The NaHCO₃ (S5761-1KG), HEPES (H4034-1KG) and MTT (M5655-100MG) were bought from Sigma.

### 5.1.7. Cell Polarization and Differentiation Compounds and Chemicals

Lipopolysaccharide (LPS) (Cat #L4391-1MG), dexamethasone (Dex) (Cat #D4902-25MG), L-Ascorbic acid (Asc) (Cat #A4544-100G), β-Glycerophosphate disodium salt hydrate (β-Gly) (Cat #G9422-50G), Phorbol 12-myristate 13-acetate (PMA) (Cat #79346-1MG) were purchased from Sigma. Recombinant mouse IL-4 (Cat #404 ML) and human IL-4 (Cat #204-IL) were bought from R&D System.

### 5.2. Extraction and Isolation of HKUOT-S2 Protein

The Dioscorea opposita Thunb tubers were bought from the wet markets in Hong Kong. The extraction and isolation of the HKUOT-S2 was carried out according to previously published protocol with some minor modifications. ^{[[38]]}Briefly, the weighed (g) tubers were peeled. 2 parts by volume (ml) of cold extraction buffer consisting of 5% acetic acid, and 0.1% β-mercaptoethanol was added to 1 part by weight (g) of the tubers and blended in the hood. The resultant homogenized mixture was subjected to magnetic stirring for 3 hr at 4° C. The homogenate was supersaturated with 80% (NH₄)₂SO₄ and stirred overnight at 4° C. The cold homogenates were subjected to centrifugation at 4° C., 14000 rpm for 1 hr using the high-speed refrigerated centrifuge, to precipitate the crude protein mixture. The crude protein mixture was resuspended in cold Milli-Q water, distributed in Snakeskin dialysis tubing (7000 MWCO), subjected to dialysis in Milli-Q H2O overnight at 4° C. and high-speed refrigerated centrifugation at 14000 rpm for 2 hrs. The collected dialyzed crude protein mixture was processed for downstream successive fast protein liquid chromatography (FPLC) purifications. To isolate the novel HKUOT-S2 protein, the dialyzed crude protein mixture was subjected to successively different column purifications. First the dialyzed crude protein was lyophilized, dissolved in 100 mM tris buffer, and subjected to ion exchange chromatography using HiPrep 16/10 DEAE FF column (GE Healthcare, Sweden), buffer A (100 mM Tris) and buffer B (1 M NaCl+100 mM Tris, gradient (0-45%). The osteogenic fraction D3 (HKUOT-D3), obtained from the crude protein extracts, was then subjected to dialysis and lyophilization. Concentrated solution of HKUOT-D3 in buffer A (Milli-Q H₂O) was added to buffer B (10 mM Na₂HPO4+1 M (NH₄)₂SO₄) in 1:1 ratio. The reconstituted HKUOT-D3 solution was subjected to hydrophobic interaction chromatography using HiPrep Phenyl FF (high sub) 16/10 column (GE Healthcare, Sweden), buffer A and gradient buffer B (30-0%). The dialyzed and lyophilized osteogenic peak P1 (HKUOT-P1), obtained from HKUOT-D3 and dissolved in buffer A (50 mM Na₂HPO₄+150 mM NaCl), was subjected to size-exclusion chromatography (SEC) using Superdex 75 Increase 10/300 GL column (GE Healthcare, Sweden) calibrated with known molecular weight markers [aprotinin (6.5 kDa), ovalbumin (44.287 kDa), and BSA (66 kDa)] and buffer A. The SEC gave rise to pure fraction S2 (HKUOT-S2 protein). All the columns were mounted and operated on an AKTA Purifier (GE Healthcare, Sweden) FPLC system according to the specific manufacturer's recommendations. The dialyzed and lyophilized osteogenic HKUOT-S2 was stored at −80° C. for downstream applications.

### 5.3. Characterization of HKUOT-S2 Protein

### 5.3.1. Prediction of the Molecular Weight of HKUOT-S2 Using Mathematical Model

The molecular weight (MW) of HKUOT-S2 was predicted mathematically using the standard curve of the calibrated Superdex 75 Increase 10/300 GL column information and Kav equation.

According to the Kav equation formular,

$$Kav ( HKUOT - S 2 ) = \frac{( Ve - V 0 )}{( Vc - V 0 )}$$

Where Ve=elution volume (11.232 ml), V0=void volume (8 ml) and Vc=column volume (24 ml)

$$Kav ( HKUOT - S 2 ) = \frac{( 11.232 - 8 ) ml}{( 24 - 8 ) ml} = 0.202$$

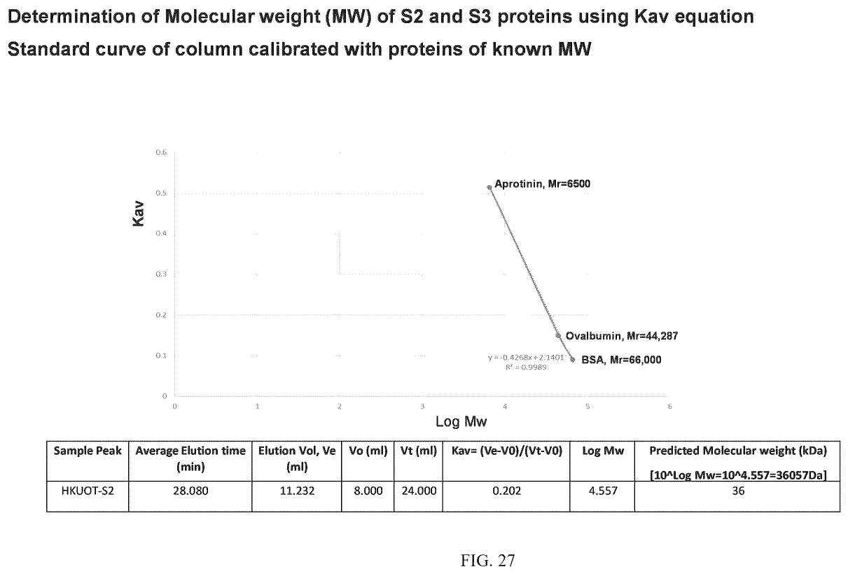

Standard curve of the calibrated Superdex 75 Increase 10/300 GL column is shown in FIG. 27.

From the standard curve, Kav=y and Log Mw=X.

From the Standard curve linear equation, Kav=−0.4268(Log MW)+2.1401

$$log Mw ( HKUOT - S 2 ) = \frac{( 2.1401 - 0.2028 )}{0.4268} = 4 \text{.5410}$$

Mw (HKUOT-S2)=104.5410=34753 Da=34.8 kDa

From the column calibrated standard curve of proteins of known MW, Kay values are inversely proportional to protein MW

$$Kav ( HKUOT - S 2 ) α \frac{1}{MW ( HKUOT - S 2 )}$$

$$Kav ( HKUOT - S 2 ) = \frac{K}{MW ( HKUOT - S 2 )}$$

Where K=constant=Kav (known protein)*MW (known protein)

$$Mw ( HKUOT - S 2 ) = \frac{Kav ( known protein ) * MW ( known protein )}{Kav ( HKUOT - S 2 )}$$

Taking ovalbumin as reference protein with known Kay of 0.15 and Mw of 44287 Da, together with Kay (HKUOT-S2) of 0.202, then

$$Mw ( HKUOT - S 2 ) = \frac{0.15 * 44287}{K 0.202} = 32886 Da = 32.9 kDa$$

The MW(HKUOT-S2) therefore, ranges from 32.9-34.8 kDa.

### 5.3.2. HKUOT-S2 MW Determination by Silver Staining

The HKUOT-P1 and HKUOT-S2 proteins were subjected to electrophoresis using 15% SDS-PAGE and silver staining using the Pierce™ Silver Stain Kit (Cat #24612, Thermo Scientific) according to the manufacturer's protocol. The silver staining indicated single band of HKUOT-S2 close to 32.9 kDa and double bands of HKUOT-P1 between 32 and 35 kDA.

### 5.3.3. HKUOT-S2 Molecular Mass Determination and Identification by Mass Spectrometry

The HKUOT-S2 protein bands were incised for successive MALDI-MS and high-resolution LC-MS/MS analyses at the LKS Faculty of Medicine, Proteomics and Metabolomics Core Facility (PMcore), Centre for PanorOmic Sciences (CPOS), the University of Hong Kong. The MALDI-MS confirmed the MW of HKUOT-S2 to be around 32 kDa consistent with the MW determined by both Mathematical model and silver staining techniques. For LC-MS/MS analysis, in-gel protein trypsinization was performed to digest the HKUOT-S2 protein into peptides. The resultant peptides were sequentially extracted from the gel using 50% ACN/5% FA and 100% ACN. The pooled peptides were speedvac dried, desalted using C18 StageTips and processed for LC-MS/MS analysis. The eluted peptides were analyzed with Dionex Ultimate3000 nanoRSLC system coupled to Orbitrap Fusion Tribid Lumos mass spectrometer (Thermo Fisher) and separated using commercial C18 column coupled to a NanoTrap column (Thermo Fisher). Full mass spectrometer (MS) survey scan resolution was set to 120 000 with an automatic gain control (AGC) target value of 4×105, maximum ion injection time (IT) of 30 ms, and for scan range of 400-1500 m/z. Spectra were obtained at 30000 MS2 resolution with AGC target of 5×104 and maximum IT of 80 ms, 1.6 m/z isolation width, and normalized collisional energy of 30. The raw MS data were processed using MaxQuant 1.6.14.0 and searched against Dioscorea UniProt FASTA database (June 2020) containing 18,477 entries. Confident proteins were identified using a target-decoy approach with a reversed database, strict false-discovery rate 1% at peptide and peptide spectrum matches (PSMs) level.

### 5.3.4. HKUOT-S2 De Novo Sequencing

Targeted LC-MS/MS de novo sequence was performed to identify the HKUOT-S2. The peptide obtained by the LC-MS/MS were matched against the NCBInr Dioscorea genus protein database. NCBI protein basic local alignment search tool (BlastP) results with E-value<0.001, were considered significant and used as inclusive criteria for alignments of high quality. The severally identified de novo peptides of HKUOT-S2 protein had no significant match with the NCBInr Dioscorea genus protein database. BlastP of HKUOT-S2 de novo peptide sequences against Dioscorea spp also yielded no significant peptide sequence matches. The HKUOT-S2 is potentially a novel candidate protein with unique de novo peptide sequences. PEAKS Studio X-Pro analysis of these unique de novo sequences revealed the peptide intensities and m/z ratios.

### 5.3.5. HKUOT-S2 N-Terminal Peptide Sequencing

N-terminal Sequencing of HKUOT-S2 using Edman degradation was done by the Creative Proteomics Company, USA. The N-terminal sequencing of HKUOT-S2 was performed on the polyvinylidene fluoride (PVDF) membrane. The analysis was done on an ABI Procise 494HT (Thermo Fisher). The N-terminal amino acid residues of HKUOT-S2 were cleaved off one at a time and identified by chromatography. The Edman degradation chemistry involved firstly coupling PITC reagent to the N-terminal amino group in alkaline conditions followed by the cleavage of the N-terminal residue under acidic conditions. The PITC coupled residue transferred into a flask, was then converted to a PTH-residue and identified by HPLC chromatography. The next cycle was then started for identification of the next N-terminal residue. N-terminal Sequencing of HKUOT-S2 by Edman degradation chemistry revealed unique N-terminal sequences.

### 5.4. In Vivo Studies

A total of 96 C57BL/6 male mice were used in these in vivo studies. Eight to ten-week-old mice, weighing between 22-25 g, obtained from the HKU Laboratory Animal Unit (LAU) were provided with a good temperature, clean shelter, food water, good ventilation and treated humanely. The mice were housed in standard open top cages at HKU Center for Comparative Medicine Research (CCMR) housing unit. The mice, upon delivery, were allowed to acclimatize to their new environment for one week prior to the commencement of the experiments.

### 5.4.1. Effects of HKUOT-S2 Treatments on Hemolysis

A hemolytic test was carried out on mice red blood cells (RBCs) to determine the anti-hemolytic effect of HKUOT-S2 protein. ^{[47]}Briefly, 1 mL of heparinized fresh whole blood samples were collected from euthanized C57BL/6 male mice by cardiac puncture. The blood samples were centrifuged at 1,500×g for 5 min and the resultant RBCs pellets were washed 4× with 1 ml of 1×PBS. After the last centrifugation, 200 ul of 6.0×10⁷ RBCs suspension in 0.5 ml Eppendorf tubes were treated with 1×PBS (negative control), 1× triton-X-100 (positive control), 3.13-200 μg ml⁻¹ of HKUOT-S2, diclofenac, melittin, and piscidin. The closed and parafilm sealed tubes were incubated at 37° C. for 1 h followed by centrifugation at 1,500 rpm for 5 min. 50 ul of the sample supernatants were carefully transferred in triplicates into the 96-well plates. The optical densities of the samples were measured at 540 nm (OD540) using the microplate reader.

$$Hemolysis ( \% ) = \frac{( corrected ( OD 540 of treatment - OD 540 of 1 XPBS control )}{corrected OD 540 of 1 XPBS control} * 100$$

### 5.4.2. Effects of HKUOT-S2 Treatments on Hypotonic-Induced Hemolysis

A hypotonic solution-induced hemolysis test was carried out according to the previously published protocol, ^{[73]}to investigate hemolytic inhibitory effects of HKUOT-S2. Briefly, 10 ul of the stock RBCs suspension was added to 1 ml hypotonic solution (0.45% NaCl, pH 7.4) containing 0-2000 μg ml⁻¹ HKUOT-S2 protein, BSA, diclofenac sodium and piscidin. The 10 ul RBCs suspensions in 1 ml of isotonic (0.9% NaCl, pH 7.4) and hypotonic (0.45% NaCl, pH 7.4) solutions without any drugs were used as the negative and positive controls respectively. The mixtures were incubated for 1 hr at room temperature and centrifuged for 10 min at 1000 rpm. 50 ul of the sample supernatants were carefully transferred in triplicates into the 96-well plates. The hemoglobin content of the supernatant was measured spectrophotometrically at 560 nm (OD560). The percentage of hypotonic-induced hemolysis inhibition was calculated as:

$$Inhibition of hemolysis ( \% ) = [ \frac{corrected ( OD β - OD α )}{( corrected ( OD ϒ - OD α )} ] * 100$$

Where, ODα=OD560 of negative control (0.9% NaCl)
ODβ=OD560 of treatment samples in 0.45% saline solution
ODγ=OD560 of positive control (0.45% NaCl)

### 5.4.3. Acute Toxicity Studies of HKUOT-S2 Treatments Toxic In Vivo

The acute toxicity of HKUOT-S2 treatments were evaluated in mice in vivo. The initial body weights and physical appearances of the mice were noted prior to the HKUOT-S2 treatments. The mice were treated with 1.09-4.36 mg kg⁻¹ HKUOT-S2 treatments thrice per week with the sham control treated with only 1×PBS (pH 7.4). On the first day of HKUOT-S2 treatments, the mice were observed for any toxic signs in the first 30 mins after drug administration and then observed every 4 hrs, followed by daily observation for toxic signs till the end of the experiment (week 4). The changes in body weights (g) of the mice were also measured weekly throughout the study period. The mice were euthanized with 100 mg kg⁻¹ pentobarbital. The blood samples were collected via cardiac puncture for biochemical analysis, the liver and kidney were harvested surgically and frozen for RNA extraction by qPCR and histopathological analyses. As HKUOT-S2 treatments had no toxic effects in vivo in the non-surgical mice, the same experimental procedures, and evaluations of the acute toxicity study of HKUOT-S2 treatments were also repeated in the bone defect mouse model.

### 5.4.4. Effects of HKUOT-S2 Treatments on Mouse Hematocrit

A manual microhematocrit test was performed to evaluate the effects of HKUOT-S2 treatments on RBCs levels in vivo. Approximately, 9 μl of venous blood was collected from the saphenous vein of the sham control and HKUOT-S2 treated mice into heparinized microhematocrit capillary tubes. The tubes were sealed with a modelling clay and centrifuged for 2 minutes at 3000 g using StatSpin veterinary centrifuge. The hematocrit was determined manually using microhematocrit capillary tube reader (StatSpin). The measurements were double confirmed with 30 cm ruler.

### 5.4.5. Effects of HKUOT-S2 Treatments on Serum Biochemistry

Clinical biochemical analysis was performed to evaluate the effects of HKUOT-S2 treatments on liver and kidney functions in vivo. Non-heparinized whole blood samples were collected by cardiac puncture from the sham control and HKUOT-S2 treated mice into the 1.5 ml Eppendorf tubes. The collected blood samples were kept at 4° C. for 30 minutes to allow blood clotting. The clotted samples were centrifuged for 10 mins at 1000 rpm. The sera were gently collected and aliquoted into new 0.2 ml Eppendorf tubes. Triplicates of 70 ul sera in cuvettes were subjected to biochemical analyses to determine the serum alanine aminotransferase (ALT), alkaline phosphatase (ALP), aspartate aminotransferase (AST), blood urea nitrogen (urea), and creatinine (CRE) levels using the automated clinical chemistry analyzer (Mindray Bs-230).

### 5.4.6. Effects of HKUOT-S2 Treatments on Bone Defect Healing In Vivo

All animal surgical procedures and protocols were carried out strictly according to the methods approved by the HKU Committee on the Use of Live Animals in Teaching and Research (CULATR). The mice were served drinking water containing analgesic Meloxicam (1 mg kg⁻¹) for two days prior to and 5 days after surgery. Prior to the surgical procedure, the mice were anaesthetized through intraperitoneal (i.p.) injection of 100 mg kg⁻¹ ketamine hydrochloride and 4 mg kg⁻¹ xylazine. The hairs on skin of the dorsal right femurs were shaved. The mice, placed on the heat pad, were given subcutaneous injection of 1 ml warmed normal saline solution. The shaved skins were cleansed alternatively with betadine and 70% ethanol for 4 times. Sharp sterile razor blades were used to cut the superficial and subcutaneous skins and muscles layers along the femurs to expose the femur. A circular-through bone defects were created on the right distal femurs, 2 mm above the epiphyseal plates (diaphysis). To create the bone defects, a sterile 21 G needle, held in the hand with minimal but enough force applied, was used to pierce through the surgically exposed bone carefully and perpendicularly, from the anterior surface down to the opposite posterior surface to drill a 0.9 mm diameter circular holes. The bone defect sites were irrigated with 1 ml warmed 1×PBS to flash out any bone debris therein. The wounds were closed by systematically suturing the muscles with absorbable Vicryl suture 6-0, subcutaneous and superficial skin layers with Mersilk Sutures. Immediately after surgery, the mice were given subcutaneous injection of antibiotic enrofloxacin (5 mg kg⁻¹) and an analgesic buprenorphine (0.05 mg kg⁻¹). Thereafter, the analgesic buprenorphine was administered twice a day for three days after the surgery. The mice were randomly assigned into four experimental groups namely the sham control, the 1.09, 2.36 and 4.36 mg kg⁻¹ HKUOT-S2 treatment groups. Each experimental group contained 8 mice (n=8). The mice were given subcutaneous injections of 200 μl HKUOT-S2 solutions for the HKUOT-S2 treatment groups and 200 μl of 1×PBS for the sham controls above the bone defect sites immediately after surgery and thrice per week for 4 weeks. At the experimental endpoints, blood samples were collected from the sham control and HKUOT-S2 treated mice by cardiac puncture. The blood samples were processed for downstream applications such as hematocrit test, clinical biochemistry, ELISA, and western blot analyses. Both surgical and non-surgical femurs, liver and kidneys were also harvested and processed for other applications such as qPCR, immunostaining, histological and RNA-seq analysis.

5.4.7. Micro-Computed Tomography (pCT) Scan Analysis of the Bone Defects.

The bone defects sites of the mice under anesthesia were subjected to μCT scanning immediately after the surgery using the Skyscan (Cat #1076, Bruker) followed by weekly μCT scans for 1 month to monitor the HKUOT-S2-induced bone defect repairs in vivo. The X-ray images obtained from the scanning of the bones were reconstructed into 2D images using the software (NRecon) provided by the Skyscan Company. The CTvox software was used to reconstruct the 3-D-images of the sham control and HKUOT-S2 treated femurs. To determine the mineral densities of the bone samples, the same μCT settings for the bone tissue scanning were used to scan two phantoms (2 mm in diameter) of calcium hydroxyapatite (CaHA) rods of known densities (0.25 and 0.75 gcm⁻³). The determined attenuation coefficients (ACs) for the 0.25 and 0.75 gcm⁻³ phantoms were 0.02616 and 0.05773 mm⁻¹ respectively. The known densities of the CaHA together with the corresponding ACs were used to calibrate the BMD and TMD of the μCT scan bones using the CTAn software. The CTAn software was then used to evaluate the effects of HKUOT-S2 treatments on bone parameters such as BV/TV, BMD, TMD, Tb.th, Tb. N, Tb. Sp, BS/BV and BS/TV during bone defect repairs.

### 5.4.8. Histological Processing of the Undecalcified Bone Tissue

The femurs from the sham control and HKUOT-S2 treated mice were harvested at the experimental endpoint. The harvested bone tissues were immediately transferred into 10% neutral buffered formalin (10% NBF) for 24 hrs fixation. Afterwards, the bones were dehydrated in ascending grade of ethanol (70-100%), cleared in a xylene, and embedded sequentially in methyl methacrylatel (MMAI), methyl methacrylate II (MMAI+20 g dibenzoyl peroxide), and methyl methacrylate III (MMA I+20 g dibenzoyl peroxide+250 ml dibutyl phthalate). The embedded samples were sectioned into 200 μm and further ground or polished into thinner sections between 50-70 μm. Selected sections were subjected to Giemsa staining.

### 5.4.9. Histological Processing of the Decalcified Bone Tissue

The 10% NBF fixed femurs, harvested from the sham control and HKUOT-S2 treated mice, were decalcified in 0.5M ethylenediaminetetraacetic acid (EDTA, Sigma-Aldrich) for 48 hrs. The decalcified bones were washed under running water for 30 minutes, dehydrated in ascending grade of ethanol (70%-100%), and subsequently cleared in xylene. The samples were then processed and embedded in paraffin wax for downstream staining and analyses.

### 5.4.10. Fluorochrome Labeling

Fluorochrome labeling was employed to evaluate new bone formation at the bone defect sites during HKUOT-S2 treatments. Briefly, 10 mg kg⁻¹ Calcein green (Cat #C0875-5G, Sigma-Aldrich) and 90 mg kg⁻¹ xylenol orange (Cat #X-0127, Sigma-Aldrich) were prepared in 2% NaHCO₃. Calcein green was administered intraperitoneally on (day 19) post-surgery. The xylenol orange was also given intraperitoneally three days prior to euthanasia. The harvested undecalcified bone tissues were embedded in MMA and processed for inverted confocal fluorescence microscopy. The inverted confocal fluorescence microscope, Carl Zeisis (LSM900) equipped with the ZEISS (ZEN 3.4, blue edition) software was used to acquire and quantify the fluorochrome intensities in vivo.

### 5.4.11. Hematoxylin and Eosin (H &E) Staining of the Decalcified Bones

The rotary microtome was used to section 4 μm of the paraffin embedded femurs. The paraffin sections were placed on adhesive tissue slides for histological staining. For H &E staining, the bone tissues on the slides were deparaffinized with xylene, rehydrated in descending grade of ethanol (100-70%) and water. The rehydrated bone tissues were stained with Harris hematoxylin modified solution (HHS32-1L, Sigma-Aldrich) for 2 minutes, washed in water, differentiated in 70% ethanol containing 1% HCl, blued in 0.1% NH4OH solution and counterstained with eosin (E03210-7G, UNI-Chem). The tissues were subjected to serial dehydration in 70-100% ethanol, cleared in xylene, dried, and covered with cover slips using mounting medium, Pertex (Cat #41-40011-00, Medite).

### 5.4.12. Giemsa Staining of MMA Embedded Bone Sections

The 50-70 μm sections of the MMA embedded bone tissues were slightly de-plasticized in 2% HNO₃(aq) and immersed in Xylene for 2 minutes. The bone tissues were rehydrated in graded descending order of ethanol (100-70%) and ddH₂O. The tissues were submerged in 10× Giemsa stain for 3-4 minutes, washed in ddH₂O for 2 minutes, air-dried and covered with cover slips using mounting medium.

### 5.4.13. Trichrome Staining of Paraffin Embedded Bone Sections

The 4 μm sections of the paraffin embedded femur tissues were deparaffinized, rehydrated, stained with prepared Weigert Iron hematoxylin, washed with ddH₂O and 1% acetic acid and incubated in azophloxine solution (Cat #100485/1, Sigma-Aldrich) for 10 minutes followed by washing in 1% acetic acid for 30 seconds. The tissues were submerged in tungstophosphoric acid orange G solution (Cat #100485/2, Sigma-Aldrich) for 1 minute, and light green SF solution (Cat #100485/3, Sigma-Aldrich) with in-between washing with 1% acetic acid. The bone tissues were dehydrated in ascending order of ethanol, cleared in xylene, air-dried, and mounted with cover slips using mounting medium. All the histologically stained bone tissues were viewed under Eclipse 80i compound fluoresce Microscope (Nikon) equipped with the NIS-Elements software. Bright field images were taken at 10× and 20× magnifications.

### 5.4.14. TRACP and ALP Double Staining of Paraffin Embedded Bone Sections

TRACP and ALP double staining was performed on 4 μm bone sections using TRACP & TRAP double stain kit (Cat #mK300, Takara) following the manufacturer's guidelines. Briefly, 500 ul of acid phosphatase (ACP) substrate was added to the bone tissue and incubated for 45 minutes at 37° C. followed by washing in ddH₂O. 500 ul alkaline phosphatase (ALP) substrate was then added to the tissue sections and incubated at 37° C. for 45 minutes. The washed bone tissues were counterstained with the nuclear staining reagent for 5 minutes at room temperature. The bone tissues were washed, air-dried, mounted with cover slips using mounting medium and viewed under Eclipse 80i compound fluorescent microscope. The number of TRAP⁺ and ALP⁺ cells at the bone defect sites and growth plates were quantified using Image J Software.

### 5.4.15. Transmission Electron Microscopy (TEM)

The harvested bone tissues from the sham control and HKUOT-S2 treatment groups were decalcified. 2 mm of the bone tissue that included the bone defect sites were processed for TEM analysis at the Electron Microscopy Unit, HKU. The Philips CM100 TEM was used to acquire the detailed images of the bone tissue sections at different magnifications (1200-5200X) for qualitative cellular and intracellular ultrastructural analyses.

### 5.4.16. Enzyme-Linked Immunosorbent Assay (ELISA)

The HKUOT-S2-induced ALP and OCN levels during the bone defect repairs were evaluated by mouse BALP (Cat #CSB-E11914m, CUSABIO) and mouse OC/GPG (Cat #NBP2-68151, NOVUS BIOLOGICAL) ELISA kits according to the manufacturers' instructions. In short, duplicates of 100 μl of freshly prepared standards, sera and bone lysate samples from the sham control and HKUOT-S2 treatments groups were added to the micro-ELISA plates and incubated at 37° C. for 90 or 120 minutes. The standards and sample solutions were replaced with freshly prepared 100 μl biotin-antibody solution and incubated at 37° C. for 60 minutes with gentle shaking. The micro-ELISA plates were washed 4 times with 200 μl washing buffer. 100 μl of freshly prepared 1×HRP conjugated working solution or 1×HRP-avidin was added to each well and incubated for 37° C. for 30 or 60 minutes followed by 4 times washing. The sealed micro-ELISA plates containing 90 μl of substrate reagent or TMB substrate were incubated for 37° C. for 15 minutes in the dark. 50 μl of stop solution was then added to the micro-ELISA plates and gently tapped to ensure thorough mixing. The OD values of each well were spectrophotometrically determined at 450 nm. The standard curve linear equations were used to determine BALP and OCN levels in the sera and bone lysates from the sham control and HKUOT-S2 treatment groups. The total proteins measured were used to normalize the BALP and OCN levels in the bone lysate samples.

5.4.17. rRNA-Depleted RNA-Sequencing

The harvested femurs from the sham and HKUOT-S2 treatment groups were wrapped in aluminum foil and ground in liquid nitrogen using mortar and pestle to prevent RNA degradation. High quality total RNAs were double extracted from the bone the tissues using RNAiso plus reagent (Cat #9109, Takara) and PureLink™ RNA Mini Kit (Cat #12183018A, Thermo Scientific). The resultant RNA solutions also had OD260/OD280 between 1.98-2.00 and RNA integrity number (RIN)>8. The RNA samples for the sham control (n=3), 1.09 mg kg⁻¹ (n=3), 2.18 mg kg⁻¹ (n=3), and 4.36 mg kg⁻¹ (n=3) HKUOT-S2 treatment groups were processed for RNA sequencing (RNA-seq). The library preparation, Illumina sequencing (Pair-End sequencing of 151 bp) and transcriptome bioinformatics were performed at LKS Faculty of Medicine, Centre for PanorOmic Sciences (CPOS), Genomics Core, HKU. Prior to the RNA-seq process, the bulk rRNA in the 0.5 ug RNA samples were depleted using the QIAseq® FastSelect™ Multi-RNA Removal Kit (Human Complete rRNA & Globin mRNA, Cat #THS-201Z-24), to ensure that the RNA-seq quality captured the most informative portions of the transcriptomic data. ^{[74]}Cytoplasmic and mitochondrial rRNAs were depleted using the QIAseq® FastSelect™ reagents during the next-generation sequencing (NGS) library preparation. The cDNA libraries were prepared using the KAPA mRNA HyperPrep Kit (KR1352-v.116). The double-stranded (ds) cDNA underwent, 3′ adenylation and indexed adaptor ligation using xGen® Dual Index UMI Adapters. The adaptor-ligated libraries were enriched by 12 cycles of polymerase chain reaction (PCR), denatured, and diluted to optimal concentration. Pair-End 151 bp sequencing was performed using Illumina NovaSeq 6000. The RNA-seq reads were assigned to each experimental sample using the Illumina (bcl2fastq) software. Each sample had an average 12.1 Gb throughputs and 144.6 Gb total throughputs. In terms of sequence quality, an average of 92% of the bases achieved a quality score of Q30 where Q30 denotes the accuracy of a base call to be 99.9%. Transcriptome pair-wise bioinformatics analysis was used to generate HKUOT-S2-induced differentially expressed genes and transcripts in the defective bones. The HKUOT-S2-induced gene enriched KEGG pathways and GO terms were evaluated using the Partek genomic suite (PGS) software from CPOS, HKU. Transcript per million (TPM) values from RNA-seq were utilized for further analyses. The unsupervised hierarchical clustering was plotted with gplots using z-scores calculated for each gene across samples. ^{[75]}Most variable 1000 genes were included for the analysis.

### 5.4.18. Validation of the HKUOT-S2-Induced Differentially Expressed Genes in the Repaired Bones

The RNAs from the bones of the sham control and HKUOT-S2 treatment groups were subjected to qPCR analysis to validate the HKUOT-S2-induced differentially expressed genes related to BMP, TGF-β, AMPK and mTOR signaling pathways that promoted bone defect repairs in vivo. The HKUOT-S2-induced AMPK and mTOR related genes (Prkaa1, Prkaa2, Prkab1, Prkab2, Prkag1, Prkag2, Prkag3 and Mtor), BMP and TGF-β related genes (Bmp2, Bmp7 and Bmpr2, Tgfβr2) were validated by qPCR.

### 5.5. In Vitro Studies

### 5.5.1. In Vitro Cell Culture

The hMSCs and human monocytes, U-937 cell lines were cultured and maintained in T75 flasks containing 10 ml DMEM low glucose medium and cell culture dishes containing 10 ml RMPI medium 1640 (1×) respectively. Both cell culture media were supplemented with 10% heat activated FBS,100 Uml⁻¹ P/S. The RAW264.7 and MC3T3-1E cell lines were cultured and maintained in cell culture dishes and T75 flasks respectively, containing 10 ml DMEM high glucose medium supplemented with 3.72 mg/ml NaHCO₃, 5.685 mgml⁻¹, HEPES, 10% FBS, 2 mM L-glutamine), 0.25 μg ml⁻¹ amphotericin B, 100 Uml⁻¹ P/S. The hMSCs and MC3T3-1E cell lines were cultured and maintained for 4-5 days with the cell culture medium replacement every 3 days. The cells were passaged at 80% confluence by washing twice with warmed 1×PBS, adding 1 ml of 0.25% trypsin-EDTA to each flask and incubated at 37° C. for 3 minutes to detach the cells, addition of 5 ml normal cell culture medium to quench the cell trypsinization action, centrifugation at 1000 rpm for 5 minutes to collect cell pellets, resuspension of cell pellets with 1 ml culture medium and finally, adding 100 ul of the cell suspension to 10 ml culture medium in the new T75 flask. The U-937 and RAW264.7 cells were passed every 3 days by gently wash the cells twice with 5 ml of warm 1×PBS, dislodging the loosing attached cells with 3 ml of freshly warmed cell culture medium by pipetting and adding 100 ul of the cell suspension to 10 ml culture medium in the new cell culture dish. All the cells were kept in the humidified incubators at 37° C. and 5% CO₂.

5.5.2. Effects of HKUOT-S2 Treatments on hMSCs and RAW264.7 Cells Viability

The hMSCs, and RAW264.7 cells were seeded in triplicates at 1.5×10³ cell/well and 2×10⁴ cells/well of 96-well plates respectively with or without HKUOT-S2. The cells were incubated under cell culture conditions for day 1, 2 and 3 time points. At each time point, the cells were washed with FBS-free medium. 50 ul of the FBS-free medium with final concentration of 0.45 mgml⁻¹ MTT was then added per well and incubated in the dark for 4 hrs at 37° C. and 5% CO2 after which the medium was carefully discarded. 100 ul DMSO was added to each well and incubated for 15 minutes to dissolve the formazan crystals. The optical densities of the triplicate wells were measured at the absorbance (OD) 570 nm (OD570) and 640 nm (OD640) references. The % cell viability was calculated as:

$$Cell viability ( \% ) = \frac{( OD 570 - OD 640 ) of treatment}{( OD 570 - OD 640 ) of control )} * 100$$

5.5.3. Effects of HKUOT-S2 on hMSCs to Osteoblast Differentiation

The hMSCs and MC3T3-1E cell lines were seeded at 2.5×10⁴ cells/well and 2.5×10³ cells/well of 6-well plate respectively, containing 2 ml of normal cell culture media. The cell culture media were then replaced with osteogenic media containing 100 nM Dex, 10 mM β-Gly and 0.28 mM Asc with or without the HKUOT-S2 protein. The cells were maintained in the osteogenic media for 14 days with media replacement every 3 days. The nondifferentiated cells maintained in the normal cell culture media were used as controls. The cell pellets were then collected and processed for osteogenic gene expression analysis by qPCR at the experimental endpoints.

### 5.5.4. Effects of HKUOT-S2 on Macrophage Polarizations

RAW264.7 cells were seeded at 2×10⁵ cells/well of 6-well plates and cultured for 24 hrs. The cells were then polarized separately into M1 and M2 macrophages using 100 ng ml⁻¹ LPS and 20 ng ml⁻¹ mouse TL-4 respectively for 24 hrs with or without 0.01-1.0 μg ml⁻¹ HKUOT-S2 protein. The U937 cells were seeded at 2×10⁴ cells/well of 6-well plates and cultured for 24 hrs. The U937 cell culture medium was supplemented with 20 ng ml⁻¹ final concentration of PMA and incubated for 48 hrs to activate the suspended pro-monocytes to attach to the bottom of the wells and differentiate into mature M0 macrophages. The attached cells were washed twice with 1×PBS and subjected to M1 and M2 macrophage polarization using 100 ng ml⁻¹ LPS and 20 ng ml⁻¹ human TL-4 respectively, for 24 hrs, with or without 0.01-1.0 μg ml⁻¹ HKUOT-S2 protein. The non-polarized cells were used as controls. The RAW264.7 and U937-derived M1 and M2 macrophages were scraped, and the collected pellets were processed for qPCR, flow cytometry and western blot analyses. The condition media (CM) from the U937-derived M1 and M2 macrophages were collected for hMSCs-osteoblast differentiation and cytokine array analyses.

5.5.5. Effects of HKUOT-S2 and HKUOT-P-Induced M1 and M2 Macrophage CM on hMSCs-Derived Osteoblast Biomineralization

The hMSCs seeded at 1×10⁴ cells/well into 24-well plates were cultured for 24 hrs. The cell culture medium was replaced with medium containing osteogenic medium and the U937 cell-derived macrophage CM in 1:1 ratio with continuous presence or absence of HKUOT-S2 and HKUOT-P proteins. The cells were maintained in the osteogenic-macrophage CM media for 10 days with media replacement every 3 days. At the experimental endpoint, the cells were gently washed 4× with warmed 1×PBS and fixed with 4% paraformaldehyde (4% PFA) for 15 minutes at room temperature. The fixed cells were gently washed 4× with ddH₂O followed by staining with 0.5 ml of 40 mM Alizarin red S (ARS) in the dark at room temperature for 45 minutes. The stained cells were gently washed 6× with ddH₂O. The plates were air-dried overnight in the hood after which 0.2 ml of 10% acetic acid was added to each well with gentle shaking for 45 minutes. Th cells scraped in the acetic acid were transferred into 1.5 ml Eppendorf tubes, and vortexed for 30 seconds. The sealed tubes were put in the heat block for 10 minutes at 85° C. The tubes were then cooled on ice for 5 minutes followed by 15 minutes centrifugation at 20000 g. 200 ul of the supernatants were transferred into clean 0.5 ml Eppendorf tubes and 75 ul of 10% NH₄OH was added to each tube to quench the action of the acid. The ARS standards were prepared by serial dilutions as 40 mM, 20 mM, 10 mM, 5 mM, 0.25 mM, 0.125 mM, 0.0625 mM, and 0 mM. 50 ul 0f the standards and the samples in triplicates were transferred into the 96-well plates and the absorbances read at 405 nM using the microplate reader. The generated linear equation from the standard curve was used to determine the amount of biomineralization in each treatment group.

5.5.6. HKUOT-S2 Treatment Suppressed XL388-Induced mTOR Inhibition to Promote Osteoblast Differentiation

hMSCs were differentiated into osteoblasts with/without 125-500 XL388 or 0.1 μg ml⁻¹ HKUOT-S2 treatments for 7 days with medium replenishments every 3 days. The collected cell pellets were processed for qPCR and western blot analyses to establish the mechanism by which HKUOT-S2 promotes osteogenesis and new bone formation. The lists of all the genes and corresponding primer pairs as well as primary and secondary antibodies used for qPCR and western blot analyses are listed in Tables 6, 7 and 8.

TABLE 6
Mouse genes and primer pairs for qPCR

| Gene | Primer (mouse) | |
|---|---|---|
| Alp | Forward: | 5′-CCAGCAGGTTTCTCTCTTGG-3′ (SEQ ID |
| | | NO: 1) |
| | Reverse: | 5′-GGGATGGAGGAGAGAAGGTC-3′ (SEQ ID |
| | | NO: 2) |
| Col | Forward: | 5′-GAGCGGAGAGTACTGGATCG-3′ (SEQ ID |
| | | NO: 3) |
| | Reverse: | 5′-GTTCGGGCTGATGTACCAGT-3′ (SEQ ID |
| | | NO: 4) |
| Opn | Forward: | 5′-TCTGATGAGACCGTCACTGC-3′ (SEQ ID |
| | | NO: 5) |
| | Reverse: | 5′-AGGTCCTCATCTGTGGCATC-3′ (SEQ ID |
| | | NO: 6) |
| Runx2 | Forward: | 5′-CCCAGCCACCTTTACCTACA-3′ (SEQ ID |
| | | NO: 7) |
| | Reverse: | 5′-TATGGAGTGCTGCTGGTCTG-3′ (SEQ ID |
| | | NO: 8) |
| Arg-1 | Forward: | 5′-CTCCAAGCCAAAGTCCTTAGAG-3′ (SEQ |
| | | ID NO: 9) |
| | Reverse: | 5′-AGGAGCTGTCATTAGGGACATC-3′ (SEQ |
| | | ID NO: 10) |
| Mgl-1 | Forward: | 5′-TGAGAAAGGCTTTAAGAACTGGG-3′ |
| | | (SEQ ID NO: 11) |
| | Reverse: | 5′-GACCACCTGTAGTGATGTGGG-3′ (SEQ |
| | | ID NO: 12) |
| Cd206 | Forward: | 5′-TTCCATCGAGACTGCTGCT-3′ (SEQ ID |
| | | NO: 13) |
| | Reverse: | 5′-CCAGAGGGATCGCCTGTTT-3′ (SEQ ID |
| | | NO: 14) |
| Ym1 | Forward: | 5′-CATGAGCAAGACTTGCGTGAC-3′ (SEQ |
| | | ID NO: 15) |
| | Reverse: | 5′-GGTCCAAACTTCCATCCTCCA-3′ (SEQ ID |
| | | NO: 16) |
| Socs3 | Forward: | 5′-GATTTCGCTTCGGGACTAG-3′ (SEQ ID |
| | | NO: 17) |
| | Reverse: | 5′-CGGCGGCGGGAAACTTGCTG-3′ (SEQ ID |
| | | NO: 18) |
| Tnfα | Forward: | 5′-TCTCAGCCTCTTCTCATTCCTGC-3′ (SEQ |
| | | ID NO: 19) |
| | Reverse: | 5′-AGAACTGATGAGAGGGAGGCCAT-3′ |
| | | (SEQ ID NO: 20) |
| iNOS | Forward: | 5′-AATCTTGGAGCGAGTTGTGG-3′ (SEQ ID |
| | | NO: 21) |
| | Reverse: | 5′-CAGGAAGTAGGTGAGGGCTTG-3′ (SEQ |
| | | ID NO: 22) |
| IL-6 | Forward: | 5′-ACAAAGCCAGAGTCCTTCAGAGAG-3′ |
| | | (SEQ ID NO: 23) |
| | Reverse: | 5′-TTGGATGGTCTTGGTCCTTAGCCA-3′ |
| | | (SEQ ID NO: 24) |
| IL-1β | Forward: | 5′-AGAGCTTCAGGCAGGCAGTA-3′ (SEQ ID |
| | | NO: 25) |
| | Reverse: | 5′-AGGTGCTCATGTCCTCATCC-3′ (SEQ ID |
| | | NO: 26) |
| Mcp-1 | Forward: | 5′-CCAGCAAGATGATCCCAATG-3′ (SEQ ID |
| | | NO: 27) |
| | Reverse: | 5′-TTCTTGGGGTCAGCACAGAC-3′ (SEQ ID |
| | | NO: 28) |
| Prkaa1 | Forward: | 5′-GGTGTACGGAAGGCAAAATGGC-3′ (SEQ |
| | | ID NO: 29) |
| | Reverse: | 5′-CAGGATTCTTCCTTCGTACACGC-3′ (SEQ |
| | | ID NO: 30) |
| Prkaa2 | Forward: | 5′-CTGAAGCCAGAGAATGTGCTGC-3′ (SEQ |
| | | ID NO: 31) |
| | Reverse: | 5′-GAGATGACCTCAGGTGCTGCAT-3′ (SEQ |
| | | ID NO: 32) |
| Prkab1 | Forward: | 5′-CCAAAAGTGCTCCGATGTGTCTG-3′ (SEQ |
| | | ID NO: 33) |
| | Reverse: | 5′-GGGCTTTGAACCTCTCTTCTGG-3′ (SEQ |
| | | ID NO: 34) |
| Prkab2 | Forward: | 5′-GACTTCGTTGCCATCCTGGATC-3′ (SEQ |
| | | ID NO: 35) |
| | Reverse: | 5′-CCAAGCTGACTGGTAACCACAG-3′ (SEQ |
| | | ID NO: 36) |
| Prkag1 | Forward: | 5′-TCTCCGCCTTACCTGTAGTGGA-3′ (SEQ |
| | | ID NO: 37) |
| | Reverse: | 5′-GCAGGGCTTTTGTCACAGACAC-3′ (SEQ |
| | | ID NO: 38) |
| Prkag2 | Forward: | 5′-CTCCTCATCCAAAGAGTCTTCGC-3′ (SEQ |
| | | ID NO: 39) |
| | Reverse: | 5′-TGGGTGTTGACGGAGAAGAGGA-3′ (SEQ |
| | | ID NO: 40) |
| Prkag3 | Forward: | 5′-AAGCGGCTACTCAAGTTCCTGC-3′ (SEQ |
| | | ID NO: 41) |
| | Reverse: | 5′-CCAGAACTACAGCCAAATCTCGG-3′ |
| | | (SEQ ID NO: 42) |
| Gapdh | Forward: | 5′-CATCACTGCCACCCAGAAGACTG-3′ |
| | | (SEQ ID NO: 43) |
| | Reverse: | 5′-ATGCCAGTGAGCTTCCCGTTCAG-3′ (SEQ |
| | | ID NO: 44) |
| Bglap1 | Forward: | 5′-GCAATAAGGTAGTGAACAGACTCC-3′ |
| | | (SEQ ID NO: 45) |
| | Reverse: | 5′-CCATAGATGCGTTTGTAGGCGG-3′ (SEQ |
| | | ID NO: 46) |
| Bglap2 | Forward: | 5′-GCAATAAGGTAGTGAACAGACTCC-3′ |
| | | (SEQ ID NO: 47) |
| | Reverse: | 5′- GCGTTTGTAGGCGGTCTTCAAG-3′ (SEQ |
| | | ID NO: 48) |
| Got1 | Forward: | 5′-TGCTACTGGGATGCGGAGAAGA-3′ (SEQ |
| (Ast) | | ID NO: 49) |
| | Reverse: | 5′-TGCATGACAGCAGCGATCTGCT-3′ (SEQ |
| | | ID NO: 50) |
| Gpt1 | Forward: | 5′-CCACTCAGTCTCTAAGGGCTAC-3′ (SEQ |
| (Alt) | | ID NO: 51) |
| | Reverse: | 5′-ACACAACCGCACGCTCATCAGT-3′ (SEQ |
| | | ID NO: 52) |
| Ckb | Forward: | 5′-GCTCATTGACGACCACTTCCTC-3′ (SEQ |
| (Creatine) | | ID NO: 53) |
| | Reverse: | 5′-CCTCCTCGTTAATCCACACCAG-3′ (SEQ |
| | | ID NO: 54) |
| mTOR | Forward: | 5′-AGAAGGGTCTCCAAGGACGACT-3′ (SEQ |
| | | ID NO: 55) |
| | Reverse: | 5′-GCAGGACACAAAGGCAGCATTG-3′ (SEQ |
| | | ID NO: 56) |
| Bmp2 | Forward: | 5′-AACACCGTGCGCAGCTTCCATC-3′ (SEQ |
| | | ID NO: 57) |
| | Reverse: | 5′-CGGAAGATCTGGAGTTCTGCAG-3′ (SEQ |
| | | ID NO: 58) |
| Bmpr2 | Forward: | 5′-AGAGACCCAAGTTCCCAGAAGC-3′ (SEQ |
| | | ID NO: 59) |
| | Reverse: | 5′-TCTCCTCAGCACACTGTGCAGT-3′ (SEQ |
| | | ID NO: 60) |
| Bmp7 | Forward: | 5′-GGAGCGATTTGACAACGAGACC-3′ (SEQ |
| | | ID NO: 61) |
| | Reverse: | 5′-AGTGGTTGCTGGTGGCTGTGAT-3′ (SEQ |
| | | ID NO: 62) |
| Tgfbr2 | Forward: | 5′-CCTACTCTGTCTGTGGATGACC-3′ (SEQ |
| | | ID NO: 63) |
| | Reverse: | 5′-GACATCCGTCGTCTTGAACGAC-3′ (SEQ |
| | | ID NO: 64) |

TABLE 7
Human genes and primer pairs for qPCR

| Gene | Primers (human) | |
|---|---|---|
| ALP | Forward: | 5′-ATGGGATGGGTGTCTCCACA-3′ (SEQ ID NO: |
| | | 65) |
| | Reverse: | 5′-CCACGAAGGGGAACTTGTC-3′ (SEQ ID NO: |
| | | 66) |
| COL1A | Forward: | 5′-ATGACTATGAGTGGGAAGCA-3′ (SEQ ID NO: |
| | Reverse: | 67) |
| | | 5′-TGGGTCCCTCTGTTACACTTT-3′ (SEQ ID NO: |
| | | 68) |
| OPN | Forward: | 5′-CTCAGGCCAGTTGCAGCC-3′ |
| | | (SEQ ID NO: 69) |
| | Reverse: | 5′-CAAAAGCAAATCACTGCAATTCTC-3′ (SEQ ID |
| | | NO: 70) |
| RUNX2 | Forward: | 5′-CCTGAACTCTGCACCAAGTC-3′ (SEQ ID NO: |
| | | 71) |
| | Reverse: | 5′-GAGGTGGCAGTGTCATCATC-3′ (SEQ ID NO: |
| | | 72) |
| GAPDH | Forward: | 5′-GGCATCCACTGTGGTCATGAG-3′ (SEQ ID NO: |
| | | 73) |
| | Reverse: | 5′-TGCACCACCAACTGCTTAGC-3′ (SEQ ID NO: |
| | | 74) |
| 4EBP1 | Forward: | F: 5′-CACCAGCCCTTCCAGTGATGAG-3′ (SEQ ID |
| | | NO: 75) |
| | Reverse: | R: 5′-CCTTGGTAGTGCTCCACACGAT-3′ (SEQ ID |
| | | NO: 76) |
| AKT1 | Forward: | F: 5′-TGGACTACCTGCACTCGGAGAA-3′ (SEQ ID |
| | | NO: 77) |
| | Reverse: | R: 5′-GTGCGGCAAAAGGTCTTCATGG-3′ (SEQ |
| | | ID NO: 78) |
| S6K1 | Forward: | F: 5′-TATTGGCAGCCCACGAACACCT-3′ (SEQ ID |
| | | NO: 79) |
| | Reverse: | R: 5′-GTCACATCCATCTGCTCTATGCC-3′ (SEQ |
| | | ID NO: 80) |

TABLE 8
Primary and Secondary antibodies for western blot

| | | |
|---|---|---|
| Name | Catalogue number | Company |

Primary Antibodies

| | | |
|---|---|---|
| Arginase-1 (D4E3M ™) | 93668S | Cell Signaling |
| XP ® Rabbit mAb | | Technology |
| Phospho-4E-BP1 | 2855S | Cell Signaling |
| (Thr37/46) (236B4) | | Technology |
| Rabbit mAb | | |
| 4E BP1 | 9452S | Cell Signaling |
| | | Technology |
| Phospho-mTOR (Ser2448) | 2971S | Cell Signaling |
| | | Technology |
| mTOR (7C10) | 2983S | Cell Signaling |
| | | Technology |
| Phospho-p70 S6 | 9205S | Cell Signaling |
| Kinase | | Technology |
| (Thr389) | | |
| Phospho-Akt1 (Ser473) | 9018S | Cell Signaling |
| (D7F10) XP ® | | Technology |
| Rabbit mAb | | |
| (Akt1 Specific) | | |
| Beta-Actin Mouse | 66009-1-Ig | Proteintech |
| Monoclonal | | |

Secondary Antibodies

| | | |
|---|---|---|
| ECL ™ Anti-rabbit IgG- | NA934 | Amersham |
| HRP | | |
| ECL Mouse IgG, HRP- | NA931-1ML | Cyvita |
| linked whole Ab | | |

### 5.6 Experimental Results Analyses and Evaluations

5.6.1. Quantitative Real-Time PCR (qPCR) Analysis

High Quality Total RNAs, with OD260/OD280 Between 1.98-2.00, were extracted from the in vitro experimented cell lines and the tissues harvested from the in vivo experiments using RNAiso plus reagent (Cat #9109) according to the manufacturer's protocol. The quality and quantity of the extracted RNAs were determined using the NanoDrop One (C) UV-VIS micro-volume spectrophotometer (Thermo Scientific). Equal amount of RNAs across the experimental groups were reverse transcribed into complementary DNA (cDNA) using the PrimeScript™ RT reagent Kit (cat #RR037A, Takara) and Peltier Thermal Cycler (Cat #PTC-100, MJ Research) according to the manufacturers' instructions. Specific primer pairs, equal amount of cDNA for the various experimental groups and the PowerUp™ SYBR™ Green Master Mix (Cat #A25742, Thermo Scientific) were used to prepare the qPCR reaction mix. 10 μl of the qPCR reaction mix of the control and HKUOT-S2 treatment groups, loaded into 96-well plate (0.2 ml) were used evaluate the HKUOT-S2-induced gene expressions in the cell lines from the in vitro and the harvested tissues from the in vivo experiments using the QuantStudio 5 real-time PCR system (Thermo Scientific). The HKUOT-S2-induced differentially gene expression fold changes were calculated by 2^{-ΔΔCt} method after housekeeping gene normalization. The lists of all the genes and corresponding primer pairs used for qPCR are listed in Table 3.

TABLE 3
De novo peptide sequence of HKUOT-S2 protein

| | | | | | | Blastp |
|---|---|---|---|---|---|---|
| | | | | | | match |
| | | | | | Post- | against |
| | De novo | | | | translational | NCBInr |
| Peptide | score | charge | Mass | | modification | Dioscorea |
| sequence | (%) | (z) | (m) | m/z | (PTM) | Genus protein |
| KTVSLPR | 98 | 2 | 841.50 | 421.75 | Acetylation (N- | No |
| (SEQ ID NO: | | | | | term) | significant |
| 81) | | | | | | match |
| KGNLLECDG | 96 | 2 | 2038.88 | 1020.45 | Acetylation (N- | less |
| G- | | | | | term); | |
| NTAQMMAR | | | | | Carbamidomethy | |
| (SEQ ID NO: | | | | | lation; Oxidation | significant |
| 82) | | | | | (M) | match |
| TKSSLPGQT | 96 | 2 | 1087.58 | 544.80 | Acetylation (N- | No |
| K (SEQ ID | | | | | term) | significant |
| NO: 83) | | | | | | match |
| KEVSLPR | 96 | 2 | 869.49 | 435.75 | Acetylation (N- | No |
| (SEQ ID NO: | | | | | term) | significant |
| 84) | | | | | | match |

### 5.6.2. Flow Cytometry Analysis of HKUOT-S2 Treated Primary M0-Derived Macrophages

Primary bone marrow macrophages were harvested from the 8 weeks old mice according to the published protocol. ^{[76]}Briefly, the mice were euthanized with pentobarbital (100 mg kg⁻¹). The femurs were harvested into cell culture dish under aseptic conditions. In the sterile cell culture hood, 10 ml of 70% ethanol was added to the femurs for 1 minute. The femurs, washed 3× with warmed 1×PBS, were transferred into another 100 mm cell culture dish containing 10 ml mouse macrophage (RAW264.7 cells) culture medium. A pair of scissors was used to cut both ends of the femurs. Sterile forceps were used to hold the femurs vertically. 26 G×½″ needle, attached to 10 ml syringe, filled with cell culture medium, was used to flash out the bone marrow cells several times into the 50 ml tubes. The cell suspensions were passed through 70 μm cell strainer into another 50 ml tubes followed by centrifugation at 1000 rpm for 5 minutes. The cell pellets were resuspended in 100 mm cell culture dish containing 10 ml cell culture medium supplemented with 10 ng ml⁻¹ murine recombinant M-CSF. The cells were incubated under cell culture conditions for one week with medium replacement every 3 days. On day 7, the cells were passaged with cell culture medium without M-CSF supplementation. The primary M0 macrophages were seeded at 2×10⁵ cells/well of 6-well plates and cultured under suitable cell culture conditions for 24 h. The cells were then polarized separately into M1 and M2 macrophages using 100 ng ml⁻¹ LPS and 20 ng ml⁻¹ mouse TL-4 respectively for 24 h with or without HKUOT-S2 or HKUOT-P1 protein treatments. 0.5 ml of singleton cells suspension with concentration of 2.0×10⁷ cells/ml were processed and co-stained with fluorescent labelled CD206, and MGL-1 antibodies for flow cytometry analysis using Agilent NovoCyte Quanteon analyzer.

### 5.6.3. Cytokine Array Analysis of HKUOT-S2 Polarized M1 and M2 Macrophage CM

The cytokine array analysis of the HKUOT-S2 induced M1 and M2 macrophage CM was performed using the Mouse XL Cytokine Array Kit (Cat #ARY028, R&D Systems) according to the manufacturer's protocol. Briefly, the 4 membranes, placed in the wells 1-4 of the 4-Well Multi-dish containing 2 ml blocking buffer were incubated for 1 hr on a shaker. The blocking buffers were replaced with 1.5 ml of each sample solution such that wells 1-4 contained M2 (M2 macrophage positive control), M1(M1 macrophage positive control), M1+HKUOT-S2 and M1+HKUOT-S2 CM respectively. The membranes were incubated on a shaker at 4° C. overnight and washed with 1× Wash Buffer several times. The membranes in 1.5 mL of 1× Array Buffer 4/6 containing 30 μl Detection Antibody Cocktail were incubated at room temperature with shaking followed by washing with the 1× Wash Buffer. Each membrane was incubated in 2.0 mL of 1× Streptavidin-HRP at room temperature for 30 minutes, washed, and treated with 1 ml Chemi Reagent Mix for 60 seconds. The detected cytokine array intensities were visualized using GE Amersham Imager AI680 and quantified by the ImageJ software.

### 5.6.4. Western Blot Analysis

RAW264.7 cells were seeded at 2×10⁵ cells/well and cultured for 24 h. The cells were polarized into M1 and M2 macrophages with or without 0.1 μg ml⁻¹ HKUOT-S2 or 5 μg ml⁻¹ HKUOT-P1. The collected cell pellets were lysed with 60 μl RIPA buffer (Cat #89900, Thermo Scientific) supplemented with 25× cOmplete™, EDTA-free Protease Inhibitor Cocktail (Cat #1187358001, Roche Diagnostics). The total protein concentrations were measured spectrophotometrically using Pierce Coomassie Plus (Bradford) Protein Assay kit (Cat #23236, thermo scientific) and Pierce™ Bovine Serum Albumin Standard (Cat #23209) according to the manufacturer's instructions. 30 μg of the protein lysate (12.5 ul reaction mix) and 10 μl PageRuler™ Plus Prestained Protein Ladder (Cat #26619, Thermo Scientific), separated through 8% SDS-PAGE in running buffer, were transferred to PVDF membranes for 2 h in cold transfer buffer. The PVDF membranes were blocked with 10 ml of PBST (0.5 ml Tween 20 in 1 L PBS) containing 5% BSA (Cat #A-420-250, GoldBio) for 30 minutes. The PVDF membranes were then incubated in 10 ml of blocking buffer (PBST+5% BSA) containing 10 ul of Arginase-1 (D4E3M™) XP® Rabbit mAb (1:1000 dilution) (Cat #93668, Cell Signaling Technology) with gentle shaking at 4° C. overnight. The washed membranes were incubated with ECL™ Anti-rabbit IgG-HRP secondary antibody (Cat #NA934, Amersham) on the shaker for 2 h at room temperature. The membranes, washed with 1×PBST, were incubated with Pierce™ ECL Western Blotting Substrate (Cat #32209, Thermo Scientific) for 1 minutes. The ARG-1 protein band intensities were visualized using GE Amersham Imager AI680 and quantified using the ImageJ software. The lists of all the primary and secondary antibodies used for western blot are listed in Table 4.

TABLE 4
Number of differentially expressed genes and transcripts

| | Number of | Number of |
|---|---|---|
| | differentially | differentially |
| | expressed | expressed |
| Sample pair | genes | transcripts |
| CLT vs 1.09 mg kg⁻¹ | 6,038 | 9,477 |
| CLT vs 2.18 mg kg⁻¹ | 6,158 | 9,974 |
| CLT vs 4.36 mg kg⁻¹ | 1,064 | 2,871 |

All patents, patent applications, provisional applications, and publications referred to or cited herein are incorporated by reference in their entirety, including all figures and tables, to the extent they are not inconsistent with the explicit teachings of this specification.

The following are examples that illustrate procedures for practicing the invention. These examples should not be construed as limiting. All percentages are by weight and all solvent mixture proportions are by volume unless otherwise noted.

### Example 1—Novel HKOUT-S2 Protein was Isolated from Dioscorea opposita Thunb

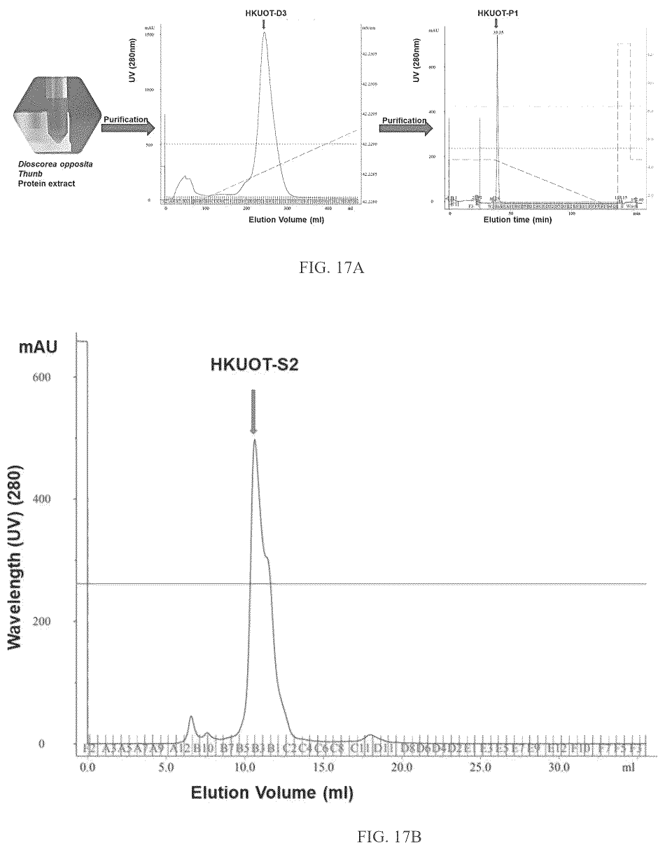

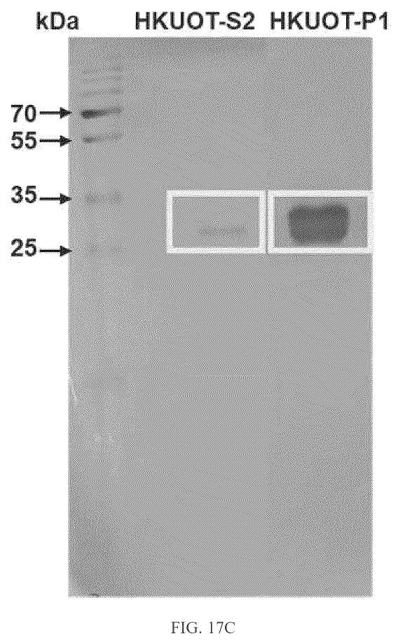

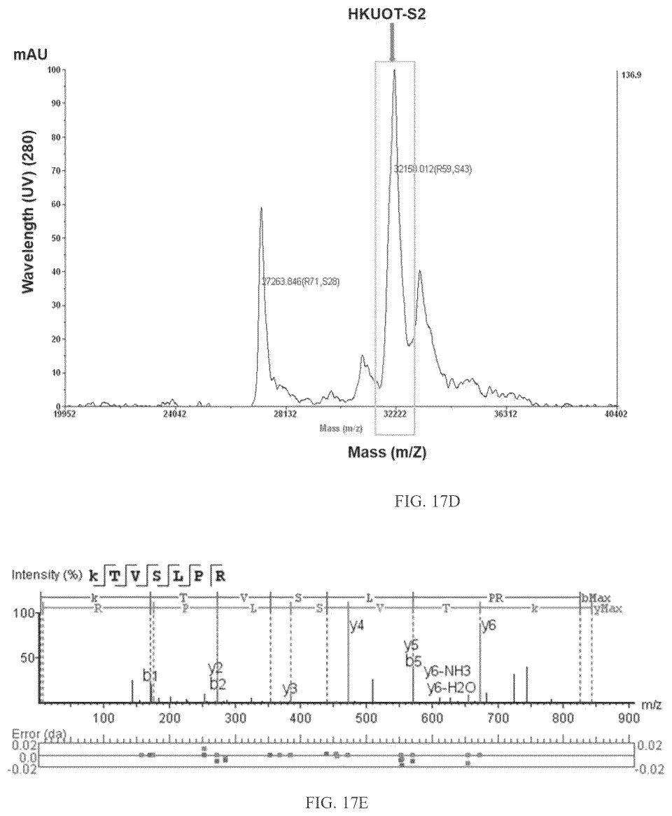

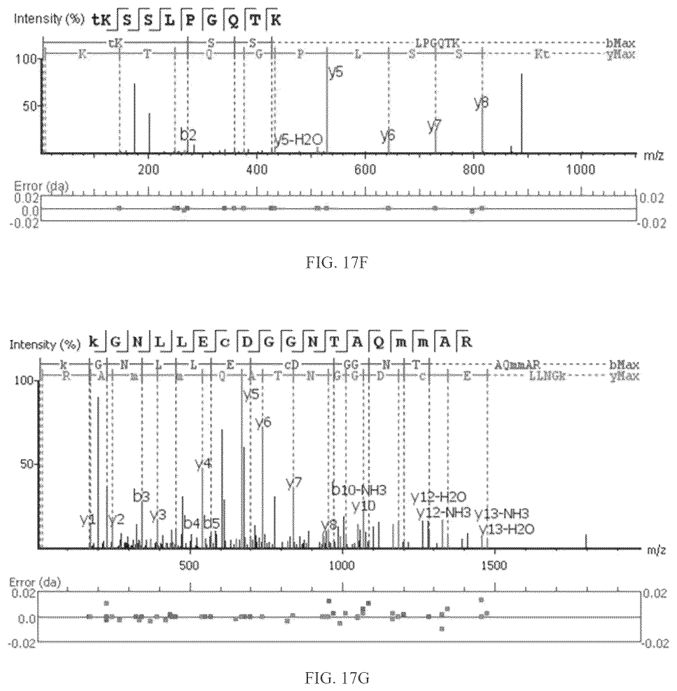

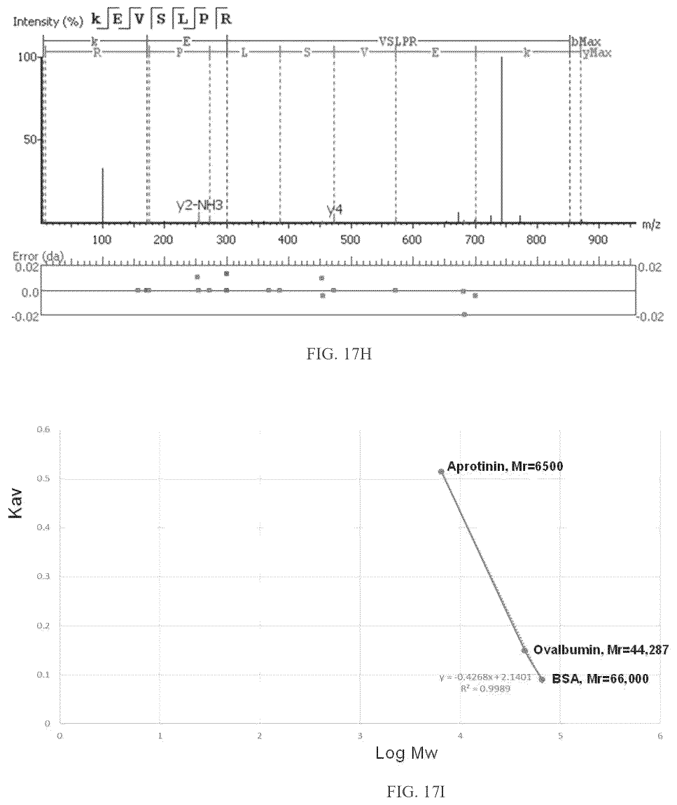

To isolate osteogenic novel HKUOT-S2 protein from Dioscorea spp, crude proteins were extracted from the tubers of the Dioscorea opposita Thunb by high-speed centrifugation (FIG. 17A). The novel HKUOT-S2 protein was isolated from the crude protein extract using ion exchange, hydrophobic interaction, and high-resolution size-exclusion chromatographic techniques to successively and sequentially isolate the osteogenic fractions D3 (HKUOT-D3), P1 (HKUOT-P1) and novel S2 (HKUOT-S2) protein (FIGS. 17A-17B) respectively.

### Example 2—Phytochemical Screening of Crude Protein Extracts and Fractions

Dioscorea spp. are not only carbohydrate rich tuber plants, but also contain other essential components such as water, inulin, tannins, organic acids, phenolics, proteins, and antioxidants. ^{[39, 40]}The Dioscorea opposita Thunb crude protein extract and its successive purified derivatives were subjected to qualitative phytochemical screening to determine the presence or absence of carbohydrates, saponin, phytosterols, phenols, flavonoids, amino acids and proteins. ^{[41]}As expected, the result showed that the crude protein extracts, HKUOT-D3 and HKUOT-P1, do not contain carbohydrates (Table 1). The crude protein extract however, contained saponins, phytosterols-triterpenes and flavonoids which were absent in the HKUOT-D3 and HKUOT-P1. Both HKUOT-D3 and HKUOT-P1 contained amino acids, and proteins (Table 1). It could therefore be deduced from the phytochemical results that HKUOT-S2 derived from HKUOT-P1 was a protein molecule.

TABLE 1
Phytochemical analysis of yam protein extracts and isolates

| | | | Phytosterols- | | | | |
|---|---|---|---|---|---|---|---|
| | | | Triterpenes | | | | |
| | | | (Shalkowski's | | | | |
| | | | test) | | Flavonoids | | |
| | Carbohydrate | | Sample + | Phenol | (Alkaline | | |
| | test (Benedict | Saponin test | H₂SO₄(aq), | (FeCl₃ | reagent | | Proteins |
| | test) Sample + | (Foam test) | shake and | test) | test) | | (Biurest |
| | Na₂CO₃ + | Aqueous | allow to | sample + | Sample + | | test) |
| | Na₃C₆H₅O₇ + | sample shaken | stand | FeCl₃ | NaOH | Amino acids | Sample + |
| | CuSO4 + heat | Results: | Results: | Result: | Results: | (Xanthoproteic | 2% CuSO4 + |
| | Result: | foam produced | golden | Bluish | intense | test) Sample + | 95% ethanol + |
| Test/ | Orange | persisted | yellow | black | yellow | HNO₃ Results: | KOH Result: |
| Sample | red ppt | for 10 mins | color | color | color | Yellow color | Pink color |
| Crude | No | Yes | Yes | No | Yes | Yes | Yes |
| protein | | | | | | | |
| extract | | | | | | | |
| HKUOT- | No | No | No | No | No | Yes | Yes |
| D3 | | | | | | | |
| HKUOT- | No | No | No | No | No | Yes | Yes |
| P1 | | | | | | | |

### Example 3—Characterization of HKOUT-S2 Protein

The novel HKUOT-S2 was characterized for downstream applications by different techniques such as molecular weight determination using Mathematical model, silver staining, mass spectrometry, de novo peptide sequencing and N-terminal sequencing.

### HKUOT-S2 Molecular Weight was Predicted by Mathematical Model

The high-resolution size-exclusion chromatography (SEC) functions to separate molecules in solutions based on their sizes or molecular weights. ^{[42]}To determine the molecular mass of HKUOT-S2 protein, the column used in the SEC was calibrated with proteins of known molecular weights. After protein elution, the column and elution information were used to draw the standard curve for the known proteins. The standard curve linear equation (y=mx+c) and Kav equation, ^{[35, 42]}were used to predict the molecular weight of HKUOT-S2 which ranged from 32.9-34.8 kDa (Table 2).

TABLE 2
Prediction of HKUOT-S2 molecular weight by mathematical model

| | Average | | | | | | |
|---|---|---|---|---|---|---|---|
| | elution | Elution | | Column | | | |
| | time | vol, Ve | Void vol, | vol, Vc | | | Predicted |
| Sample | (min) | (ml) | V0 (ml) | (ml) | Kav | LogMW | MW (kDa) |
| HKUOT- | 28.080 | 11.232 | 8.000 | 24.000 | 0.202 | 4.5410 | 32.9-34.8 |
| S2 | | | | | | | |

### HKUOT-S2 Molecular Mass was Determined by Silver Staining and Mass Spectrometry

To further confirm HKUOT-S2 molecular weight, HKUOT-P1(P1) and HKUOT-S2 (S2) proteins were run in 15% sodium dodecyl sulphate-polyacrylamide gel electrophoresis (SDS-PAGE) followed by silver staining to visualize the protein bands therein against the molecular marker (FIG. 17C). The silver staining results indicated a single band of HKUOT-S2 with molecular weight around 32 kDa (FIG. 17C). The HKUOT-P1 had two close bands (lower and upper bands). The lower band of HKUOT-P1 has similar molecular weight as that of HKUOT-S2 (FIG. 17C). Next, the HKUOT-S2 protein band in the 15% SDS-PAGE was excised and subjected to mass spectrometry for precise molecular weight determination. The mass spectrometry confirmed HKUOT-S2 molecular weight to be 32.22 kDa (FIG. 17D).

### HKUOT-S2 is a Novel Protein

Matrix-assisted laser desorption/ionization mass spectrometry (MALDI-MS) analysis was done to identify the HKUOT-S2 protein. The HKUOT-S2 peptides did not map to any reference protein database. Hence, the HKUOT-S2 protein was subjected to high resolution liquid chromatography tandem mass spectrometry (LC-MS/MS) analysis for de novo peptide sequencing. The results show that the de novo derived peptides have high scores which showed correctly sequenced strings. Two analyses approach were used to identify the target HKUOT-S2: 1) Conventional protein database analysis approach was employed to match the HKUOT-S2 protein sequences against the entire plant kingdom protein sequences downloaded from Uniprot but there was no significant peptide match, 2) Targeted de novo sequence approach was used to confirm the novelty of the HKUOT-S2 protein. The NCBInr Dioscorea genus protein database was used for the matching. NCBI protein basic local alignment search tool (BlastP) results with E-value<0.001, were considered significant and used as inclusive criteria for alignments of high quality. From this analysis, several candidates de novo peptides were identified. However, BlastP of HKUOT-S2 de novo peptide sequences validation approach against Dioscorea spp. also resulted in no significant protein match. Hence, it was concluded that the HKUOT-S2 is potentially a novel candidate protein. The LC-MS/MS results also revealed that HKUOT-S2 has unique de novo peptide sequences such as KTVSLPR (SEQ ID NO: 81), KGNLLECDGGNTAQMMAR (SEQ ID NO: 82), TKSSLPGQTK (SEQ ID NO: 83) and KEVSLPR (SEQ ID NO: 84) (FIG. 17E-17H, Table 3). Analysis of these de novo sequences on PEAKS Studio X-Pro also revealed the peptide intensities and m/z ratios (FIG. 17E-17H, Table 3).

### N-Terminal Sequencing of HKUOT-S2 by Edman Degradation Chemistry

Finally, N-terminal sequencing analysis of HKUOT-S2 was performed to further identify its N-terminal peptide sequences. Here, HKUOT-S2 protein run in 15% SDS-PAGE was transferred to polyvinylidene difluoride (PVDF) membrane. The amino acid residues on the PVDF membrane were cleaved off one at a time and identified by chromatography. The resultant N-terminal sequence of HKUOT-S2 is IKITTYRQ (SEQ ID NO: 86) (Mw=1022.55 Da) corresponding to the three-letter sequence Ile-Lys-Ile-Thr-Thr-Tyr-Arg-Gln (SEQ ID NO: 86) (Mw=1022.55 Da).

### Example 4—In Vivo Studies

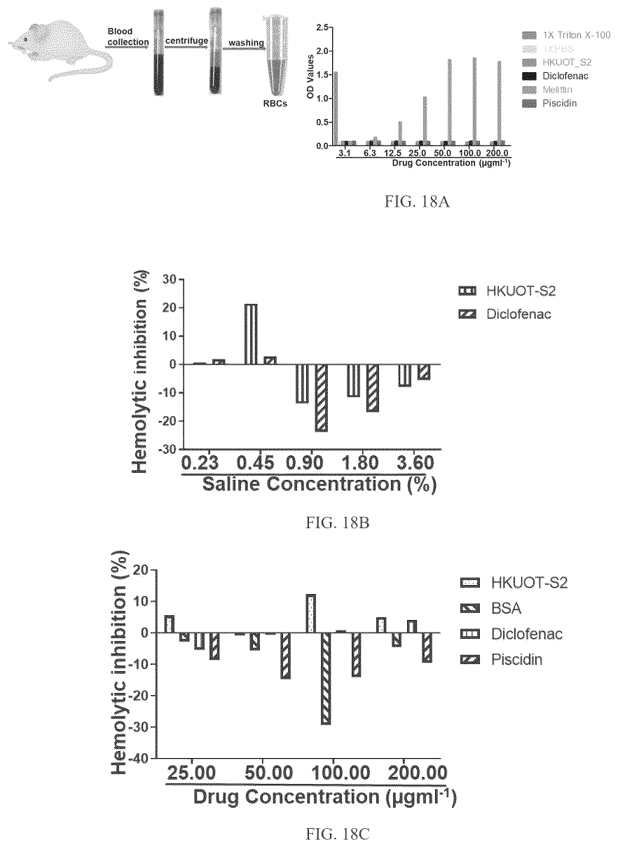

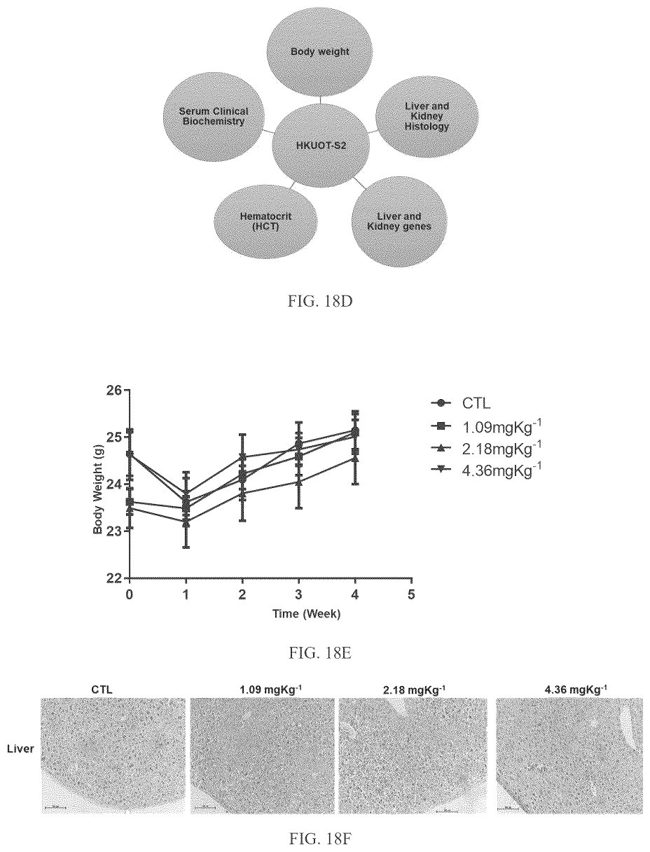

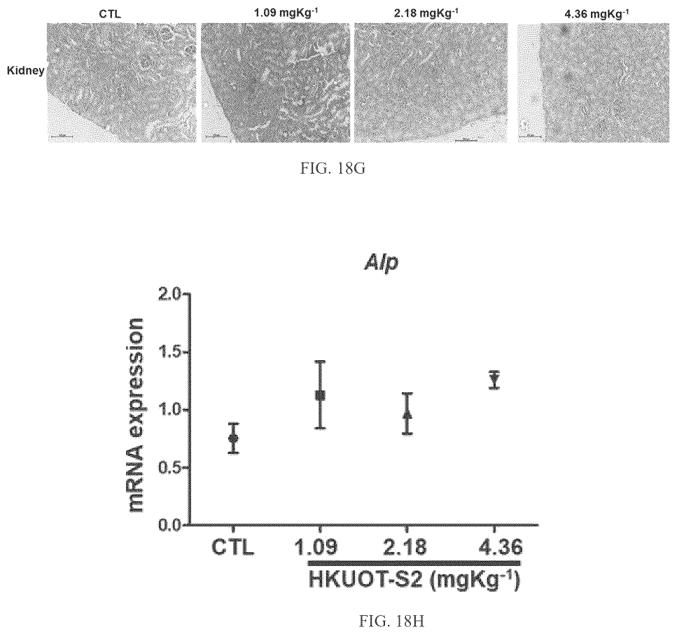

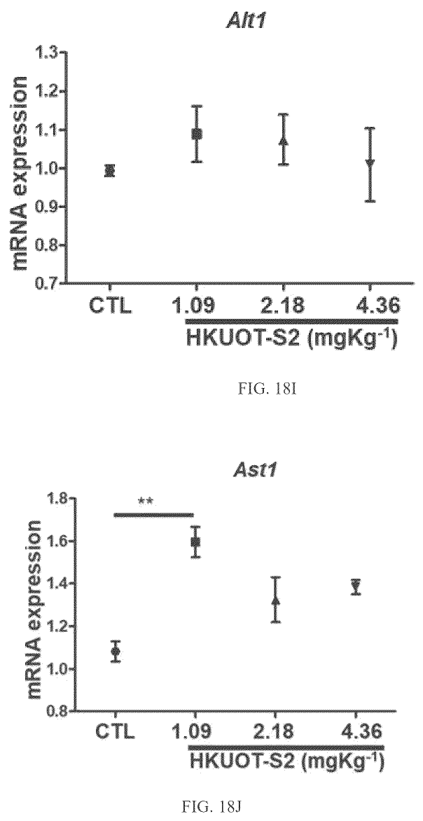

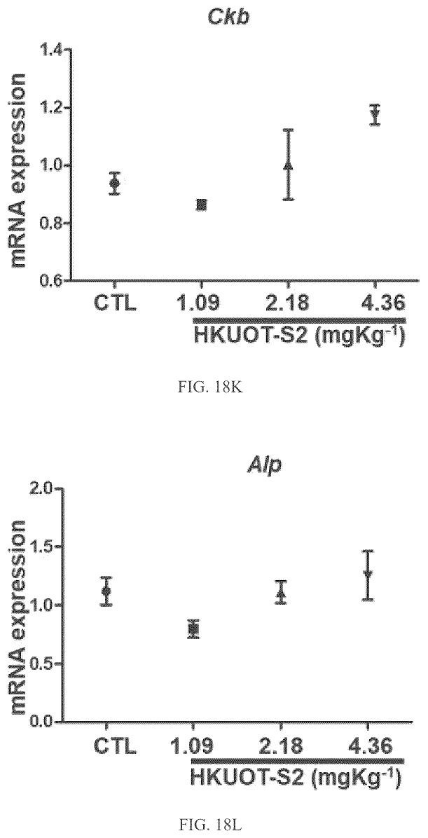

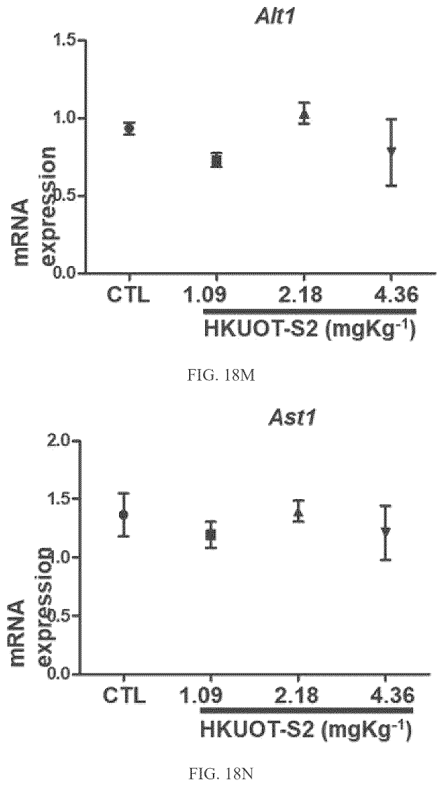

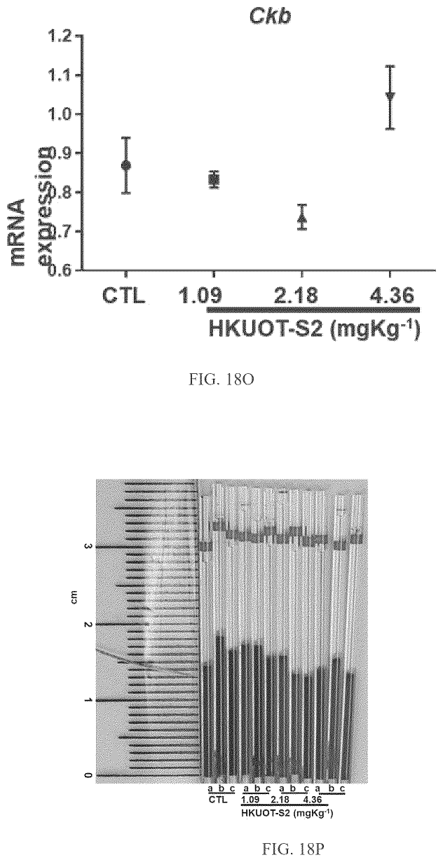

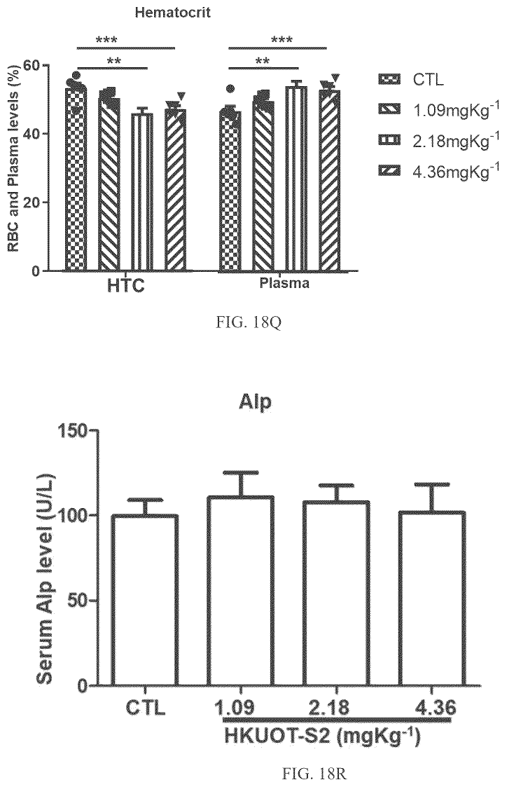

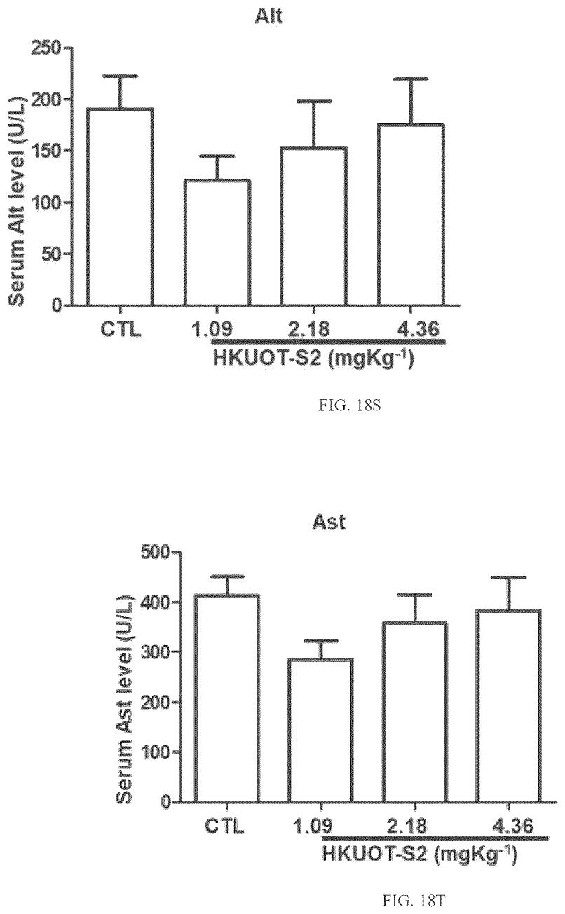

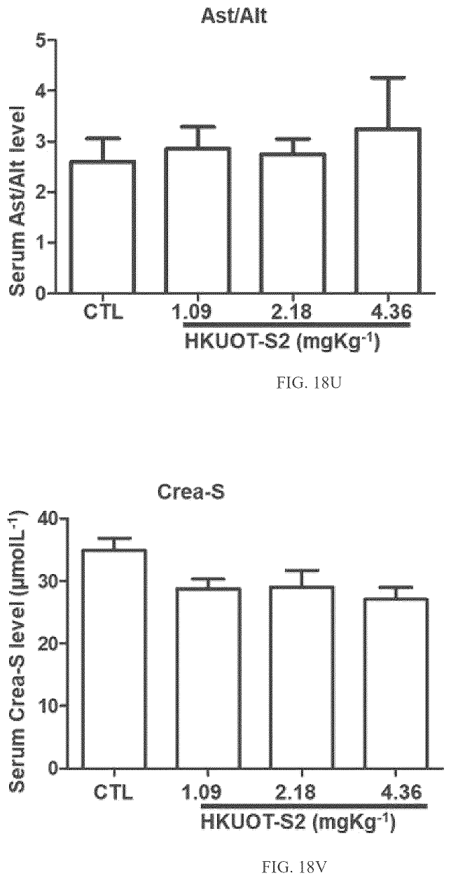

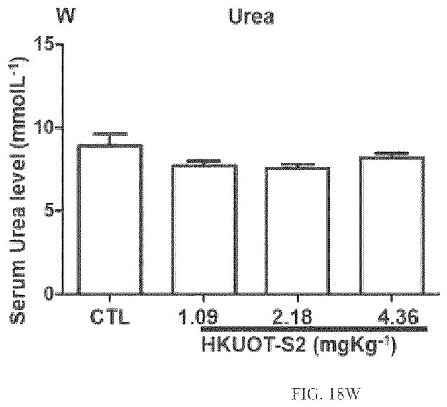

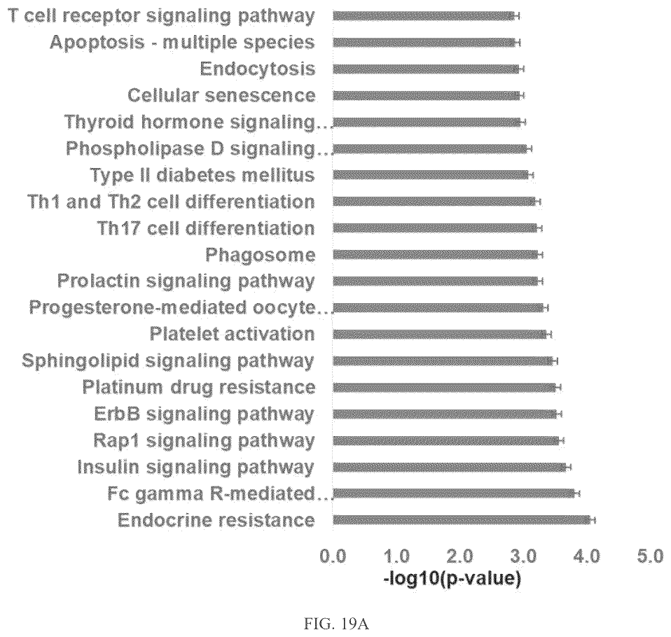

Prior to the in vivo application of HKUOT-S2 protein in bone defect repairs, a comprehensive acute toxicity study of the HKUOT-S2 was perfumed to ensure safe application of this novel protein in vivo. It was confirmed that the therapeutic doses of HKUOT-S2 used in this research have no toxic effects on the mice (FIGS. 18A-18W).

### Example 5—HKOUT-S2 Significantly Enhanced Bone Defect Repairs In Vivo

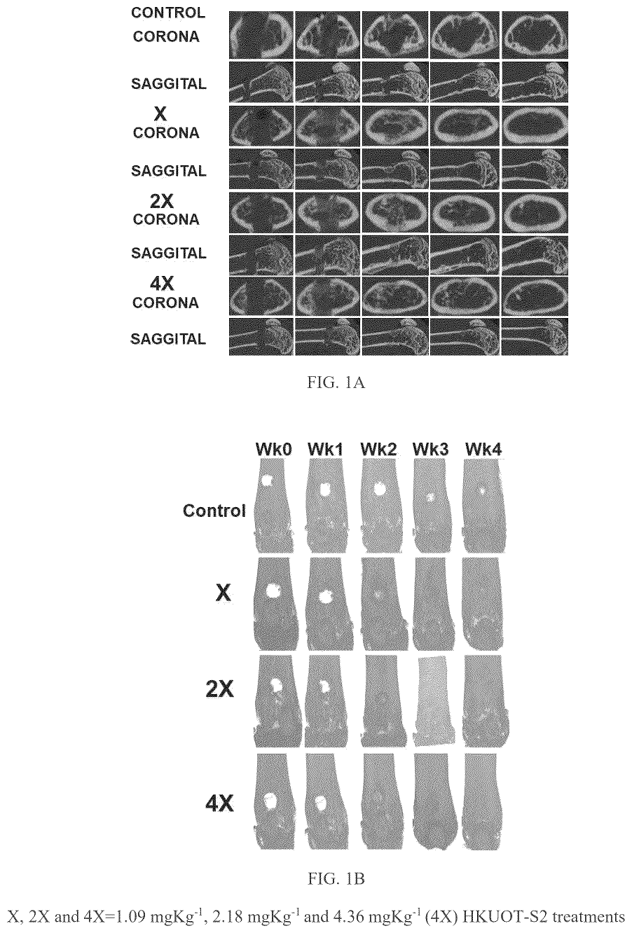

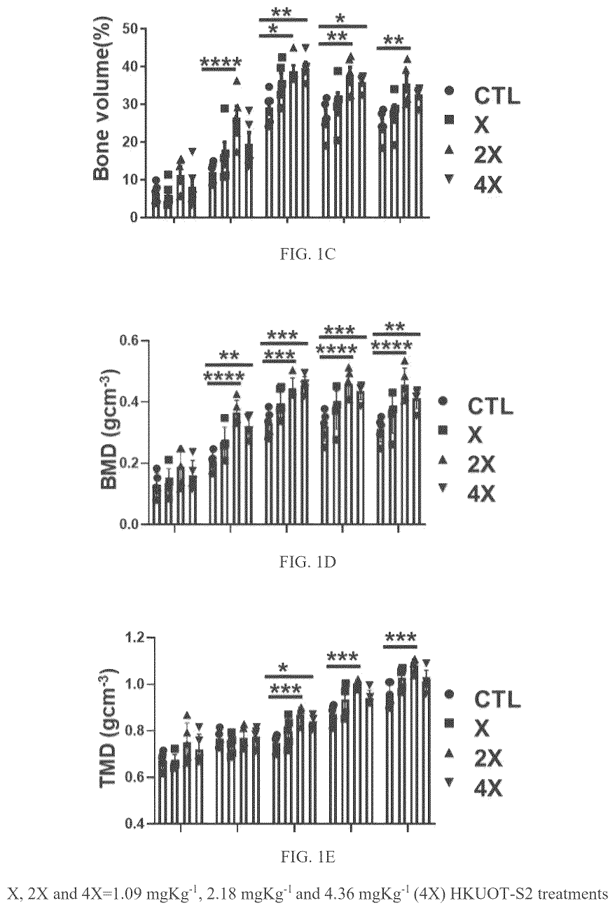

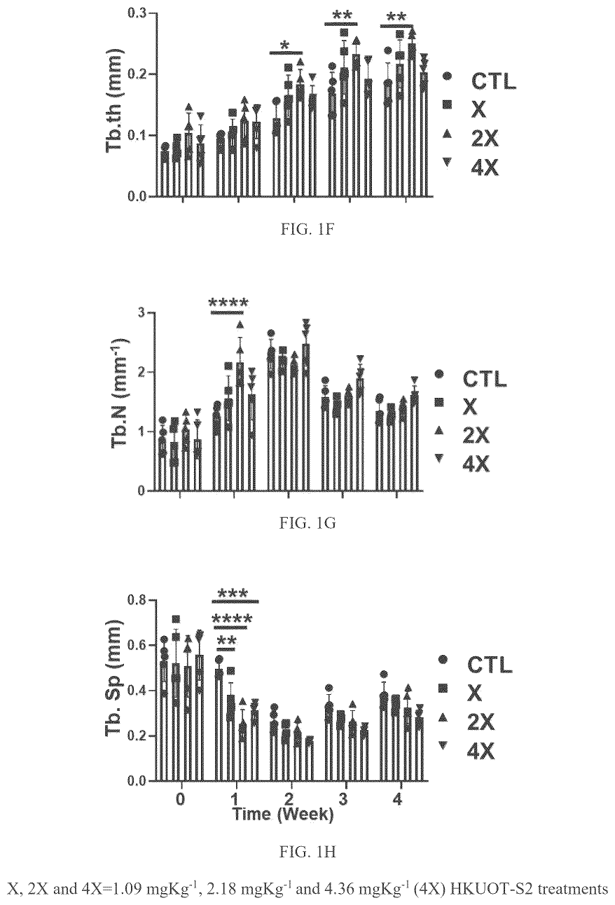

It was demonstrated that Dioscorea spp. proteins such as diosgenin stimulate osteoblasts differentiation in vitro. ^{[35]}The study of Dioscorea spp. proteins in bone fracture healing in vivo has not been fully explored. Here, bone defect model was used to investigate HKUOT-S2 bone defect repairing potentials. A circular hole was drilled in the mice femurs followed by HKUOT-S2 treatments for 4 weeks. The weekly micro-computed tomography (pCT) scan analysis showed that HKUOT-S2 protein promoted bone defect healing in vivo (FIGS. 1A-1B). Quantification of the μCT scanned images using the CTAn software revealed that 2.18 and/or 4.36 mg kg⁻¹ HKUOT-S2 treatments progressively and significantly increased bone volume (BV/TV), bone mineral density (BMD), tissue mineral density (TMD), trabecular thickness (Tb.th), trabecular number (Tb. N), bone surface area to tissue volume (BS/TV) ratio but decreased trabecular separation (Tb. Sp) with no significant effects on bone surface area to bone volume (BS/BV) ratio (FIGS. 1C-1J). These results demonstrated that HKUOT-S2 treatments significantly enhanced bone defect repairs in vivo.

### Example 6—Fluorochrome Labelling Revealed that HKOUT-2-Induced Bone Defect Repairs

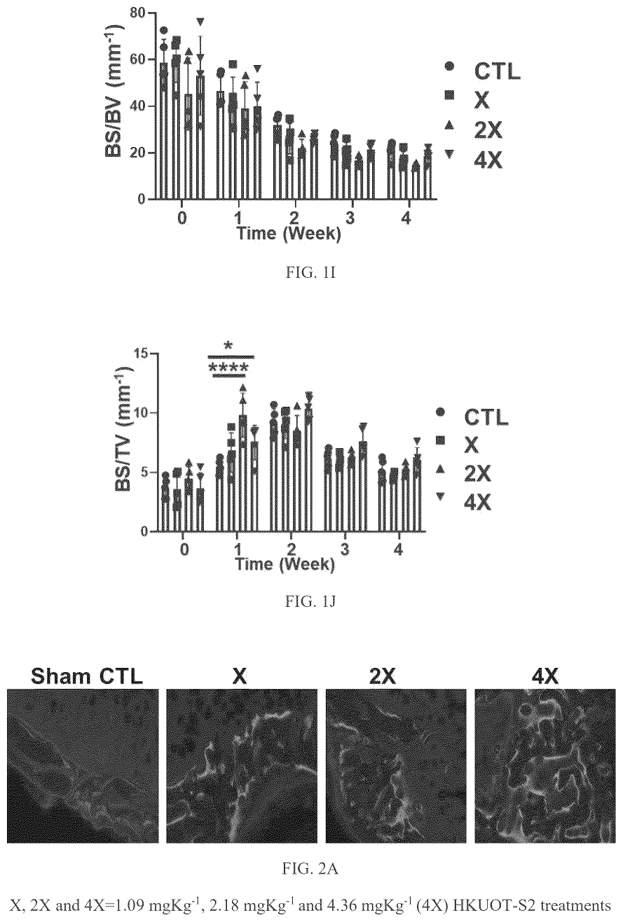

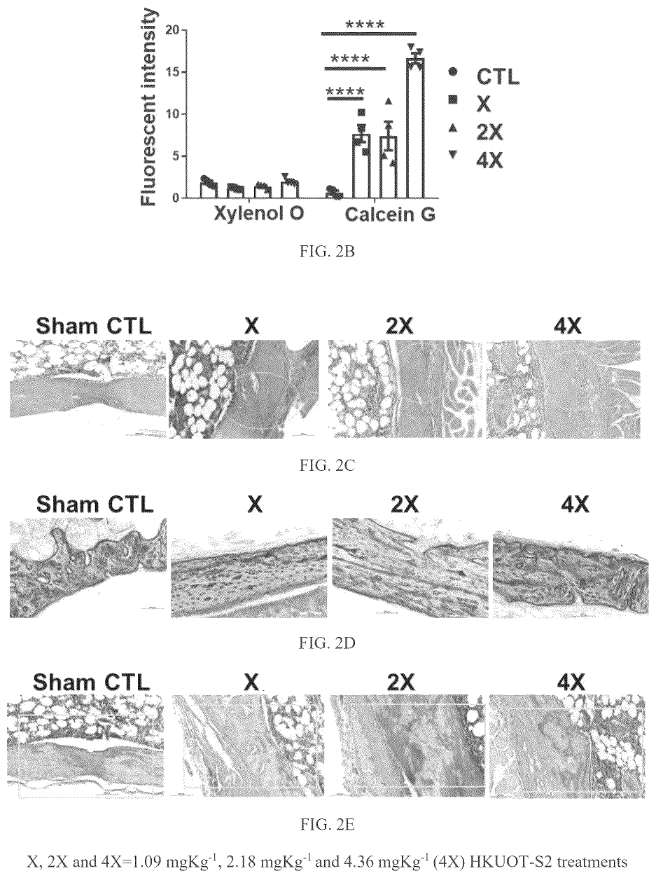

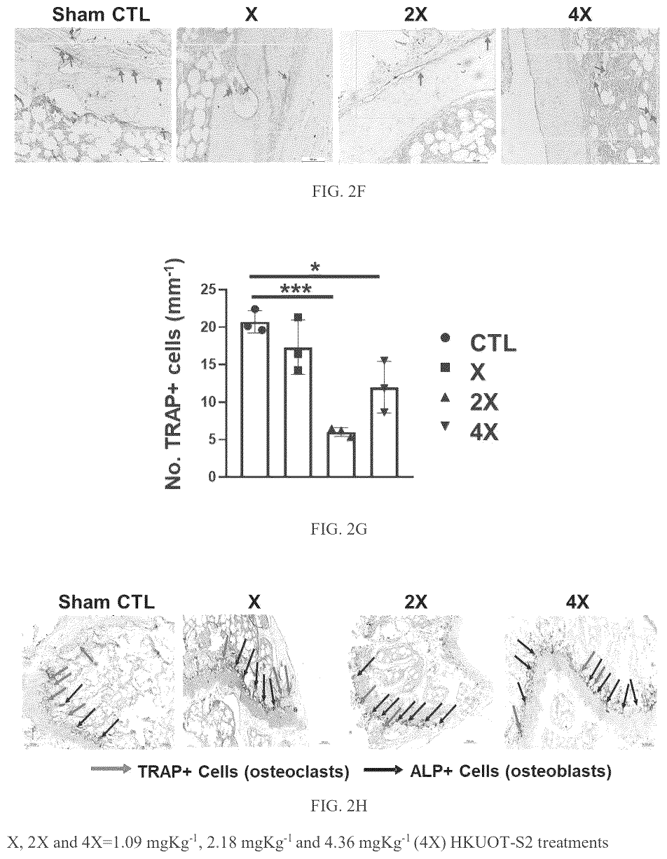

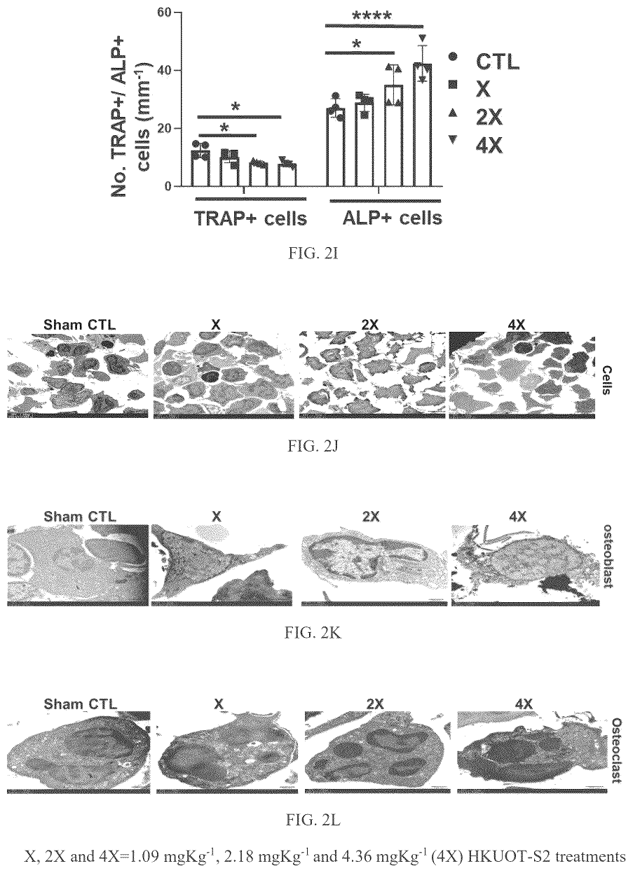

To monitor the HKUOT-S2-induced new bone formation during bone defect repairs, the sham control and HKUOT-S2 treated mice were injected with calcein green (calcein G) and xylenol orange (xylenol 0) on day 19 and 26 post-surgery respectively. The mice were sacrificed on day 29 post-surgery. The harvested femurs with bone defects were fixed, processed, and embedded in methyl methacrylate (MMA) plastic blocks. Sections of the MMA embedded bone tissues were processed for confocal fluorescence microscopy. The fluorescence microscopy analysis showed that the fluorochrome intensities of the calcein G at the bone defect sites were significantly higher in the HKUOT-S2 treatment groups compared with that of the sham control (FIGS. 2A-2B). The results showed that HKUOT-S2 treatments promoted new bone formation.

### Example 7—Histological Analysis Showed that HKOUT-2 Promoted Bone Defect Repairs

To evaluate the effects of HKUOT-S2 on bone defect repairs, the harvested bones were fixed, paraffin embedded, processed for hematoxylin and Eosin (H&E), and Masson-Goldner trichrome staining. The MMA embedded bone tissues were processed for Giemsa staining. Observation of the H&E-stained samples under the light microscope revealed that HKUOT-S2 treatments repaired the bone fractures in the femurs (yellow oval shames) within 4 weeks post-surgery compared to that of the sham controls, (FIG. 2C). The bone tissue and collagen fibers appeared pale pink under the Giemsa staining (FIG. 2D). The collagen fibers at the bone defect sites appeared well organized in the HKUOT-S2 treatment groups indicating that the bone defect healings were almost completed (FIG. 2D). One of the major characteristics of Masson-Goldner trichrome staining is that the immature (unmineralized) and mature (mineralized) bone tissues stain green and red respectively. ^{[43]}The bone defect sites of the HKUOT-S2 treatment groups were stained redder (mature bone matrix) than that of the sham control under the Masson-Goldner trichrome staining indicating that HKUOT-S2 treatments induced bone mineralization (FIG. 2E). As bone mineralization is a function of osteoblasts, it could be deduced that HKUOT-S2 treatments enhanced osteoblast activities to promote bone defect repairs.

### Example 8—HKUOT-2 Enhanced Bone Defect Repairs Via Modulation of Osteoclast and Osteoblast Activities

Bone remodeling involves sequentially modulated osteoclast-induced bone resorption and osteoblast-stimulated bone formation. ^{[18]}Osteoblast activities generally outweigh osteoclast activities to favor new bone formation. We therefore hypothesized that HKUOT-S2 treatments could decrease osteoclast activities but increase osteoblast functions to enhance new bone formation. To test this hypothesis, 4 μm sections of the paraffin embedded bone tissues were subjected to TRAP and ALP double immunostaining. The results revealed that the 2.18 and 4.36 mg kg⁻¹ HKUOT-S2 treatments significantly decreased the number of TRAP+ cells (red arrows, osteoclasts) at the bone defect sites compared to the sham control and the 1.09 mg kg⁻¹ HKUOT-S2 treatment groups (FIGS. 2F-2G). As expected, the ALP+ cells (osteoblasts) were more concentrated at the growth plates (FIG. 2H). The number of both osteoclasts (red arrows) and osteoblasts (black arrows) were quantified at the growth plates (FIGS. 2H-2I). Consistent with the observation at the bone defect sites, 2.18 and 4.36 mg kg⁻¹ HKUOT-S2 treatments significantly reduced osteoclast number but increased osteoblast numbers at the growth plates (FIG. 2I). The immunohistochemistry (IHC) staining results confirmed that HKUOT-S2 treatments modulated osteoclast and osteoblasts activities to enhance bone defect repairs.

### Example 9—Transmission Electron Microscope (TEM) Analysis of Osteoclast and Osteoblast Cytology in the HKUOT-2 Treated Bone Tissues

The IHC results showed that HKUOT-S2 treatment decreased and increased the number of osteoclast and osteoblast respectively to enhance new bone formation. It could be argued that HKUOT-S2 treatment impaired the architectural integrity and the ultrastructure of osteoclasts to reduce their population to favor new bone formation. To address such hypothetical argument, fixed and decalcified bone defect regions of the sham control and HKUOT-S2 treatment groups were processed for TEM. The TEM analysis at low magnifications showed normal cytology and cellular architecture among all the experimental groups. Consistent with the IHC results (FIGS. 2F-2I), there were decreased number of multinucleated osteoclast-like cells, but increased numbers of osteoblast-like cells observed in the 2.18 and 4.36 mg kg⁻¹ HKUOT-S2 treatment groups than in the sham control and the 1.09 mg kg⁻¹ HKUOT-S2 treatment groups (FIG. 2J). TEM analysis of osteoblasts and osteoclasts at high magnifications revealed normal cytological ultrastructure such as cell membranes, endoplasmic reticulum (ER), mitochondria, and nuclei in all the treatment groups (FIGS. 2K-2L). TEM analysis of the osteoblasts and osteoclasts nuclei, however, showed that, the osteoblasts nuclei generally appeared lighter in color (more euchromatic in nature) (FIG. 2K) while osteoclasts nuclei generally appeared darker in color (more heterochromatic in nature) (FIG. 2L) in the HKUOT-S2 treatment groups than that of the sham controls. Euchromatic nuclei are enriched generally active in gene transcriptions while the heterochromatic nuclei are less active in gene transcriptions. HKUOT-S2 treatment could therefore modulate the differential gene transcription potentials of the osteoblasts and osteoclasts to promote new bone formation and defect healings in vivo at the experimental endpoint.

### Example 10—HKOUT-S2 Induced Osteogenic Gene Expressions in the Defective Bones

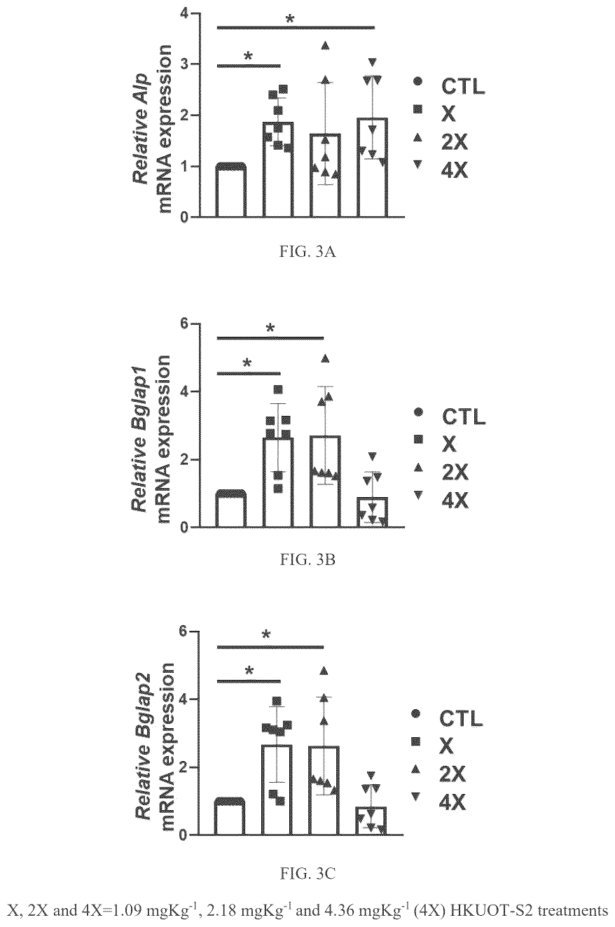

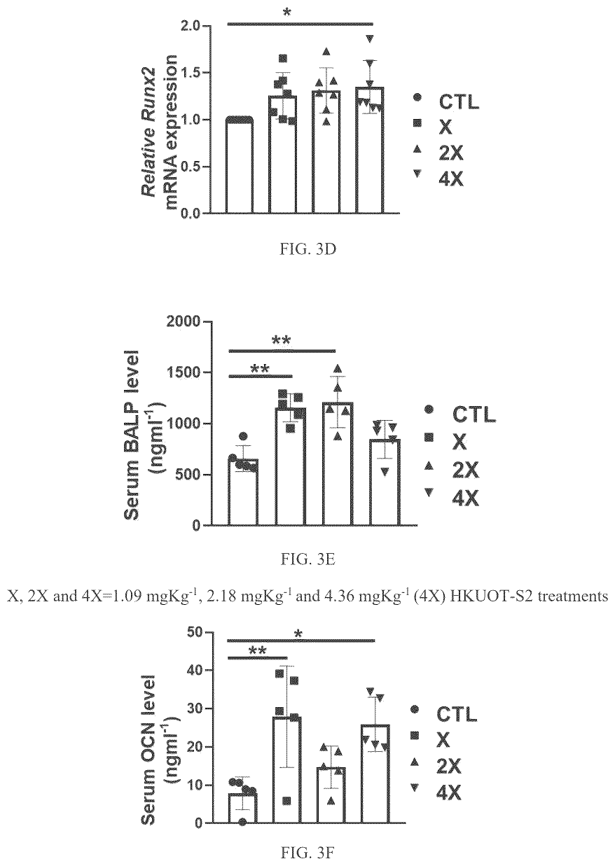

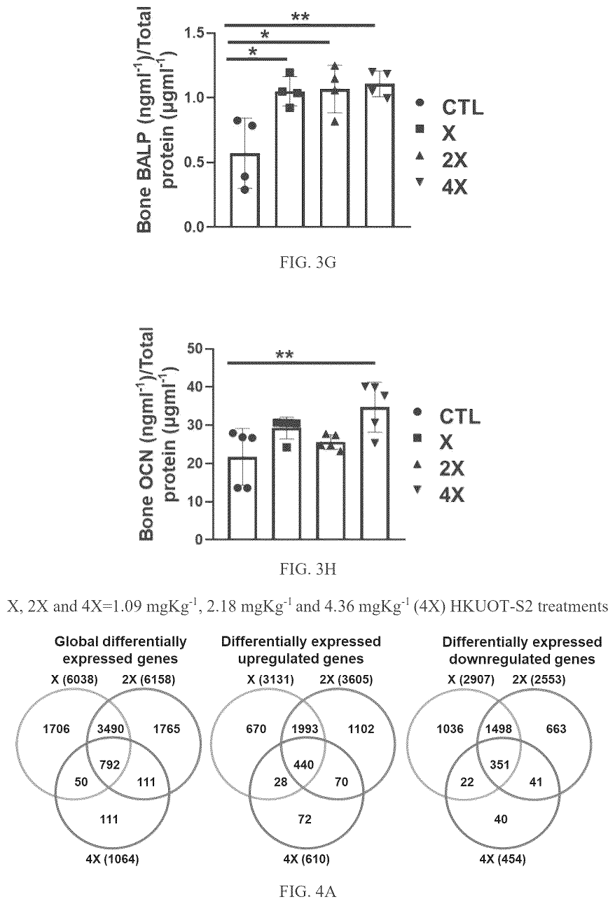

TEM results lead to the hypothesis that HKUOT-S2 could increase osteogenic gene expressions to stimulate bone defect healing. To test this hypothesis, the extracted total RNAs from the surgical femurs were processed for osteogenic gene expression analysis. qPCR results showed that HKUOT-S2 treatments significantly increased Alp, Bglap1, Bglap2 and Runx2 expressions in the defective bones (FIGS. 3A-3D). The results confirmed that HKUOT-S2 treatment generally increased osteogenic gene expressions to enhance bone defect repairs.

### Example 11—HKOUT-S2 Treatment Significantly Elevated BALP and OCN Levels In Vivo

Both the in vitro and in vivo data consistently illustrated that HKUOT-S2 treatments induced osteogenesis by increasing Alp expression. Furthermore, HKUOT-S2 treatments induced Bglap1 and Bglap2 gene expressions in the surgical bone tissues. Both Bglap1 and Bglap2 encode for osteocalcin (OCN) protein. Consequently, it was expected that the bone and/or serum ALP and OCN levels would also be elevated upon HKUOT-S2 treatments. To evaluate the HKUOT-S2-elevated ALP and OCN levels, blood sera and bone lysates from the sham control and HKUOT-S2 treatment groups were processed for enzyme-linked immunosorbent assay (ELISA) analysis using the bone-specific ALP (BALP) and osteocalcin (OCN) ELISA kits. The results demonstrated that HKUOT-S2 treatment significantly increased BALP and OCN levels in both the sera and bone lysates indicating that HKUOT-S2 treatments induced osteoblast activities to enhance new bone formation and defect repairs in vivo (FIGS. 3E-3H).

### Example 12—Transcriptomic Analysis of HKUOT-S2-Induced Bone Defect Healing

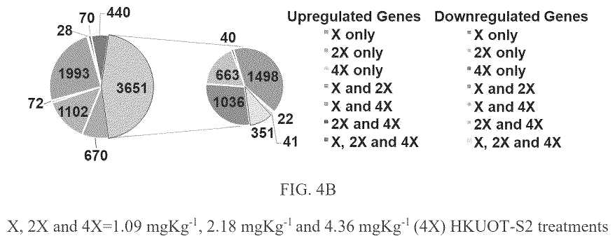

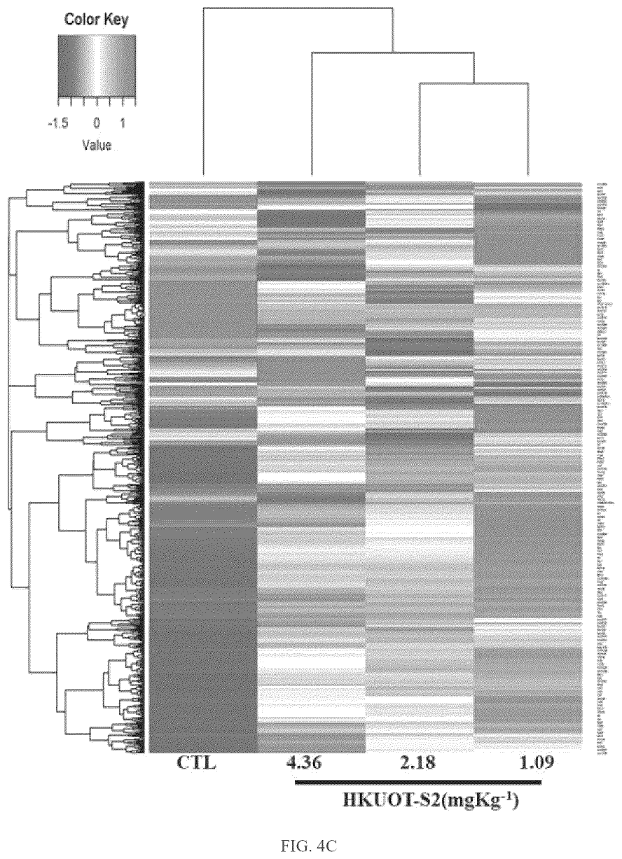

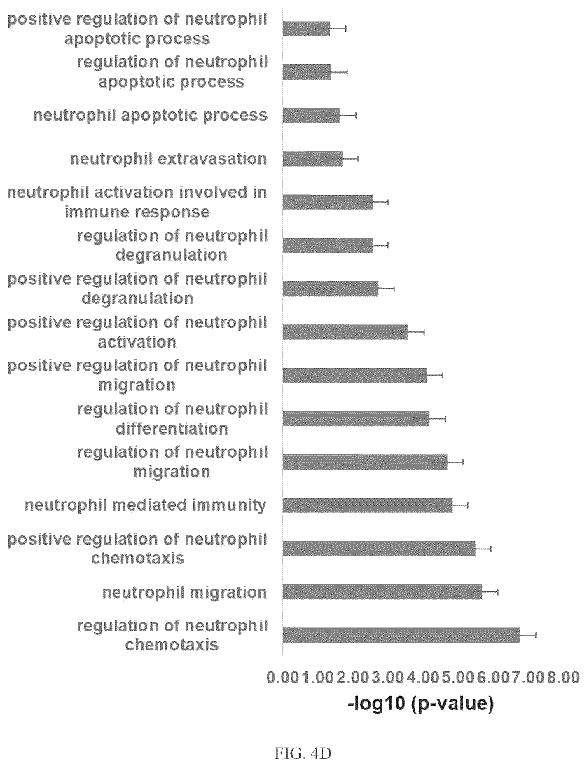

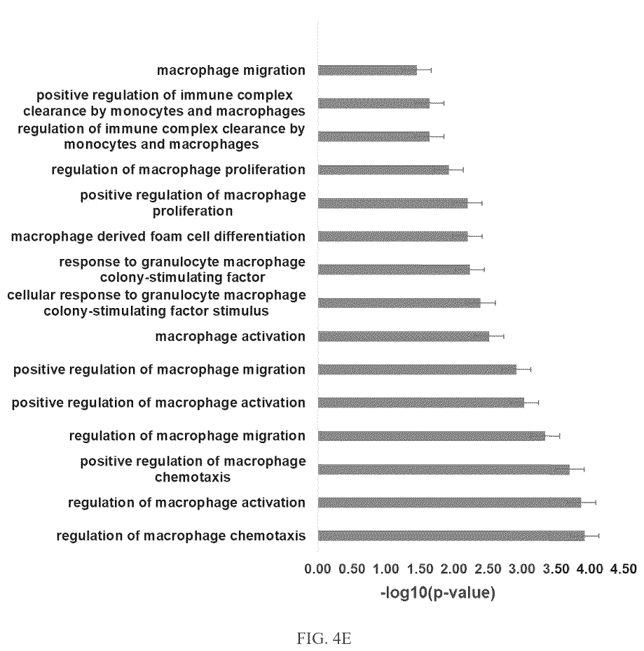

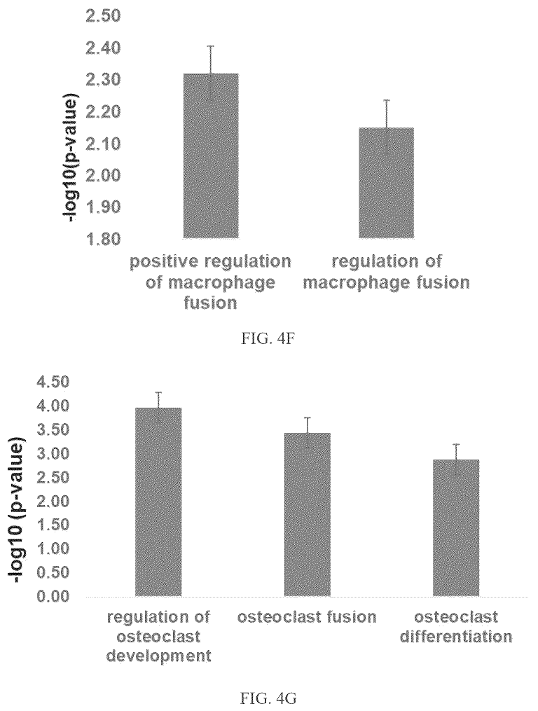

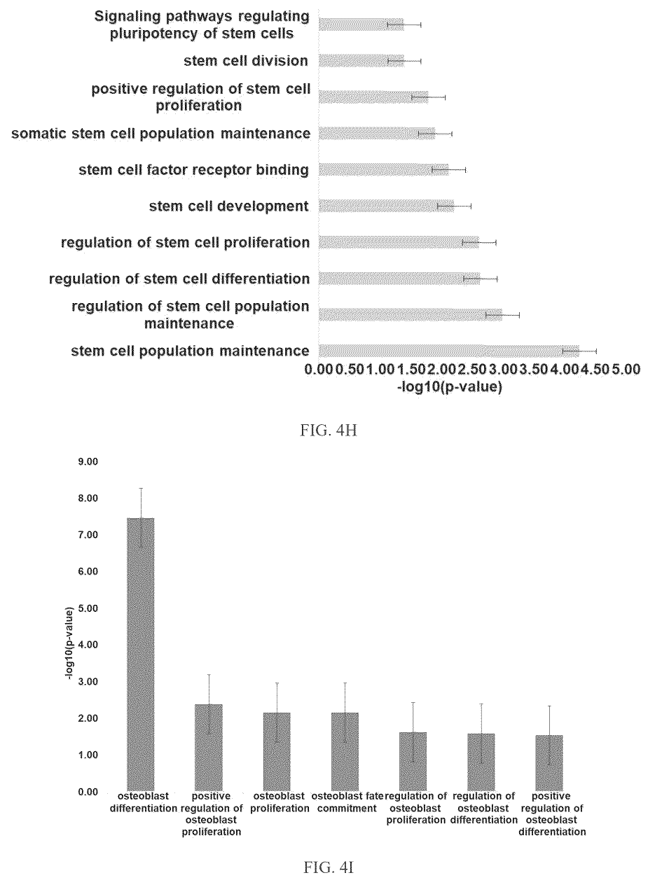

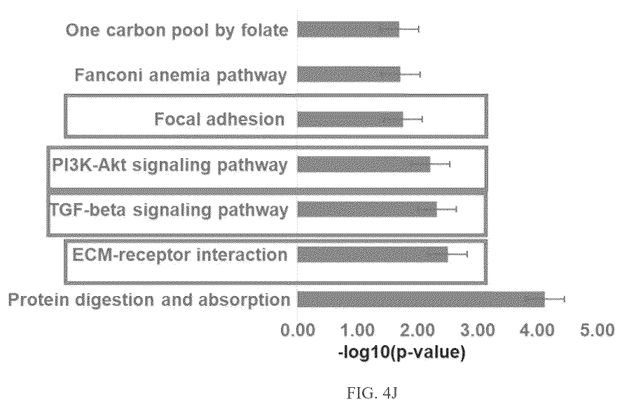

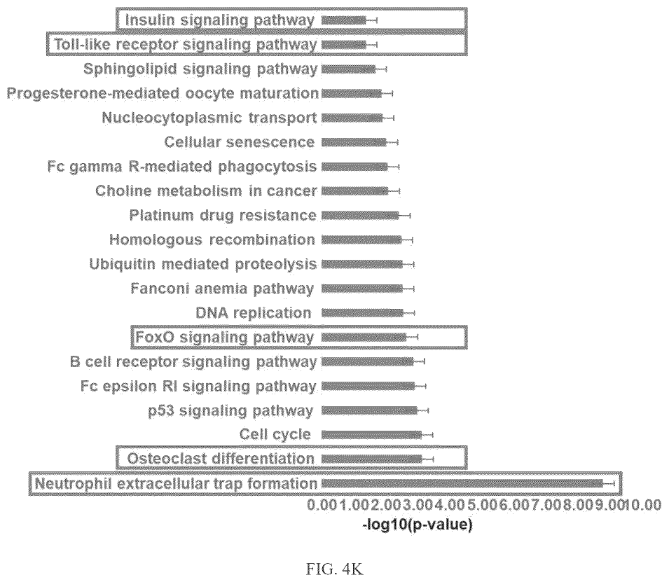

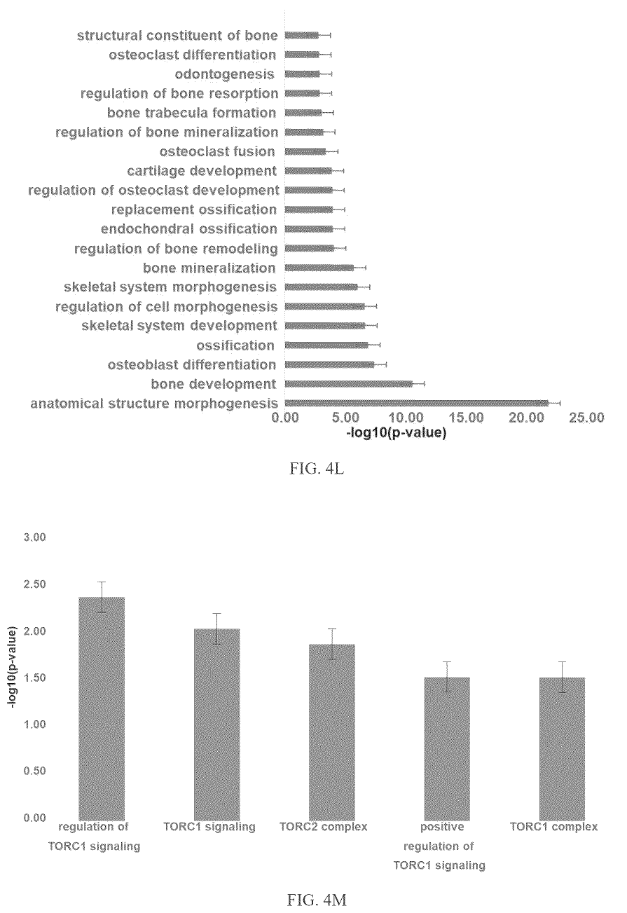

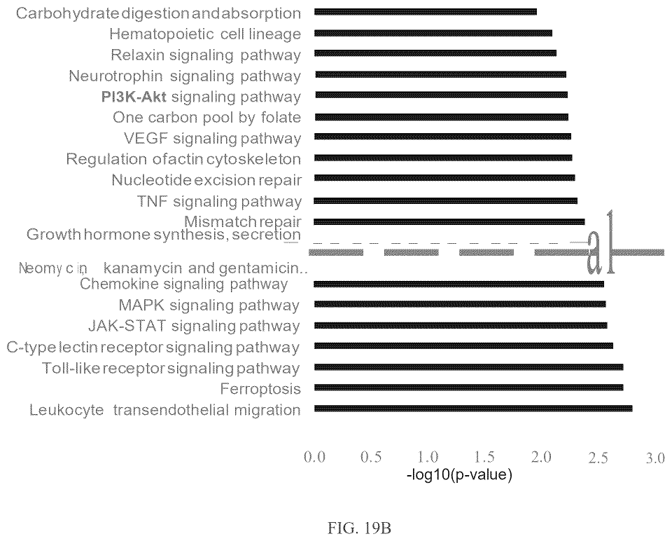

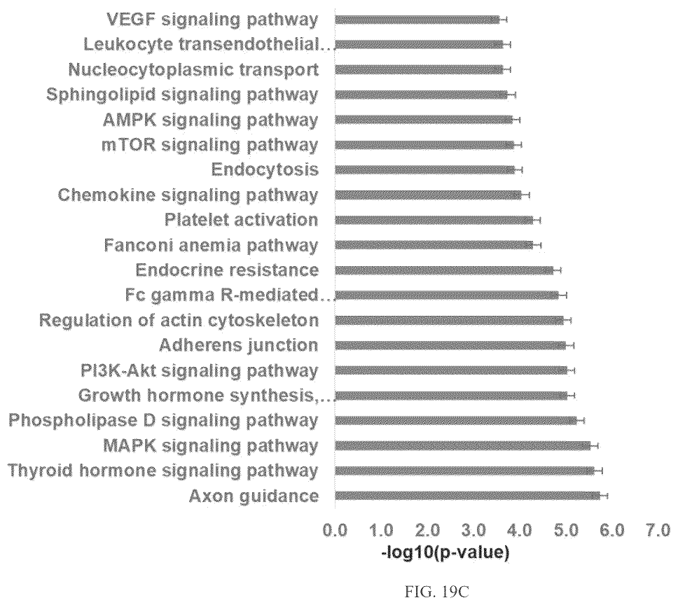

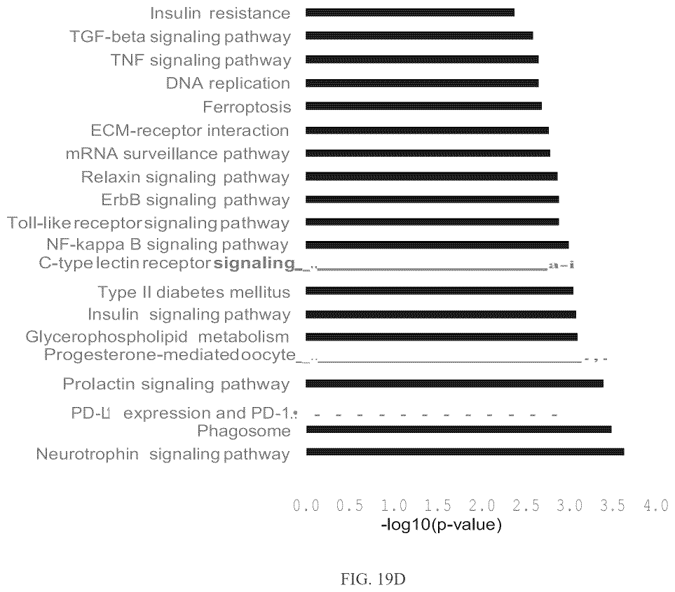

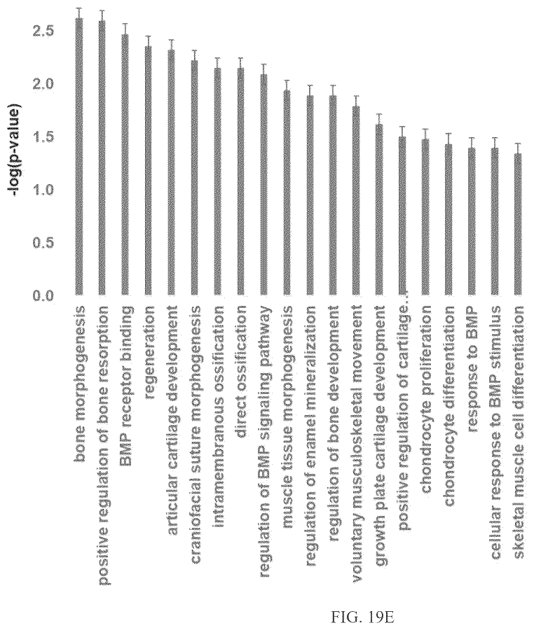

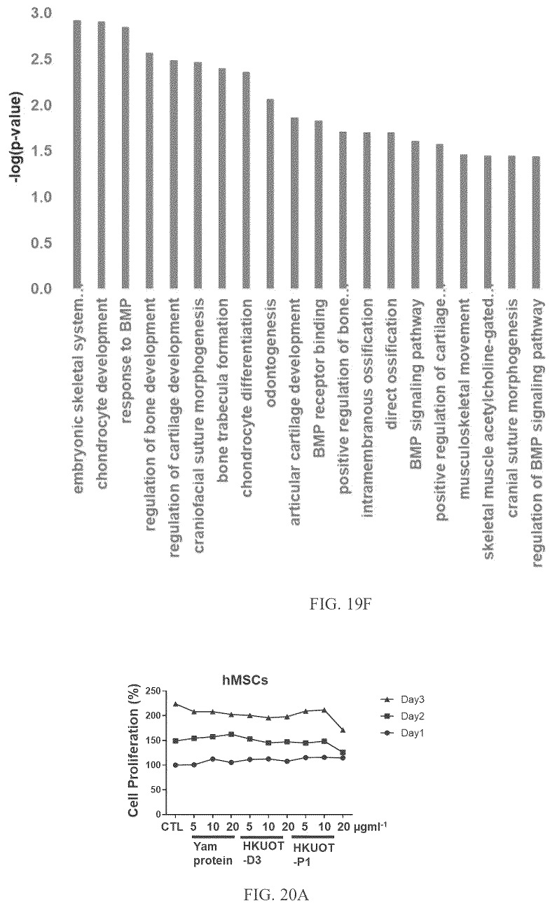

All the current data lead to the hypothesis that HKUOT-S2 treatments could induce differentially expressed genes to facilitate bone defect repairs. To test this hypothesis, high quality RNA extracted from the defective bones of the sham control and the HKUOT-S2 treatment groups were processed for rRNA-depleted RNA-seq. The transcriptomic data analysis revealed that HKUOT-S2 treatments induced differentially expressed genes and transcripts in the defective bones (Table 4, Table 5). Filtering of the differentially expressed genes and transcripts with FDR<0.05 revealed that, 1.09, 2.18 and 4.36 mg kg⁻¹ HKUOT-S2 treatments induced 6038, 6158 and 1064 genes, 9477, 9974 and 2871 transcripts respectively (Table 4). Among the differentially expressed genes, 792 genes were commonly shared by all the three doses, 3490 genes were common to only 1.09 and 2.18 mg kg⁻¹ doses, 111 genes were only found in both 2.18 and 4.36 mg kg⁻¹ doses, 50 genes were common to only 1.09 and 4.36 mg kg⁻¹ doses of HKUOT-S2 treatments (FIG. 4A (left)). 54% of the differentially expressed genes were upregulated whiles 46% of the differentially expressed genes were downregulated (FIG. 4B, Table 5). Also, among the differentially expressed genes, 440 upregulated and 351 downregulated genes were found to be common to all the three doses of HKUOT-S2 treatments (FIG. 4A (middle and right)). Hierarchical clustering of the top 1000 most differentially expressed genes using heatmap also illustrated the transcriptional distances between the sham control and HKUOT-S2 treatment groups (FIG. 4C). The heatmap results revealed that the sham control group was transcriptionally segregated from the HKUOT-S2 treatment groups (FIG. 4C). The HKUOT-S2 treatment groups shared some common transcriptional patterns. Generally, the 1.09 and 2.18 mg kg⁻¹ HKUOT-S2 treatment groups have close transcription distance (FIG. 4C). The experimental groups could therefore be hierarchically arranged in descending order according to their transcriptional segregations as sham control, 4.36, 2.18 and 1.09 mg kg⁻¹ HKUOT-S2 treatment groups. Partek Genomics Suite (PGS) software was then used to analyze the differentially expressed genes with FDR<0.05. HKUOT-S2-induced differentially expressed genes were significantly enriched in the maintenance, proliferation, development, differentiations and functions of neutrophils, monocytes, macrophages, osteoclasts, stem cells stem cells and osteoblasts (FIGS. 4D-4I). Pathway enrichment analysis showed that HKUOT-S2 treatments significantly enriched genes in many Kyoto Encyclopedia of Genes and Genomes (KEGG) pathways (FIGS. 4J, 4K; FIGS. 19A-19D). HKUOT-S2 treatments significantly enriched genes in many KEGG pathways such as AMPK signaling, focal adhesion, foxO signaling, osteoclast differentiations, PI3K-AKT signaling, MAPK signaling, VEGF signaling, TNF signaling, mTOR and TGF-β signaling pathways (FIGS. 4J, 4K; FIGS. 19A-19D). Functional analysis further revealed that HKUOT-S2 treatments significantly enriched genes in gene ontology (GO) terms such as regulation of anatomical structure morphogenesis, osteoblasts, osteoclasts and odontoblasts differentiations, stem cells development and differentiations, intramembranous and endochondral bone formation, trabecular development, bone remodeling, bone and tooth mineralization, odontogenesis musculoskeletal development and movements, cartilage formation and regulation of BMP signaling pathway (FIGS. 4L; 19E-19F). The HKUOT-S2-induced differentially expressed genes were also significantly enriched in mTOR protein complex and signaling pathway (FIG. 4M).

TABLE 5
Percentage of differentially expressed genes

| HKUOT-S2 treatment (mg/Kg) | No. genes | % Of genes |
|---|---|---|
| 1.09 only upregulated genes | 670 | 8.35 |
| 1.09 and 2.18 upregulated genes | 1993 | 24.83 |
| 1.09 and 4.36 upregulated genes | 28 | 0.35 |
| 1.09, 2.18 and 4.36 upregulated genes | 440 | 5.48 |
| 2.18 only upregulated genes | 1102 | 13.73 |
| 2.18 and 4.36 upregulated genes | 70 | 0.87 |
| 4.36 only upregulated genes | 72 | 0.90 |
| 1.09 only downregulated genes | 1036 | 12.91 |
| 1.09 and 2.18 downregulated genes | 1498 | 18.66 |
| 1.09 and 4.36 downregulated genes | 22 | 0.27 |
| 1.09, 2.18 and 4.36 downregulated genes | 351 | 4.37 |
| 2.18 only downregulated genes | 663 | 8.26 |
| 2.18 and 4.36 downregulated genes | 41 | 0.51 |
| 4.36 only downregulated genes | 40 | 0.50 |
| Total | 8026 | 100.00 |

### Example 13—Validation of BMP, AMPK and mTOR Signaling Pathway Related Genes

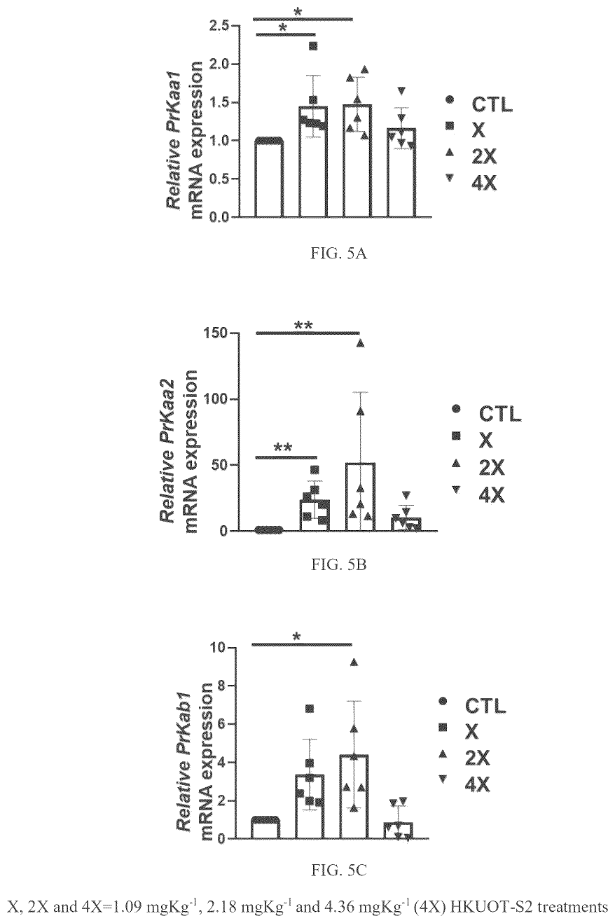

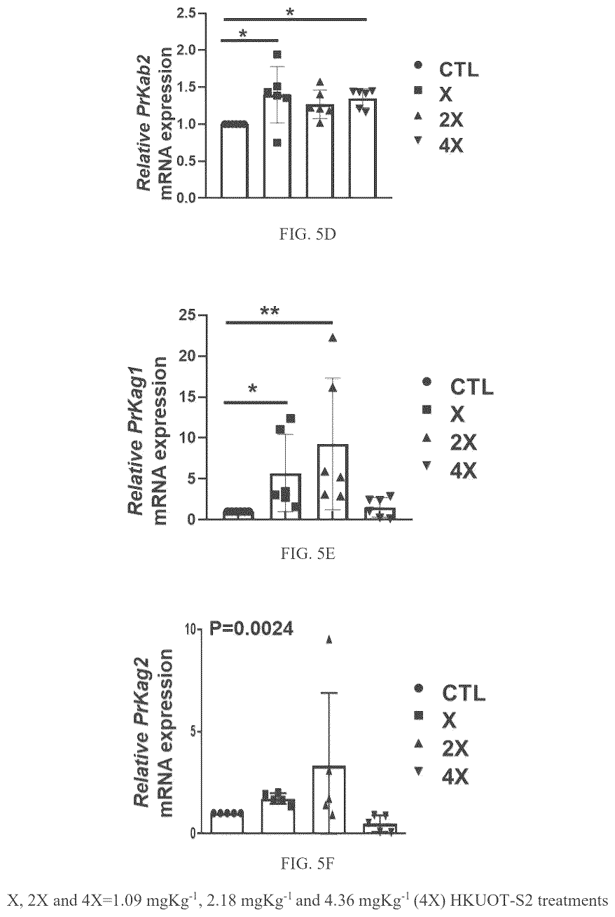

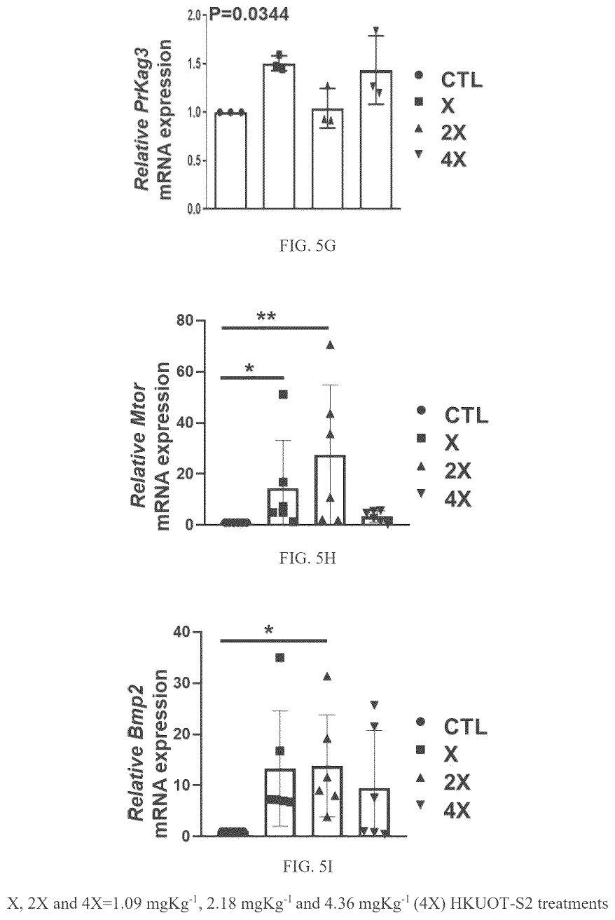

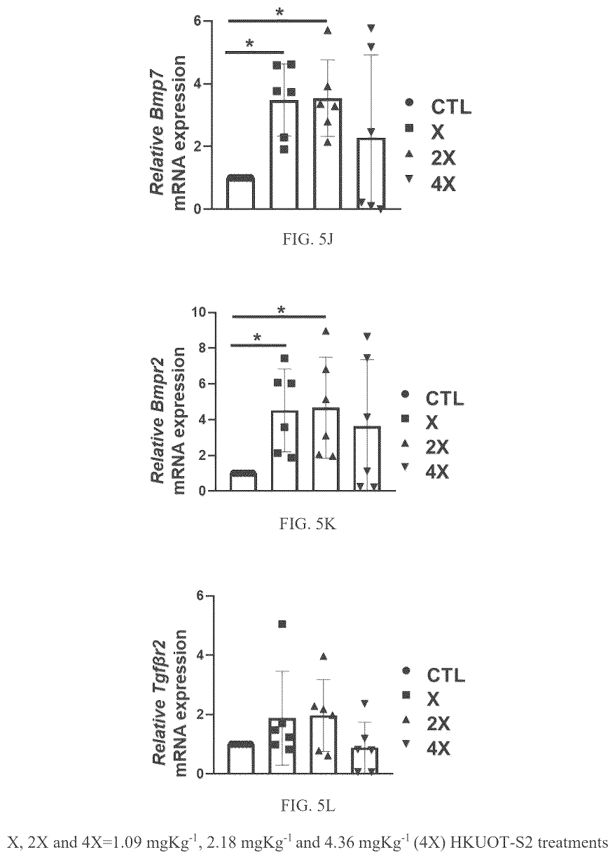

To validate some of the differentially expressed genes related to BMP, TGF-β, AMPK and mTOR signaling pathways, RNAs from the defective bones were subjected to qPCR analysis. The results showed that HKUOT-S2 treatments significantly upregulated AMPK and mTOR related genes such as Prkaa1, Prkaa2, Prkab1, Prkab2, Prkag1, Prkag2, Prkag3 and Mtor in the repaired bone tissues (FIGS. 5A-5H). HKUOT-S2 treatments also significantly increased Bmp2, Bmp7 and Bmpr2 but not Tgfβr2 expressions in the repaired bone tissues (FIGS. 5I-5L). Validation of these HKUOT-S2-induced differentially expressed genes indicated that, HKUOT-S2 might have modulated AMPK, mTOR and BMP signaling pathways to enhance bone defect repairs in vivo.

### Example 14—HKUOT-S2 Enhanced hMSCS-Osteoblast Differentiation

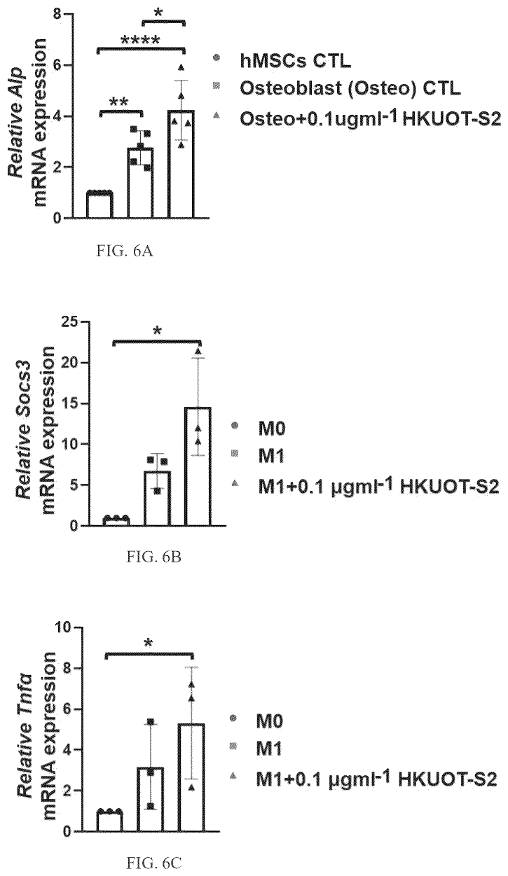

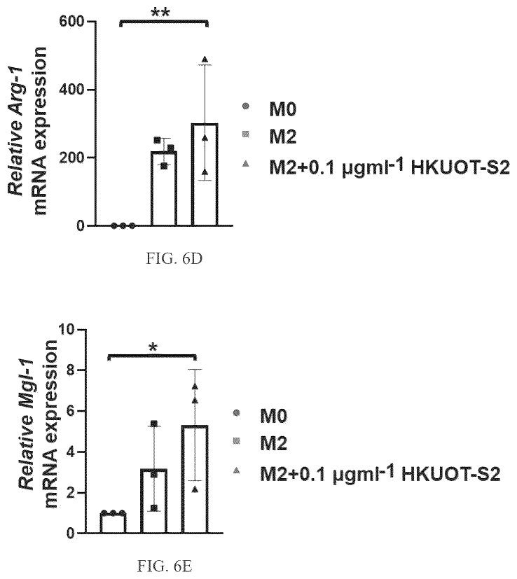

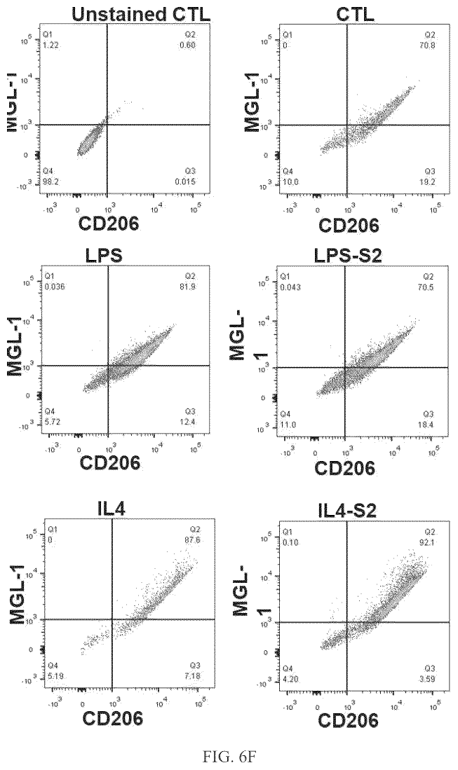

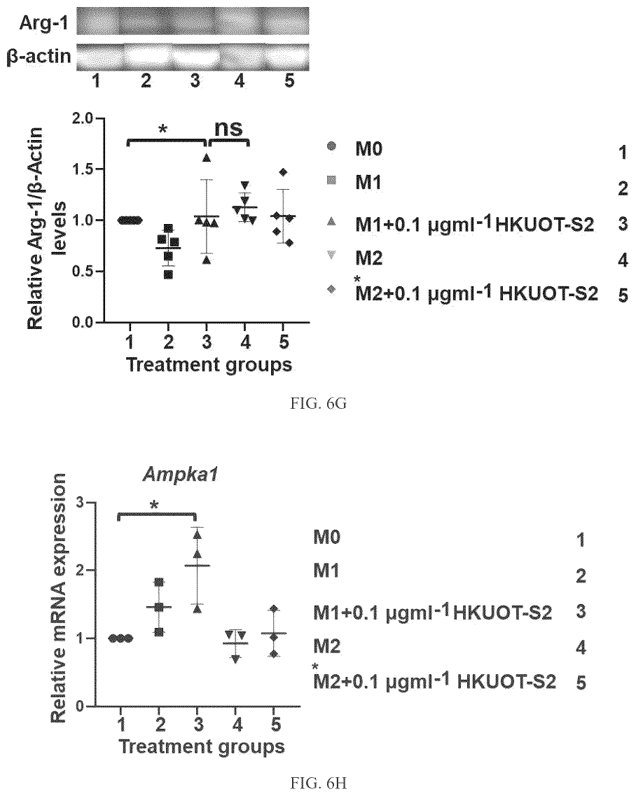

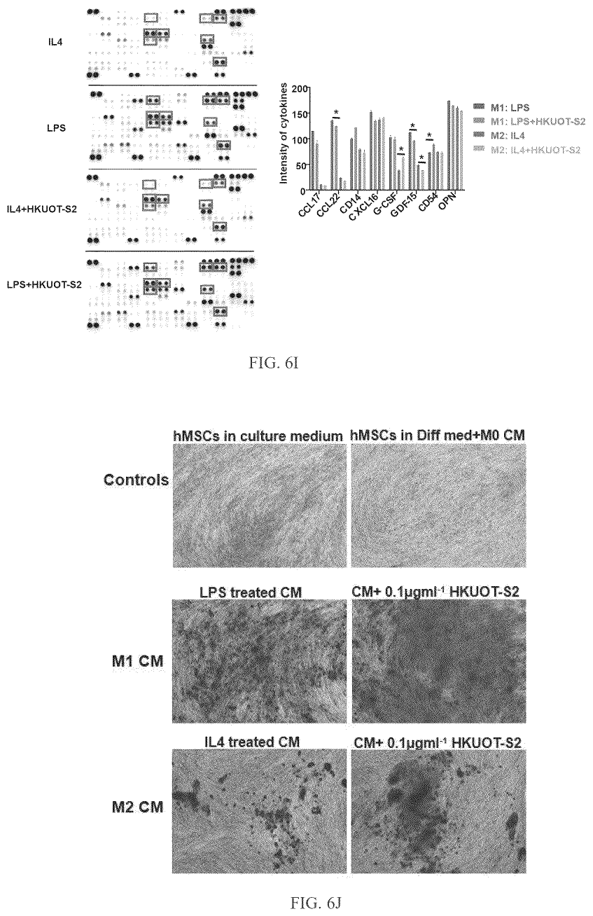

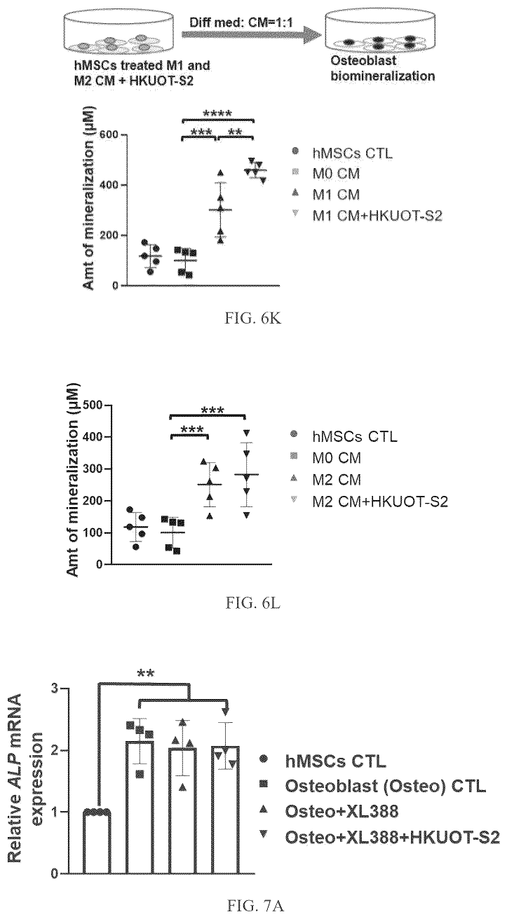

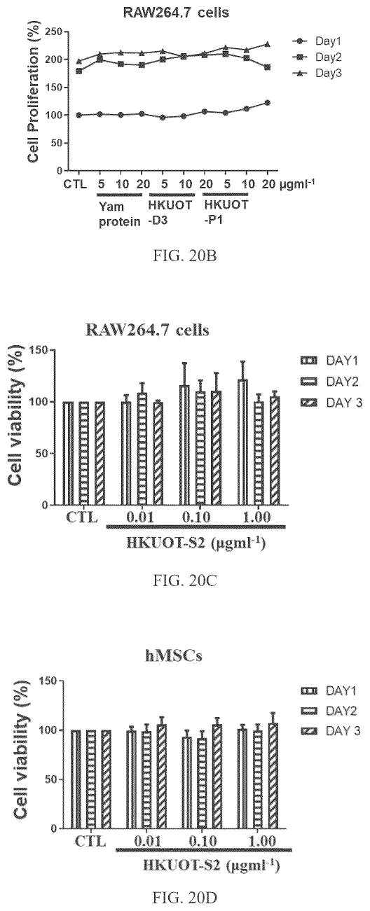

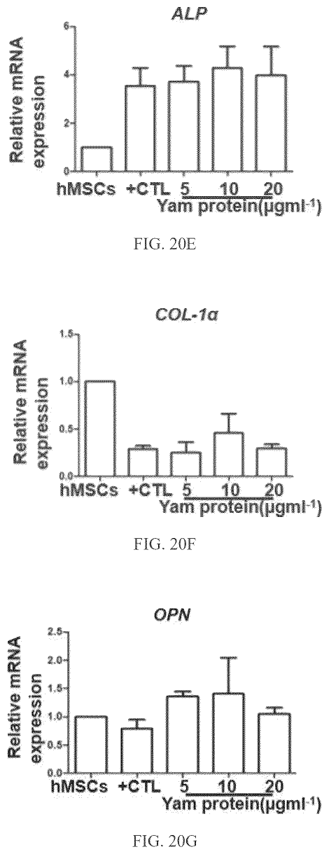

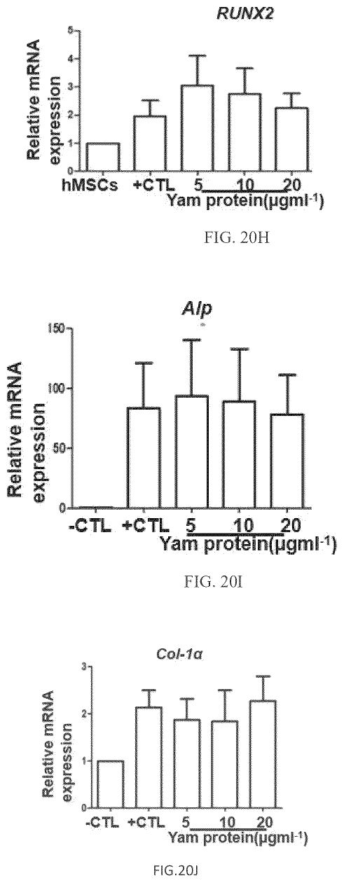

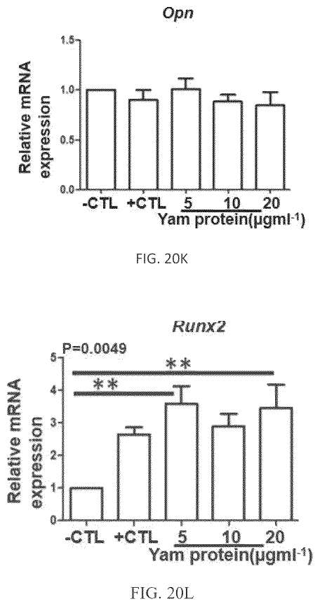

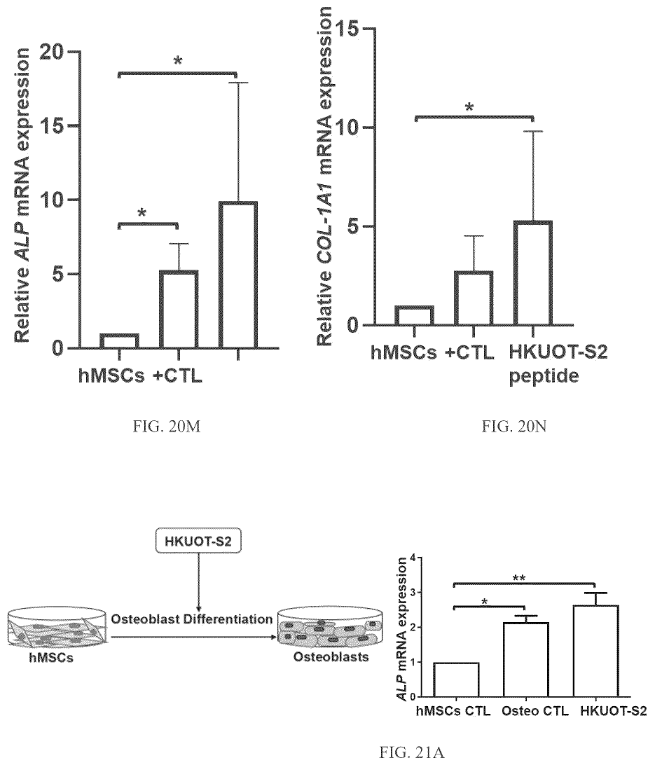

It was demonstrated that extracts from both the root tuber and bark of Dioscorea batatas exhibited osteogenic activity by promoting osteoblast differentiation and functions. ^{[35]}It was confirmed in this research that 5-20 μg ml⁻¹ of the crude protein extract, HKUOT-D3 and HKUOT-P1) and 0.01-1 μg ml⁻¹ HKUOT-S2 had no significant effects on the hMSCs viability (FIGS. 20A, 20D). It was also shown that 5 μg ml⁻¹ of the crude Dioscorea opposita Thunb protein extract exhibited osteogenic activity by increasing RUNX2 expressions in both hMSCs- and MC3T3-E1 cells-derived osteoblasts (FIGS. 20H, 20L). The transcriptome data from the in vivo studies also indicated that HKUOT-S2-induced differentially expressed genes were significantly enriched in the maintenance, proliferation, development, differentiations and functions of stem cells and osteoblasts (FIGS. 4H,4I). To test and confirm osteogenic activities of the HKUOT-S2 protein, hMSCs were differentiated into osteoblasts with or without HKUOT-S2. The quantitative polymerase chain reaction (qPCR) results showed that 0.1 μg ml⁻¹ HKUOT-S2 enhanced hMSCs-osteoblast differentiation by significantly increasing ALP expressions (FIG. 6A). The in vitro result supported the osteogenic activities of HKUOT-S2 to enhance bone defect repairs in vivo.

### Example 15—HKUOT-S2 Enhanced M1 and M2 Macrophage Polarizations

It was reported that Dioscorea villosa extracts elicited anti-inflammatory activities in mice. ^{[44]}Dioscorin from Dioscorea spp. was also shown to trigger immunomodulatory activities in the RAW264.7 cells and mice. [36]It was confirmed in this research that 5-20 μg ml⁻¹ of the crude protein extract, HKUOT-D3 and HKUOT-P1) and 0.01-1 μg ml⁻¹ HKUOT-S2 had no significant effects on the RAW264.7 cells viability (FIGS. 20B, 20C). This research also revealed that HKUOT-S2-induced differentially expressed genes were significantly enriched in the regulatory functions of monocytes and macrophages to promote bone defect healing in vivo (FIG. 4E). Consequently, RAW264.7 cells were subjected to M1 and M2 macrophage polarization in the presence or absence of HKUOT-S2. The results showed that HKUOT-S2 significantly increased M1 macrophage markers, Socs3 and Tnfα, and M2 macrophage markers Arg-1 and Mgl-1 (FIGS. 6B-6E). Additionally, mouse primary M0 (monocytes) macrophage were subjected to M1 and M2 macrophage polarization with or without HKUOT-S2 treatments. The polarized macrophages were co-stained with fluorescent labelled CD206, and MGL-1 antibodies followed by flow cytometry analysis. The results showed that the number of LPS+HKUOT-S2 polarized CD206, and MGL-1double-stained M1 macrophages (70.5% CD206+ and MGL-1+ cells) decreased compared to that of the positive control (LPS group; 81.9% CD206+ and MGL-1+cells). However, the number of HKUOT-S2-induced CD206+ cells in the polarized M1 macrophages (18.4% CD206+ cells) increased compared to that of the positive control (LPS group; 12.4% CD206+ cells) (FIG. 6F). Furthermore, IL4+HKUOT-S2 slightly increased CD206, and MGL-1double-stained M2 macrophages (92.1% CD206+ and MGL-1+ cells) compared to that of the positive control (IL4 group; 87.6% CD206+ and MGL-1+ cells) (FIG. 6F). Additionally, total protein lysates from the HKUOT-S2 polarized M1 and M2 macrophages were subjected to western blot analysis using ARG-1 antibody (M2 macrophage marker). Surprisingly, the LPS+HKUOT-S2 increased the ARG-1 protein levels in the M1 macrophage phenotype (FIG. 6G). HKUOT-S2 treatment also increased an anti-inflammatory gene, Ampka1 in the polarized M1 macrophage phenotype (FIG. 6H).

### Example 16—HKUOT-S2 Treatment Significantly Induced Cytokine Array Changes of the M1 and M2 Macrophage Condition Media (CM)

HKUOT-S2 has the potential to modulate M1 and M2 macrophage polarization (FIGS. 6B-6E). It was therefore hypothesized that HKUOT-S2 might induce secretion of some soluble factors in polarized macrophages for osteo-immunomodulatory activities. To test this hypothesis, The HKUOT-S2-induced M1 and M2 macrophage CM were subjected to cytokine array analysis. The results showed that HKUOT-S2 treatment decreased CCL17, CCL22, CXCL16, GDF-15, OPN but increased CD14 and CD54 cytokines in the M1 macrophage CM. HKUOT-S2 treatment also increased G-CSF but decreased GDF-15 cytokines in the M2 macrophage CM (FIG. 6I). These outcomes were supported by the report that an increase and decrease of these cytokines favored osteogenesis.^{[45]}

### Example 17—HKUOT-S2-Induced Macrophage CM Enhanced Osteoblast Biomineralization

Next, U937 cells (human monocyte cell line) were polarized into M1 and M2 macrophage with or without HKUOT-S2. The collected macrophage CM were used to differentiate hMSCs to osteoblasts for 10 days. Alizarin Red S staining results showed that HKUOT-S2-treated M1 macrophage CM significantly increased osteoblast biomineralization (FIGS. 6J, 6K), consistent with reports by Lu et al., ^{[23]}and Huang et al. ^{[46]}The HKUOT-S2-treated M2 macrophage CM also increased osteoblast biomineralization (FIGS. 6J, 6L).

### Example 18—HKUOT-S2 Modulates mTOR1/4E-Bp1/Akt1 Axis to Promote Osteogenesis

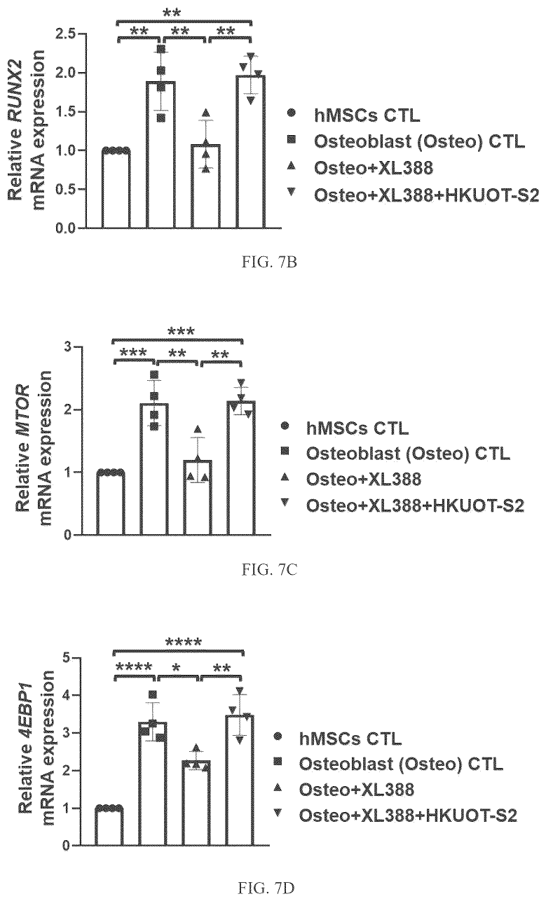

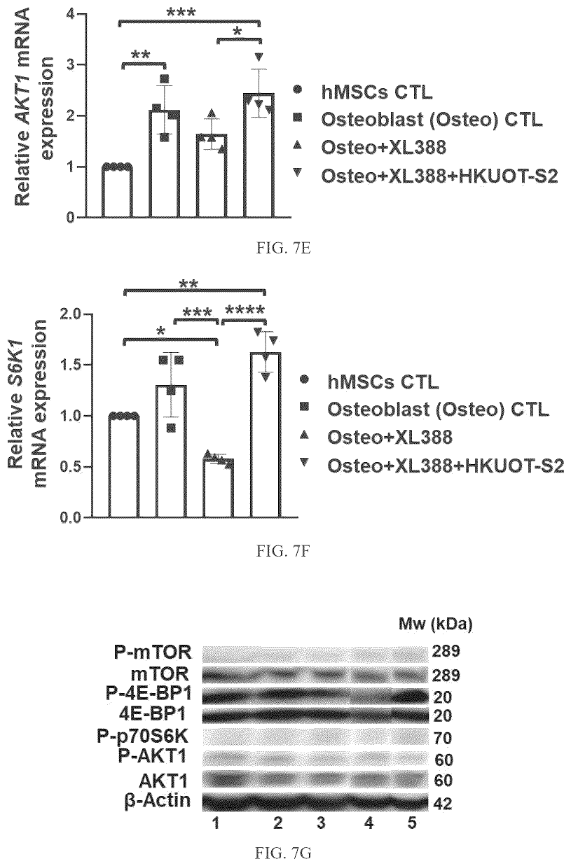

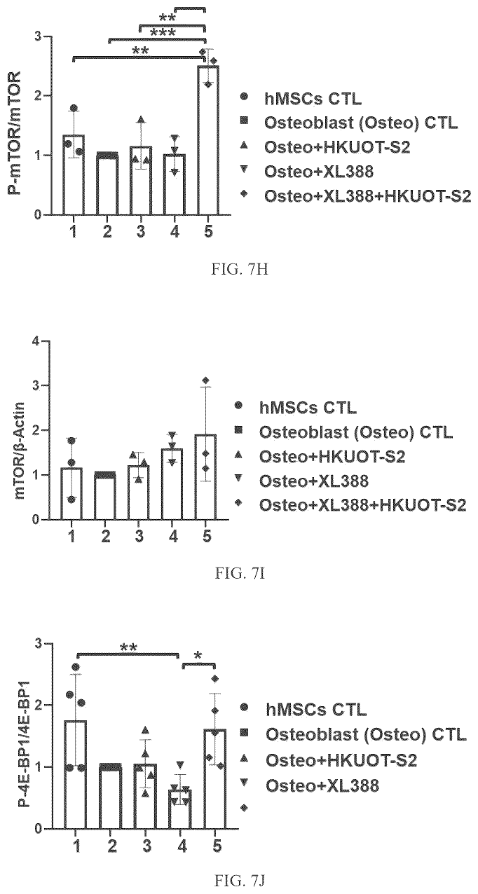

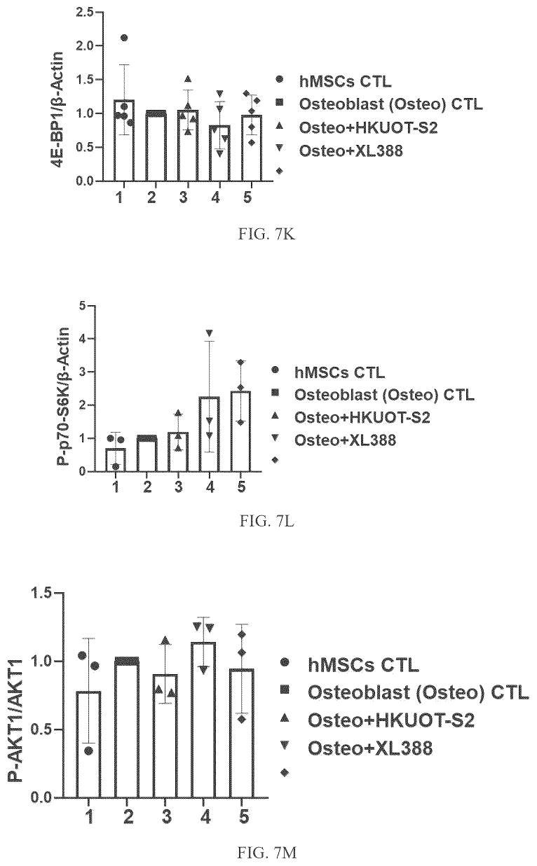

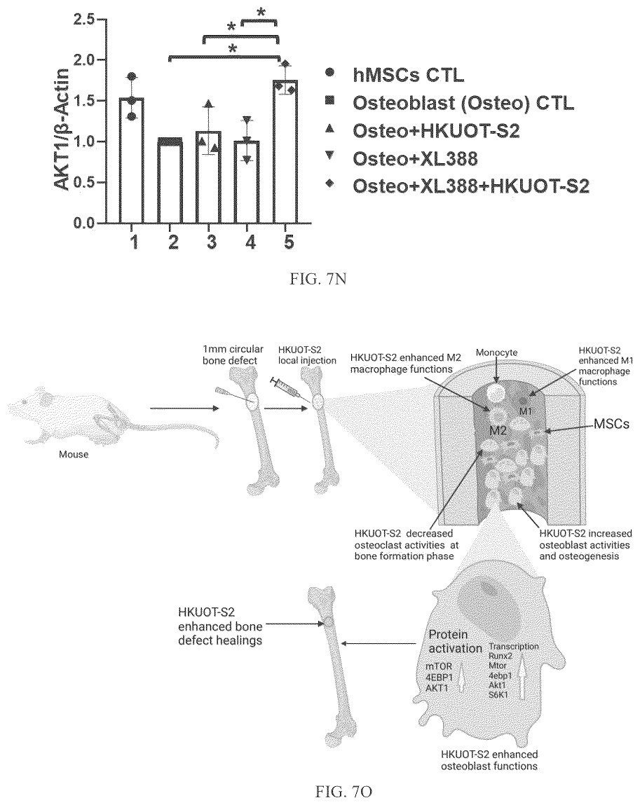

The evolutionarily conserved mammalian target of rapamycin (mTOR) signaling pathway plays pivotal roles in modulating osteogenesis and bone integrity. ^{[47, 48]}It was reported that ablation of mTOR1 signaling impaired skeletal development and growth in mice. ^{[48]}The transcriptome data revealed that HKUOT-S2-induced differentially expressed genes were significantly enriched in mTOR protein complex and signaling pathway (FIG. 4M). It was therefore hypothesized that HKUOT-S2 treatments could mechanistically modulate mTOR to enhance osteogenesis. To test this hypothesis, it was first confirmed that 0.1 gml⁻¹ HKUOT-S2 could enhance hMSCs-osteoblast differentiation in vitro (FIG. 21A). The hMSCs were then differentiated into osteoblast with/without an mTOR inhibitor (XL388) or HKUOT-S2 followed by qPCR and western blot analyses. The qPCR results showed that 125 nM XL88 treatment significantly suppressed RUNX2, MTOR1, 4E-BP1, AKT1 and S6K1 expressions (FIGS. 7A-7F). However, 0.1 μml⁻¹ HKUOT-S2 treatment blocked the XL388 inhibitory effects to increase the expressions RUNX2, MTOR1, 4E-BP1, AKT1 and S6K1 during osteoblast differentiation (FIGS. 7A-7F). Western plot analysis further confirmed that 250 nM XL88 treatments prevented the phosphorylation of MTOR1 and its downstream target 4E-BP1. HKUOT-S2 treatment blocked the XL388 inhibitory effects and increased the phosphorylation of MTOR1 and 4EBP1 proteins (FIGS. 7G, 7H, 7J). HKUOT-S2 treatment also increased total AKT1 protein level, which is another MTOR downstream target (FIG. 7N). The proteins were normalized with B-ACTIN or total proteins when necessary (FIGS. 7G-7N). It was therefore proposed in this research that HKUOT-S2 treatment could activate the MTOR1/4E-BP1/AKT1 signaling axis to promote osteogenesis (FIG. 7O).

### Example 19—HKUOT-S2 Treatment Induced Neuromodulatory Effects to Enhance Bone Defect Repairs

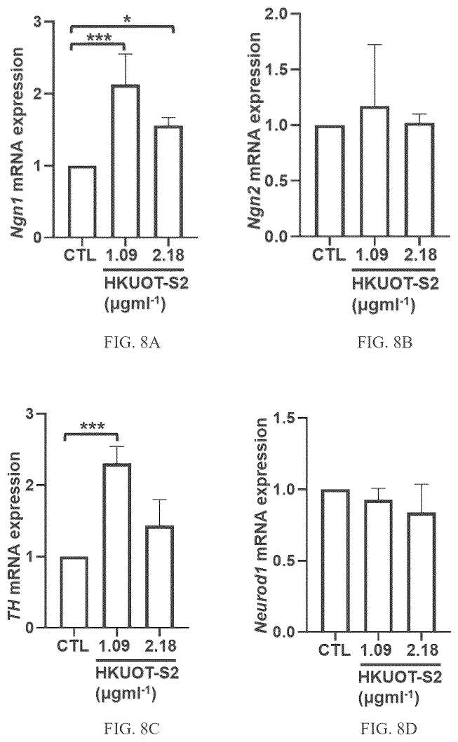

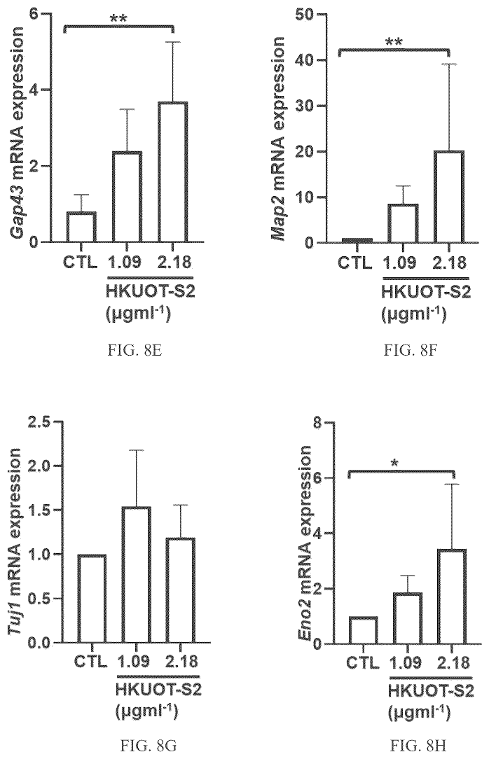

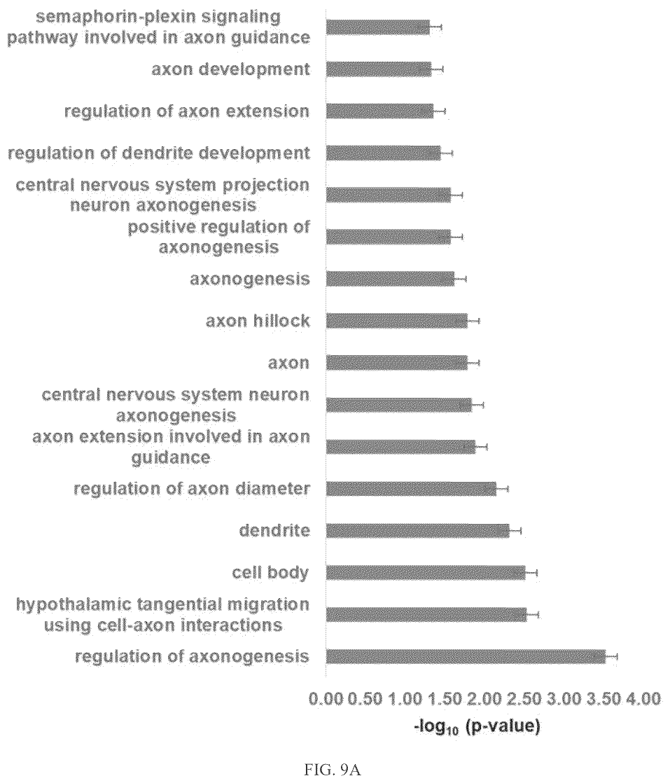

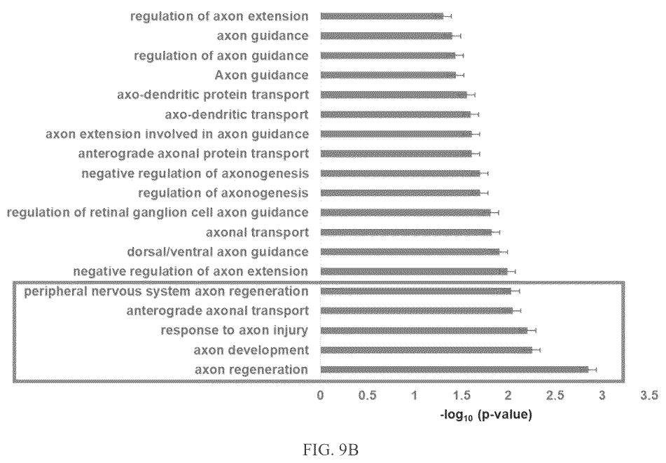

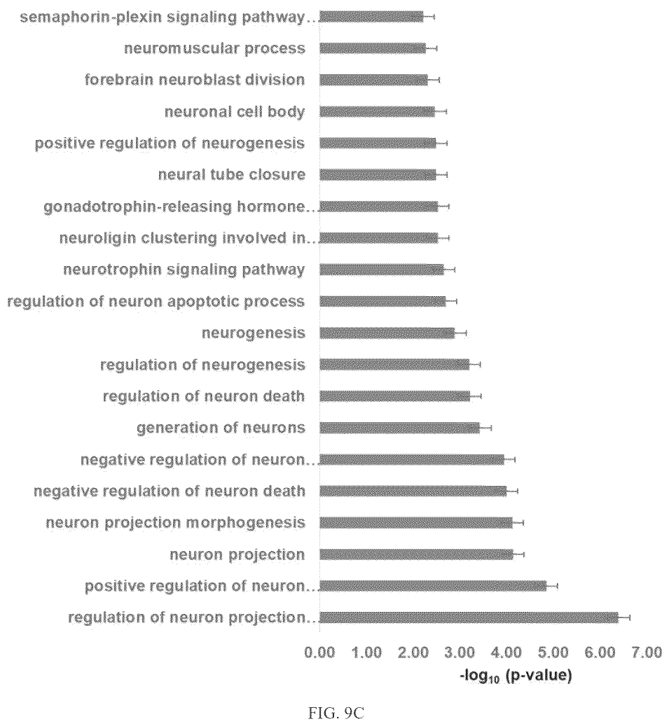

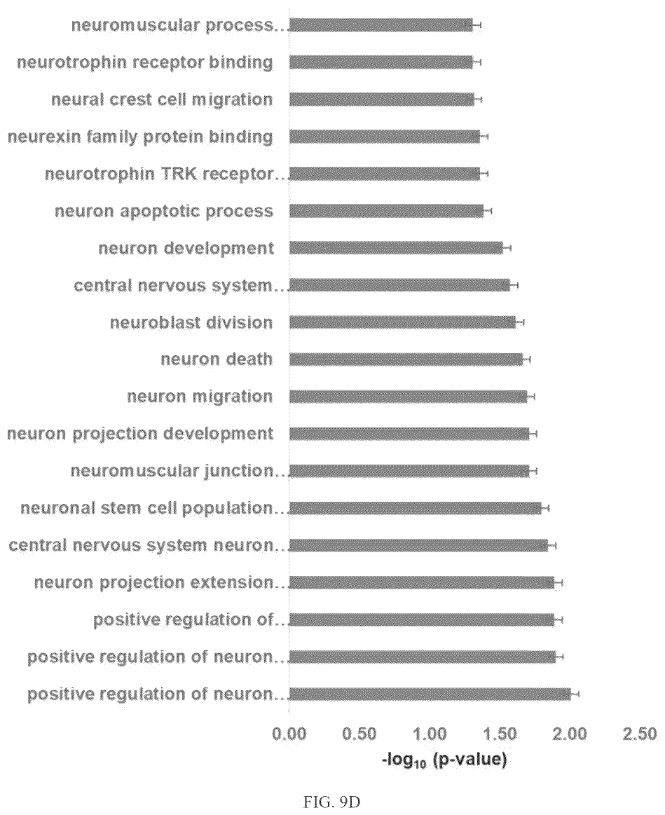

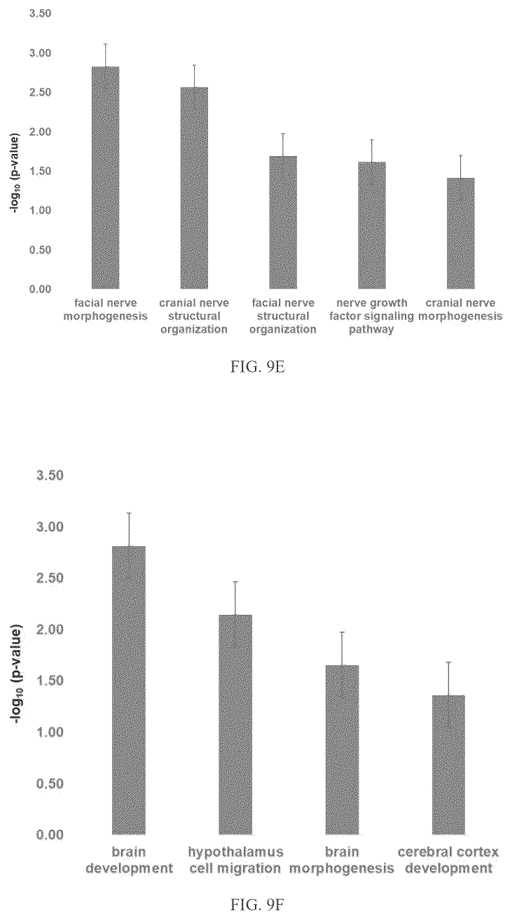

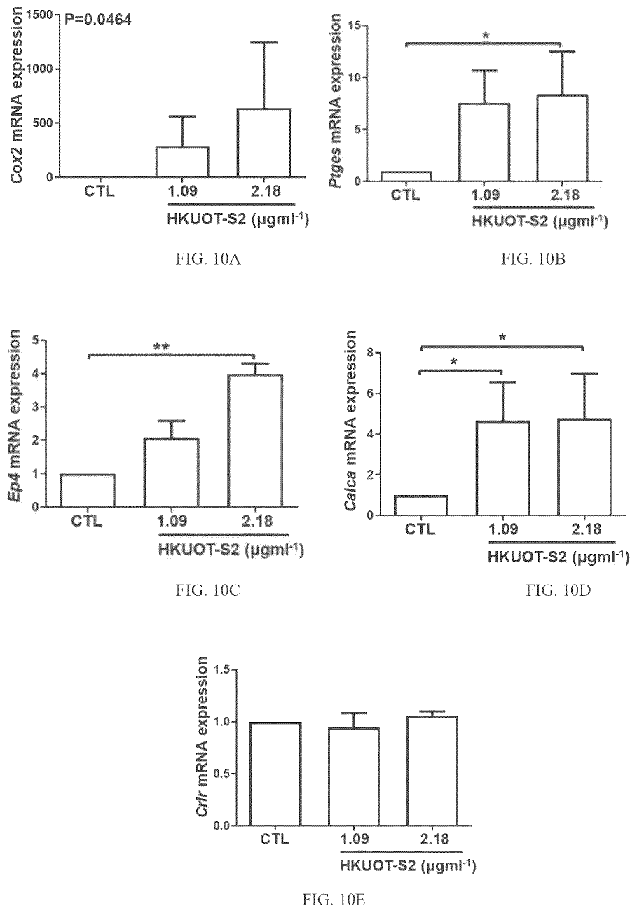

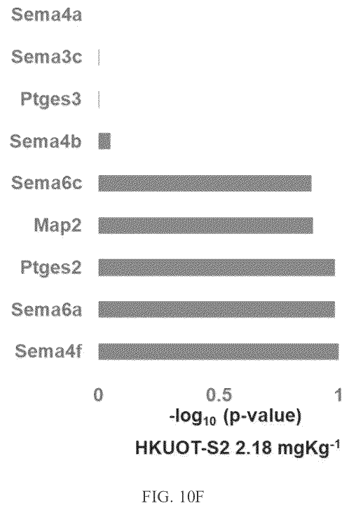

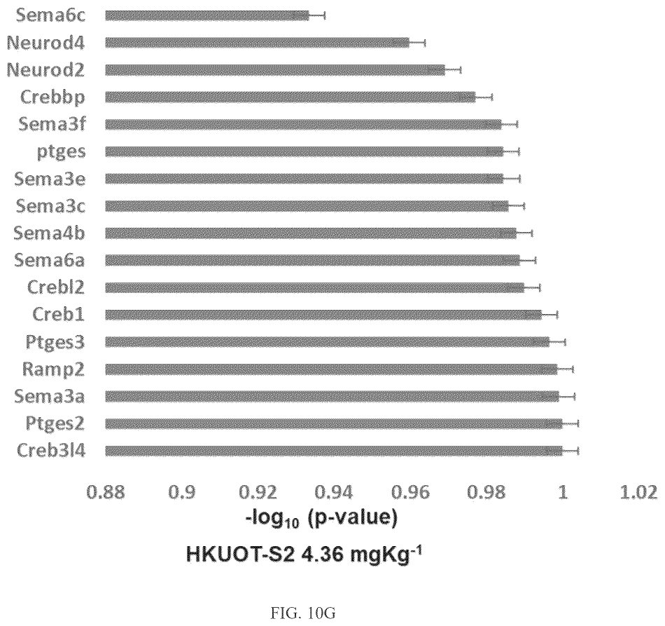

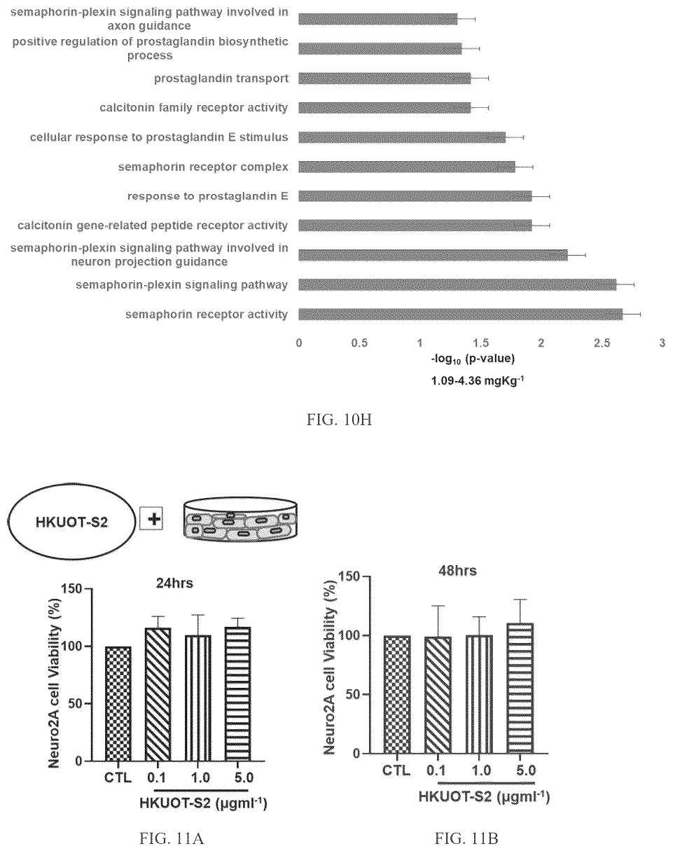

A circular hole was drilled in the mice femurs followed by HKUOT-S2 treatments for 4 weeks. The weekly micro-computed tomography (μCT) scan analysis showed that HKUOT-S2 protein promoted bone defect healing in vivo. HKUOT-S2 treatments also induced neuromodulatory effects to promote bone defect repairs in vivo (FIGS. 8A-8H, 9A-9F, 10A-10H).

### Example 20—HKOUT-S2 Treatment Promotes Neuron Differentiation

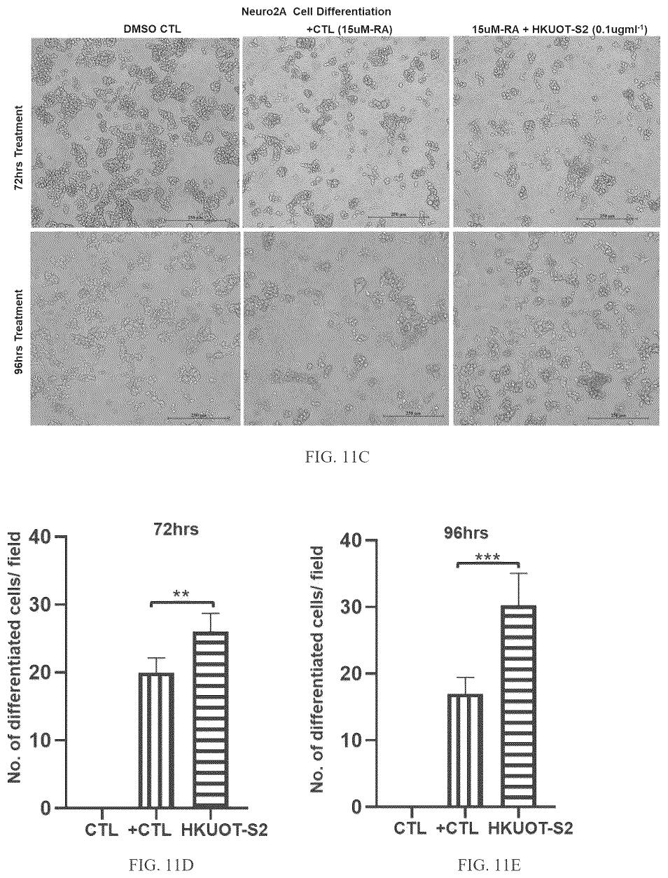

The effects of HKUOT-S2 protein on the neuroblastoma cell line, Neuro2A cells, viability was performed in vitro. The results showed that HKUOT-S2 had no significant effects on the Neuro2A cells cell viability (FIGS. 11A, 11B). Furthermore, the effects of HKUOT-S2 protein on Neuro2A cells to neuron differentiation was performed in vitro. The number of differentiated cells per field was counted for the three groups when (1) the length of axonal process >2× cell body diameter and (2) the differentiated cells (neurons) have both Axonal process and dendrites. The results showed that HKUOT-S2 protein significantly promoted Neuro2A cells to neuron differentiation in vitro (FIGS. 11C-11E).

### Example 21—HKOUT-S2 Prevents Ovariectomized (OVX)-Induced Osteoporosis

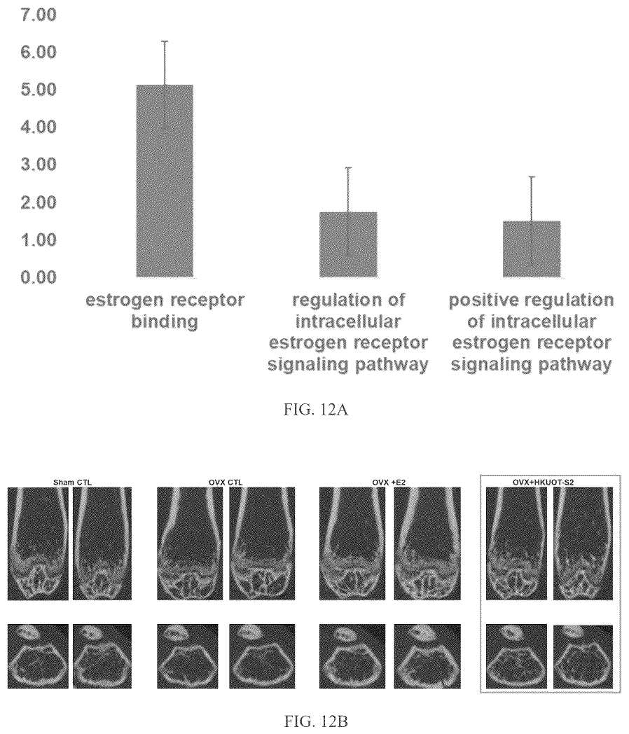

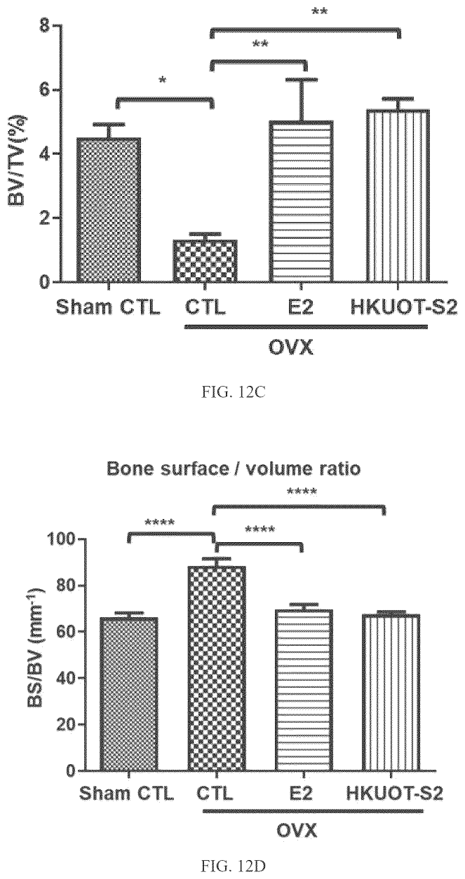

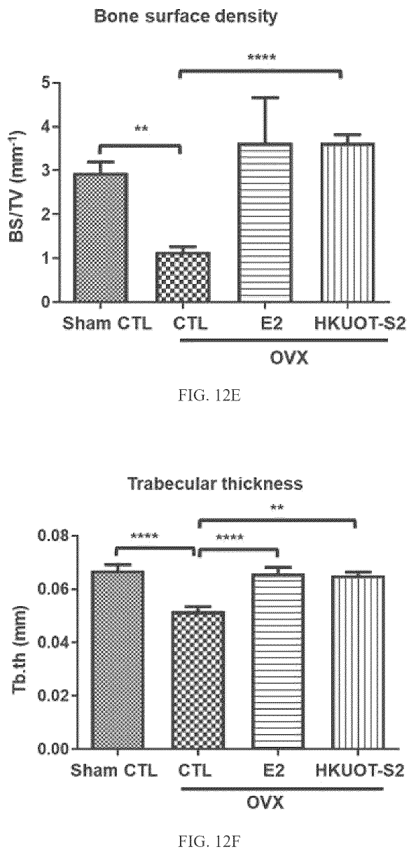

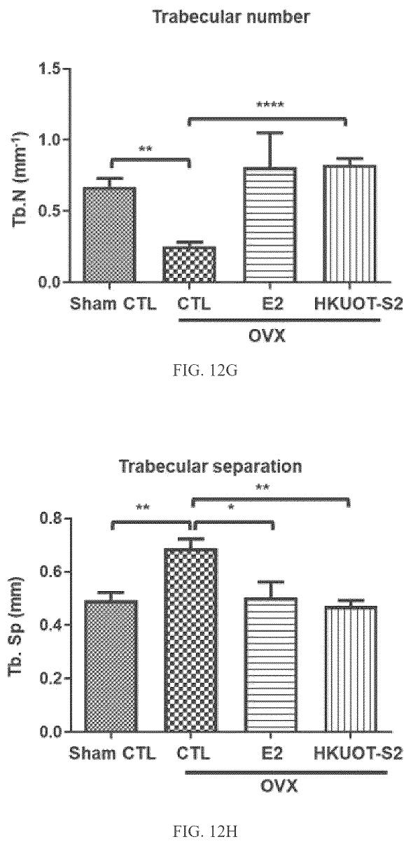

Estrogen is one of the important body hormones that promote and maintain good bone health and integrity. Ovaries are the main estrogen manufacturing factory in females. Aging in women results in gradual decline in ovarian functions. Concomitantly, estrogen levels also decline and reach the lowest levels at the menopausal stage. The decline in estrogen levels is associated with bone porosity and subsequent development of osteoporosis. An FDA-approved estrogen replacement is one of the commonly used therapeutic interventions for preventing osteoporosis. The continuous use of estrogen replacement therapy for osteoporosis, however, reportedly has drawbacks such as development of blood clotting problems and breast cancer. Finding appropriate osteogenic interventions with no or minimal side effects that could help maintain good bone health, integrity and functions during age-related estrogen declination could help prevent development of osteoporosis. It was demonstrated in our lab that the novel HKUOT-S2 protein could promote osteogenesis, and new bone formation to facilitate bone defect repairs in vivo. The safety of the therapeutic dose of HKUOT-S2 in in vivo application has been confirmed. The transcriptome data from in vivo studies also revealed that HKUOT-S2 treatments significantly enriched biological processes and functions associated with the response to estrogen receptor signaling pathways in the wild type mice (FIG. 12A). It is therefore hypothesized that HKUOT-S2 protein could modulate the estrogen receptor signaling pathways to prevent osteoporosis development in vivo. The aim of this current research is to investigate the clinical application of HKUOT-S2 protein in prevention of menopause associated osteoporosis development in ovariectomized (OVX) mice.

It has been demonstrated that OVX induced significant bone loss that is responsive to estrogen treatment in C57BL/6J mice. A total of 32 female C57BL/6J mice at the age of 8-12 weeks old and weighing between 22-25 g were used in this study. All mice underwent dorsoventral surgical operation under anesthesia. The sham control underwent surgery without ovary removal. The ovaries were completely removed from the anterior uterine horns in the OVX group. There were four groups, namely sham control (n=8), OVX control (n=8), OVX+30 μg kg⁻¹ estrogen (E2, n=8), and OVX+2.18 mg kg⁻¹ HKUOT-S2 (n=8) treatment groups. The sham and OVX control mice were given subcutaneous of 200 μl of 1XBP. The OVX+E2 and OVX+HKUOT-S2 groups were given 200 μl E2 and HKUOT-S2 via subcutaneous and IP injections respectively. All treatments started 4 hrs after the surgery and thrice per week for 4 weeks post-surgery. Micro CT (μCT) scans were taken on day zero and biweekly post-surgery. At the experimental endpoint, all the mice in the groups were sacrificed under overdose of anesthesia, the bone tissues harvested and processed for bone histomorphometry and immunohistochemical analyses.

Our preliminary results revealed that both 1.09 and 2.18 mg kg⁻¹ HKUOT-S2 treatment significant enriched biological processes and functions associated with response to estrogen receptor signaling pathways in the wild type mice (FIG. 12A). The μCT scan results showed that 2.18 mg kg⁻¹ HKUOT-S2 treatment prevented OVX-induced bone loss and osteoporosis development by increasing bone volume (BV/TV (%)), bone surface density (BS/TV), trabecular thickness (Tb. th), and trabecular number (Tb. N) but decreasing bone surface/volume ratio (BS/BV), and trabecular separation (Tb. Sp) (FIG. 12B-12H).

### Example 22—Hkuot-S2 Inhibits Progressive Glucocorticoid-Induced Osteoporosis

Glucocorticoids such as cortisone, dexamethasone and prednisolone exert anti-inflammatory and immunosuppressive activities. They are generally used as medical interventions for the treatment of pathological conditions caused by hyperactivity of the immune system. Consequently, glucocorticoids are used to treat autoimmune diseases, asthma, and severe allergies. In the early onset of the COVID-19 pandemic, dexamethasone and hydroxychloroquine cocktail was even considered as short-term treatment COVID-19 patients with severe symptoms. Many side effects of the prolonged use of glucocorticoid medications are well documented as they are generally administered in high dose to elicit the desired therapeutic effects. It is well documented that prolog use of glucocorticoids such as dexamethasone is detrimental to bone health. Glucocorticoid treatments reportedly increase bone resorption by promoting osteoclastogenesis and suppress new bone formation by decreasing osteoblastogenesis in patients. Patients under glucocorticoid therapies therefore have very high risks of osteoporosis. All patients undergoing glucocorticoid treatments are recommended to take bisphosphonates, vitamin D and calcium supplements to prevent progressive bone loss that results in drug-induced osteoporosis. However, the prolong use of bisphosphonates (anti-osteoporotic therapies) also have well documented drawbacks such as development of osteonecrosis, esophageal inflammation, severe muscle and joint pains. There are therefore high demands for medical interventions against glucocorticoid-induced osteoporosis with minimal or no side effects. Our group has discovered a novel osteogenic HKUOT-S2 protein with less safety concerns for clinical applications. It was further demonstrated HKUOT-S2 protein could enhance both inflammatory M1 and anti-inflammatory M2 macrophage polarizations, osteogenesis, and new bone formation to promote bone defect repairs in vivo. The transcriptome data from in vivo studies also revealed that HKUOT-S2 treatments significantly enriched biological processes and functions associated with the response to glucocorticoid stimuli in wild type mice (FIG. 1A). It is hypothesized in this research that HKUOT-S2 protein could enhance new bone formation by increasing osteoblastogenesis to suppress progressive glucocorticoid-induced osteoporosis development in vivo. The aim of this research is to investigate and establish the possible mechanisms by which HKUOT-S2 protein could suppress progressive glucocorticoid-induced osteoporosis development.

It was demonstrated in animal models that dexamethasone treatment could induce osteoporosis. In this research, dexamethasone (Dex) will be employed as agent for inducing osteoporosis in mice. A total of 32 male C57BL/6J mice at the age of 8-10 weeks old and weighing between 22-25 g will be used in this study. To test the research hypothesis, the mice will be subjected to dexamethasone (Dex)-induced osteoporosis. There were four groups, namely sham control (n=8), 50 mg kg⁻¹ Dex control (n=8), Dex+ 10 mg kg⁻¹ risedronate (Ris) (positive control, n=8), and Dex+ 2.18 mg kg⁻¹ HKUOT-S2 (n=8) treatment groups. The sham and Dex control mice will be given intraperitoneal (IP) injection of 200 μl of 1XBP. The Dex+ Ris and Dex+HKUOT-S2 groups will be given 200 μl Ris and HKUOT-S2 via IP injections. All treatments will be administered thrice per week for 4 weeks post-surgery. Micro CT (ρCT) scans will be taken on day zero and biweekly post-surgery. At the experimental endpoint, all the mice in the groups will be sacrificed under overdose of anesthesia, the bone tissues harvested and processed for bone histomorphometry and immunohistochemical analyses.

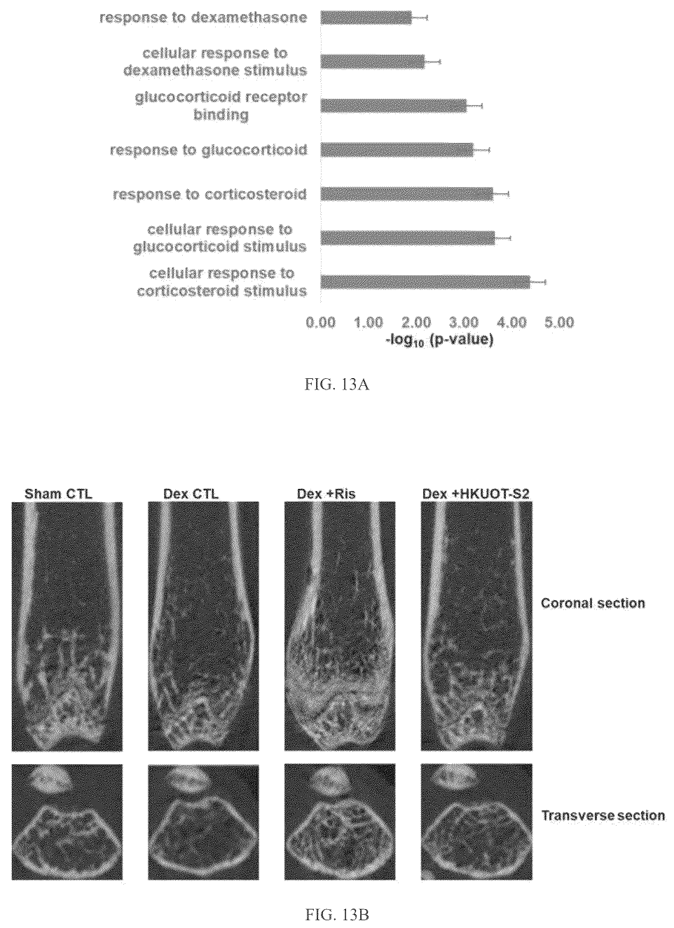

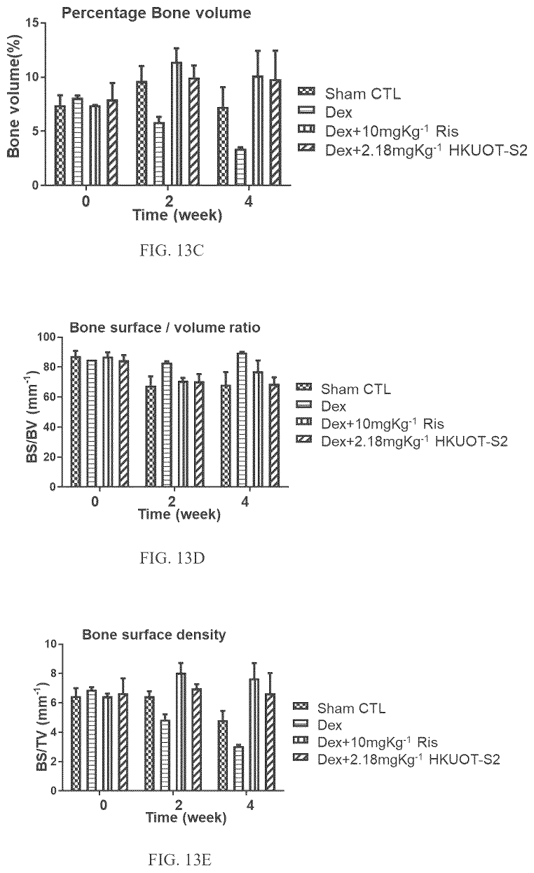

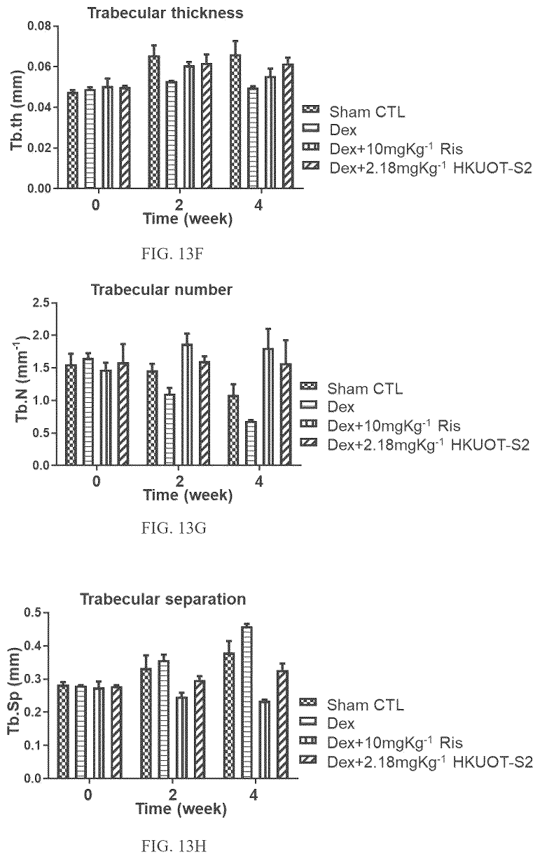

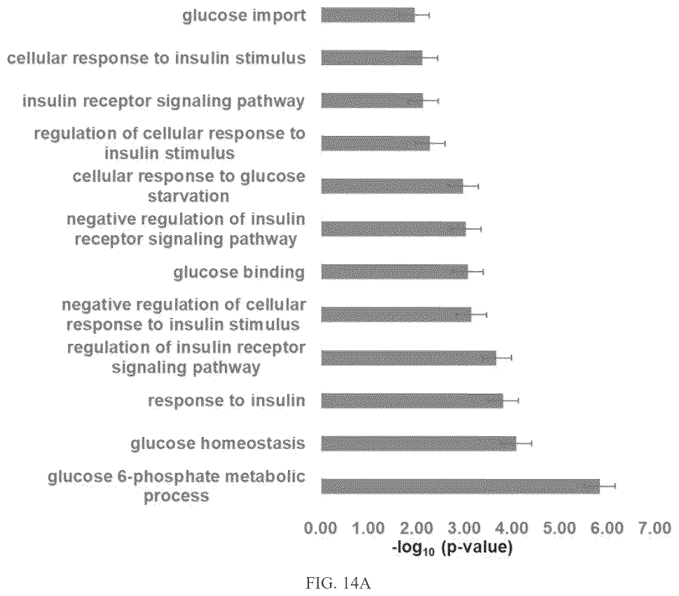

Our preliminary results revealed that both 1.09 and 2.18 mg kg⁻¹ HKUOT-S2 significant enriched biological processes and functions associated with the response to glucocorticoid stimuli including dexamethasone (Dex) in the wild type mice (FIG. 13A). In the Dex-induced osteoporotic model, the preliminary results showed that 2.18 mg kg⁻¹ HKUOT-S2 could suppress the Dex-induced osteoporosis development by increasing the percentage bone volume (BV/TV), bon surface density (BS/TV), Tb. th and trabecular number (Tb. N) (FIGS. 13B, 13C, 13E-13G) but decreasing bone surface/volume ratio (BS/BV) and trabecular separation (Tb. Sp) (FIGS. 13D, 13H) just as that of the positive control.

### Example 23—Investigation of Antidiabetogenic and Anti-Diabetic Hkuot-S2

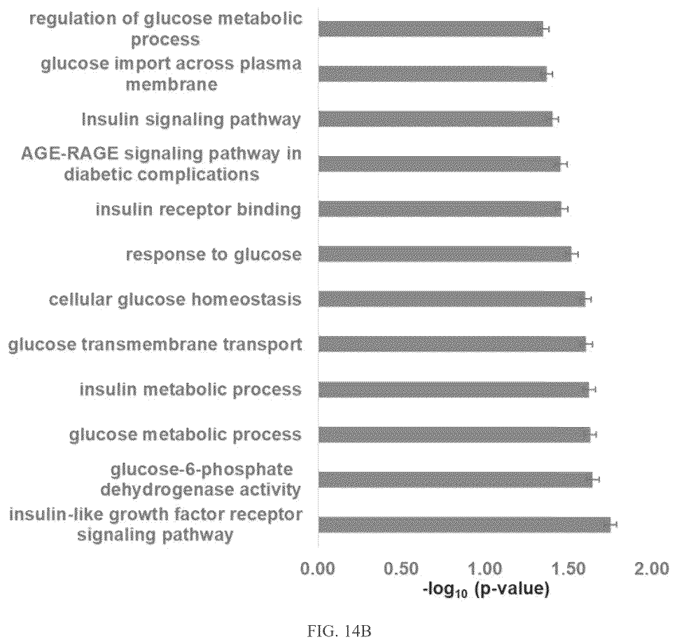

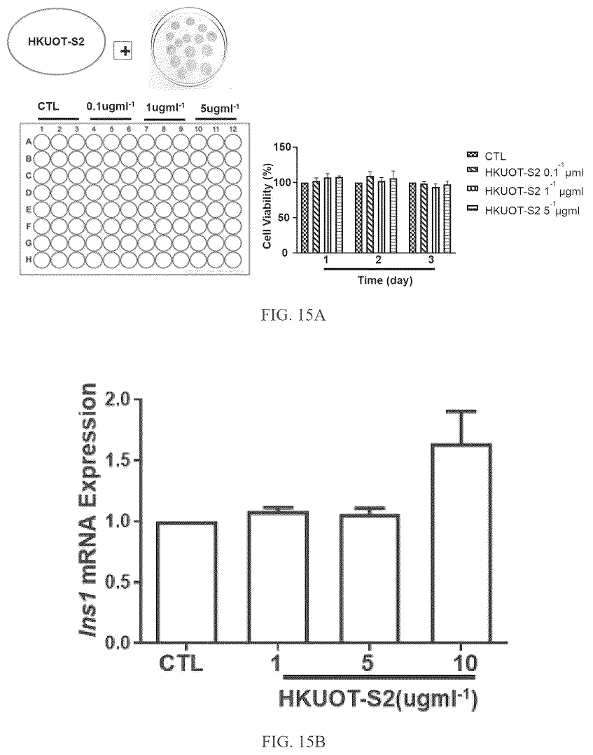

Diabetes is a multifactorial and chronic metabolic disorder usually characterized by hyperglycemia. The World Health Organization (WHO) has estimated the global number of diabetic patients to be 422 million and ranked as the 9^{th} cause of global deaths in 2019. About 1.5 million diabetic patients die per annum. The major form of diabetes is type 2 diabetes caused by insulin resistance. Type 1 diabetes is an autoimmune disorder that results in insufficient insulin production by the pancreatic β-cells. Gestational diabetes causes elevated blood sugar levels in pregnant women. Impaired glucose tolerance (IGT) and impaired fasting glycaemia (IFG) mediates between normoglycemic and hyperglycemic conditions. Pharmacological treatment of diabetes targets glucose metabolism and/or insulin production and functions to skew hyperglycemic conditions towards normoglycemia. It has been reported Dioscorea spp extracts exerts anti-diabetic property. However, the bioactive anti-diabetic ingredients and the underlying mechanism of the Dioscorea spp. extracts have not been fully explored. In our ongoing exploration of the clinical application of the novel HKUOT-S2 protein, it was discovered that HKUOT-S2 treatment significantly enriched several signaling pathways, biological processes and functions associated with glucose metabolism and insulin functions in wild type male mice (FIGS. 14A, 14B). It is therefore hypothesized that HKUOT-S2 could modulate glucose metabolism and insulin functions to suppress onset of diabetes development in mice.

### Type I Diabetes Mellitus (T1DM) Animal Model

### Multiple Low Dose Treatment of STZ

Multiple low dose STZ treatments induce inflammation actions that partially destroy the pancreatic islets and β-cells, impair insulin production and cause hyperglycemia which are pathologic resemblance phenotypes of T1DM. In this model, the C57BL/6 male mice will be given 40 mg/kg streptozotocin (STZ) in citrate buffer for five days. There shall be 4 groups namely sham control (citrate buffer, n=15), STZ CTL(n=15), STZ+ insulin (n=15) and STZ+ HKUOT-S2 groups (n=15). All injections shall be via IP route. The mice will be provided normal food and 10% sucrose water for the first five days. Thereafter, the sucrose water will be replaced by normal water. On experimental days 7, 14, 21 and 28, 2 g kg⁻¹ glucose will be given to 6 hrs fasted mice. Blood glucose levels will be measured before and after fastening using glucometer. Blood glucose levels will also be measured at 15-, 30-, 60-, and 120-minutes post-challenge glucose challenge. Blood insulin levels will also be measured. Histological, immunohistochemistry, and electron microscopic techniques as well as downstream mechanistic studies will be employed to establish the anti-diabetic and antidiabetogenic properties of the novel HKUOT-S2 protein.

### Single High Dose Treatment of STZ

Single high dose STZ treatment destroys the pancreatic β-cells resulting in T1DM phenotype. In this model, the C57BL/6 male mice will be given 200 mg/kg streptozotocin (STZ) in citrate buffer. The experimental groups, number of mice per group, and evaluation procedures will be the same as that of the multiple low dose STZ treatment experiment.

### Type II Diabetes Mellitus (T2DM) Animal Model

### Insulin-Deficient Model

Nicotinamide (NA) administration in conjunction with the with SZT protect the mice against STZ-induced diabetogenic effects by partially protecting the pancreatic β-cells. The pancreatic β-cells under the influence of STZ and NA treatments produce insufficient insulin leading to T2DM. In this model, the C57BL/6 male mice will be fasted for 6-8 hrs prior to the STZ and NA treatments. The mice will be first given IP injection of 210 mg/kg NA. Fifteen minutes after NA injection, the mice will be given 180 mg/kg STZ via IP injection. The experimental groups, number of mice per group, and evaluation procedures will be the same as that of the T1DM experiments.

### Insulin Resistance Model

Feeding mice with high-fat food followed by moderate dose STZ treatment reportedly caused hyperglycemia, hyperinsulinemia and insulin resistance. In this model, the C57BL/6 male mice will be fed high-fat diet for 21 days. On day 22, mice will be fasted for 6-8 hrs followed by 40 mg/kg STZ IP injection. Ten days post-STZ treatment, insulin resistance will be assessed in the mice. The experimental groups, number of mice per group, and evaluation procedures will be the same as that of the Insulin-deficient model. Ethical approval and animal license will be obtained to start this project.

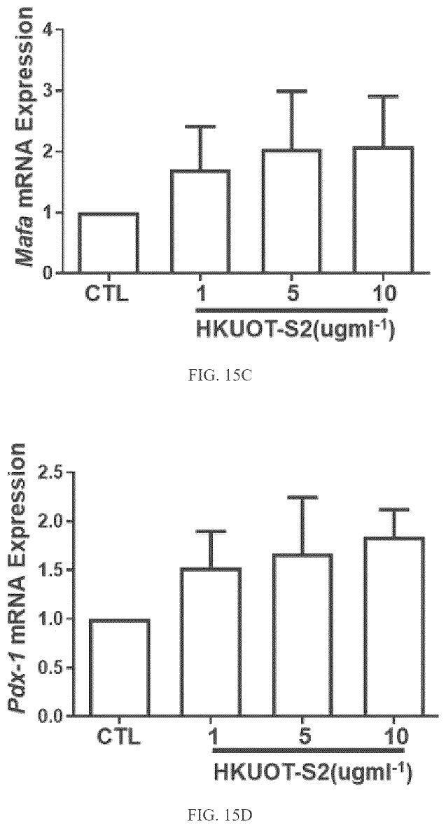

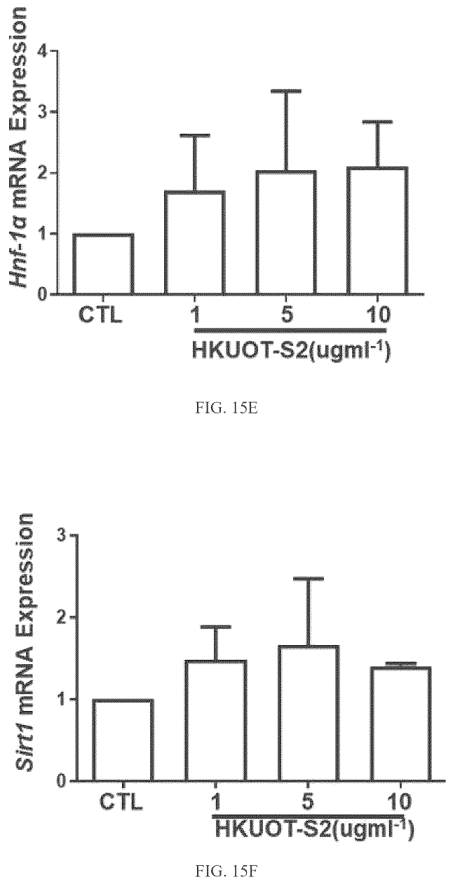

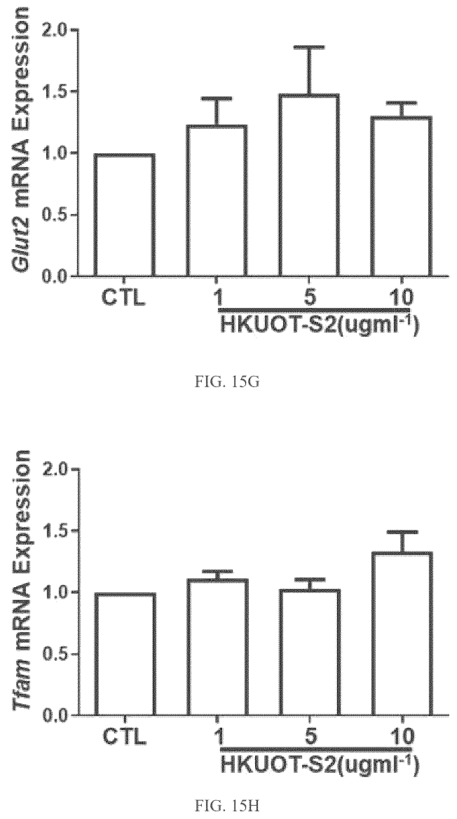

Results showed that HKUOT-S2 treatments significantly enriched glucose metabolism and insulin functional signaling pathways, biological processes and functions associated in wild type male mice (FIGS. 14A, 14B). In vitro results showed that HKUOT-S2 treatments had no effects on INS-1E cells (insulinoma cells) viability (FIG. 15A). HKUOT-S2 treatments, however, apparently increased pancreatic β-cells function genes such as Ins-1, Mafa, Pdx-1, and Hnf-1a in INS-1E cells (FIG. 15B-15H).

### Example 24—Investigation of Hkuot-S2 Protein in Male and Female Reproduction and Embryonic Development

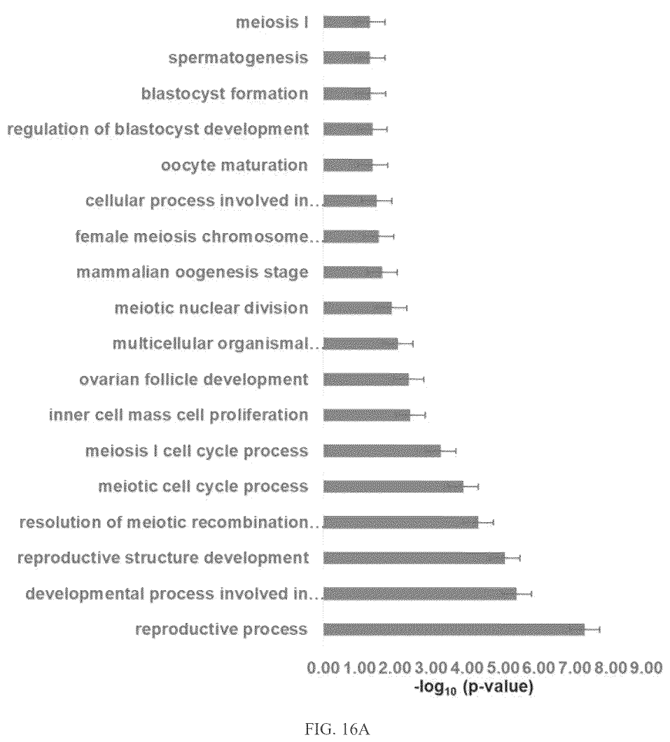

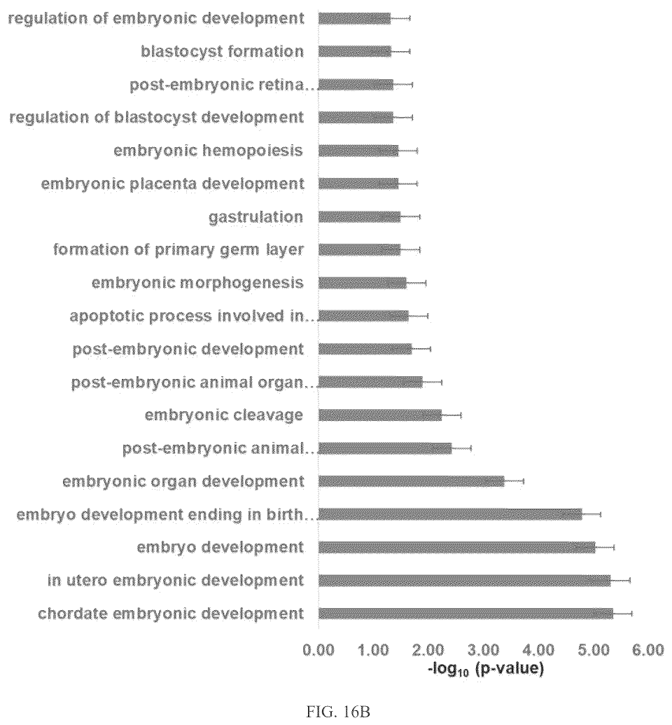

The transcriptomic data generated from the rRNA-depleted RNA-sequencing of the bone tissues from mice subjected to HKUOT-S2 treatment has been highly reliable in all our research directions. The transcriptome data from in vivo studies also revealed that HKUOT-S2 treatments significantly enriched biological processes and functions associated with oogenesis, spermatogenesis, reproductive processes and embryonic development in the wild type mice (FIGS. 16A, 16B). HKUOT-S2 protein could promote reproduction and embryonic development in vivo. HKUOT-S2 treatment apparently modulates estrogen receptor signaling and suppresses progression of OVX-induced osteoporosis in a menopausal syndrome animal model. HKUOT-S2 could be a potential intervention against some fertility problems.

The transcriptome data from in vivo studies revealed that HKUOT-S2 treatments significantly enriched biological processes and functions associated with oogenesis, spermatogenesis, reproductive processes and embryonic development in the wild type mice (FIGS. 16A, 16B).

### Example 25—Hkuot-S2-Derived Peptide Robustly Suppressed Osteoporosis Development

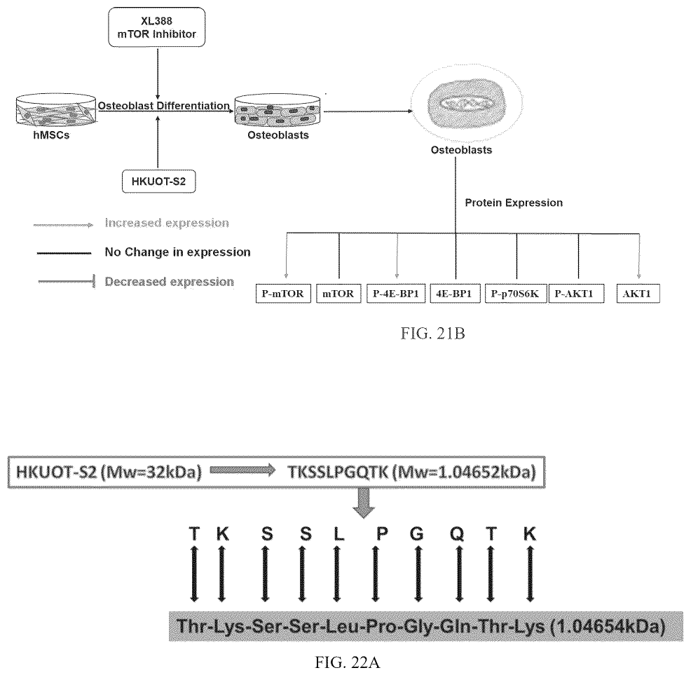

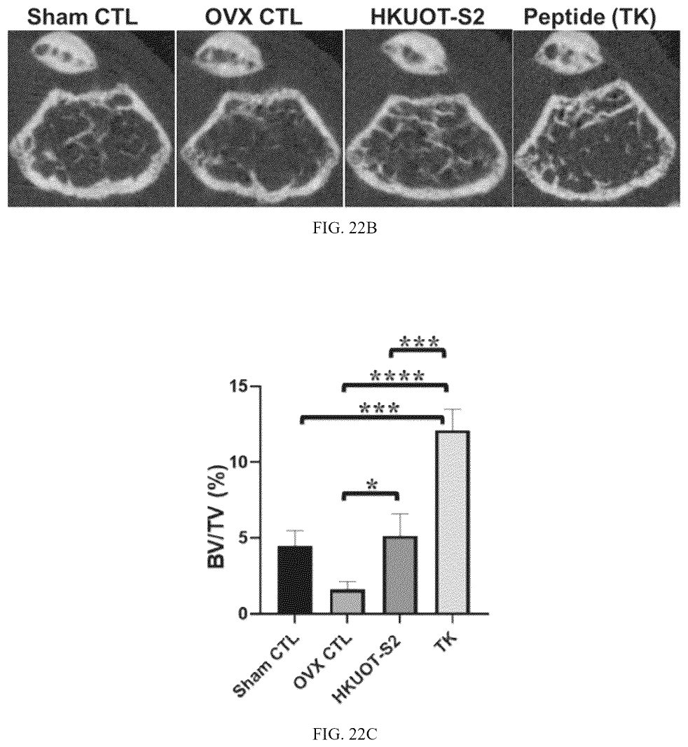

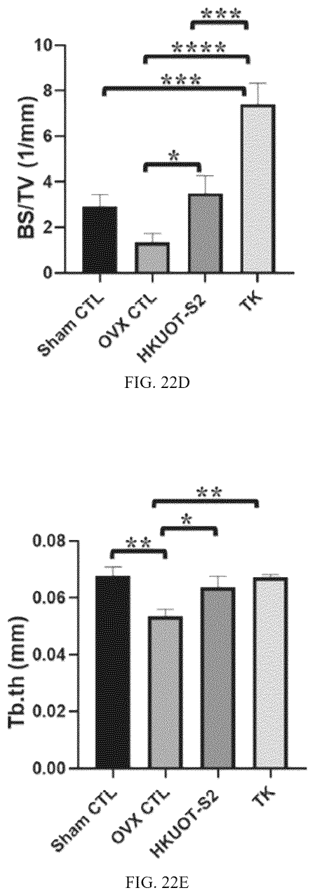

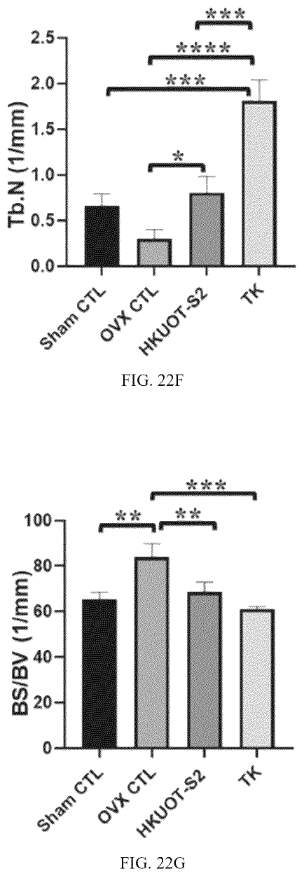

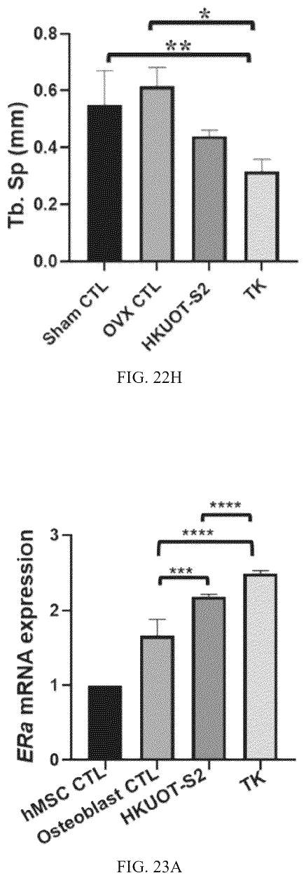

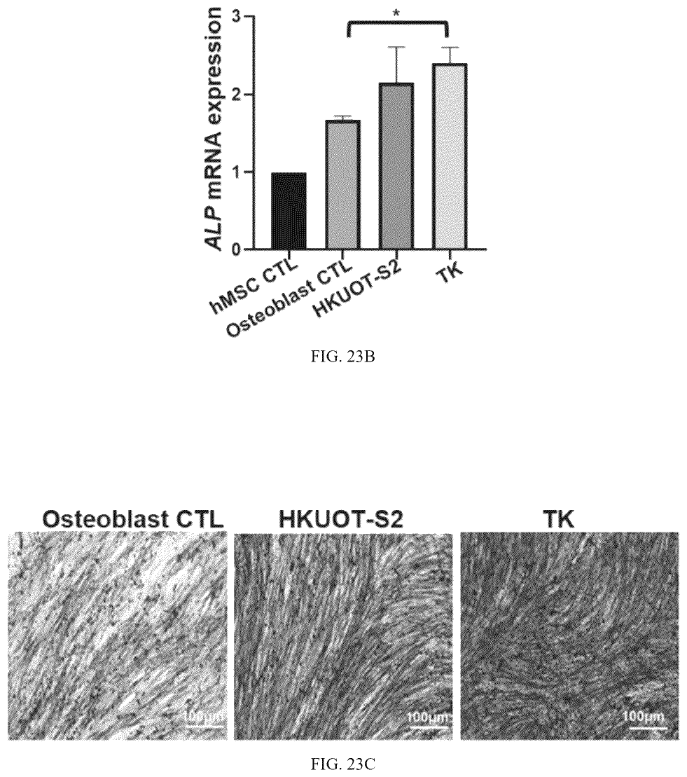

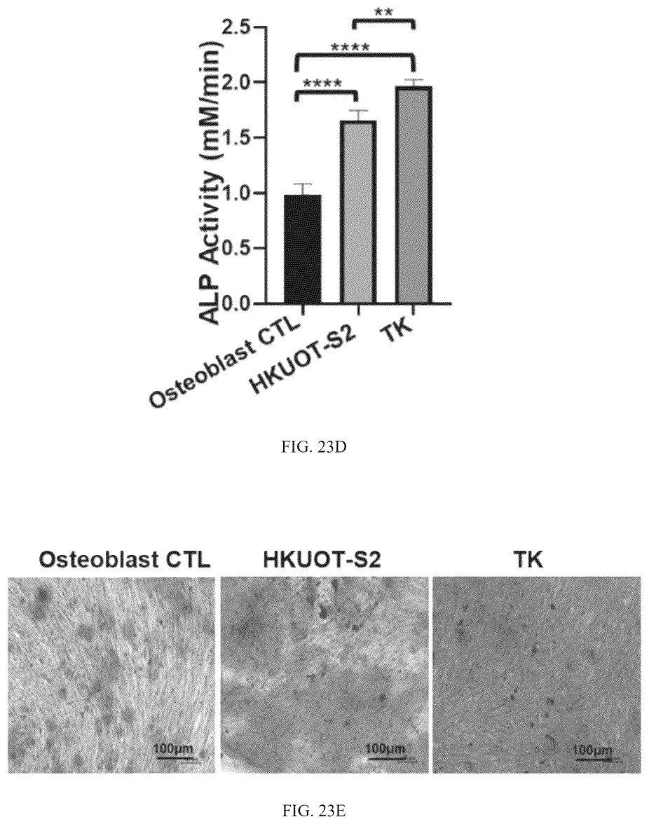

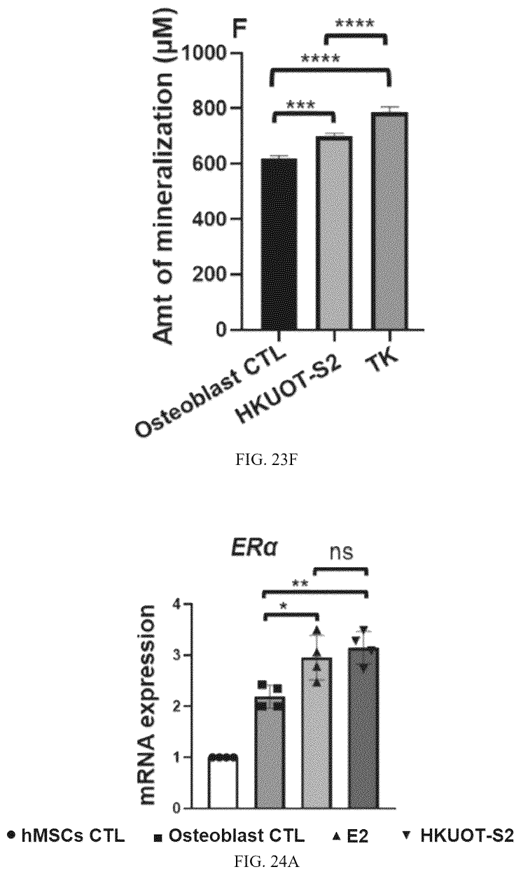

We have demonstrated that HKUOT-S2 or SEQ ID NO: 83 HKUOT-S2 peptide: TKSSLPGQTK, herein referred to as TK, can prevent the development of osteoporosis in the femur of the ovariectomized (OVX) mice. Micro-computed tomography (μCT) scan analysis showed that HKUOT-S2 protein or its derived SEQ ID NO: 83 treatments suppressed osteoporosis progression by significantly increasing bone volume (BV/TV), bone surface area to tissue volume (BS/TV) ratio, trabecular thickness (Tb.th), trabecular number (Tb. N) but decreased bone surface area to bone volume ratio (BS/BV) and trabecular separation (Tb. Sp) (FIGS. 22A-22G). It must be noted that 2.18 mg/Kg HKUOT-S2 and 0.5 mg/Kg SEQ ID NO: 83 treatments are the optimal doses that prevented osteoporosis development. Comparatively, 0.5 mg/Kg SEQ ID NO: 83 treatment possessed stronger osteoporosis suppression effects than the 2.18 mg/Kg HKUOT-S2 treatment group. Furthermore, in vitro studies showed that 0.1 μg/ml HKUOT-S2 and 0.01 μg/ml SEQ ID NO: 83 treatments promote human mesenchymal stem cells (hMSCs) to osteoblast differentiation by increasing oestrogen receptor α (ERα) and ALP mRNA expressions (FIGS. 23A-23B). The results also showed that 0.1 μg/ml HKUOT-S2 and 0.01 μg/ml SEQ ID NO: 83 treatments significantly increased ALP activities and osteoblast biomineralization in vitro (FIGS. 23C-23F). Compared to the HKUOT-S2 treatment, the SEQ ID NO: 83 has robust osteogenic effects. The osteogenic effects of the other HKUOT-S2-derived peptides such as SEQ ID NOs: 82 and 85 are under investigation.

### Example 26—Hkuot-S2 Modulates Estrogen Receptors to Promote Osteogenesis and Inhibit Osteoporosis

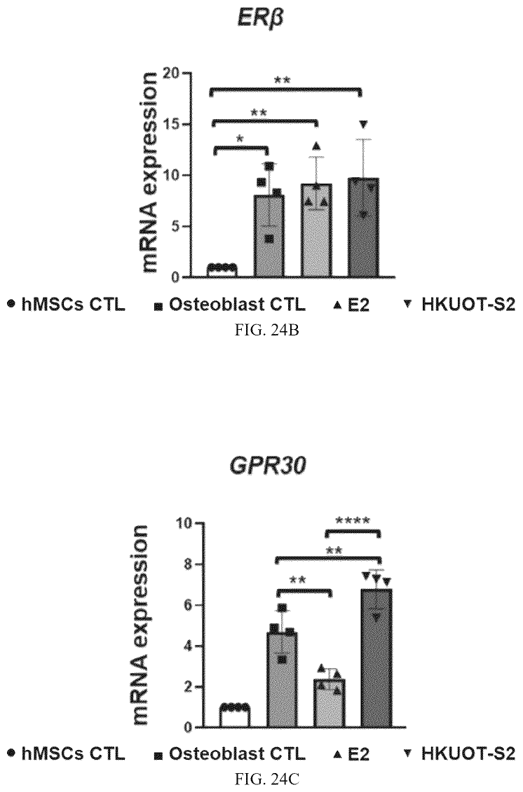

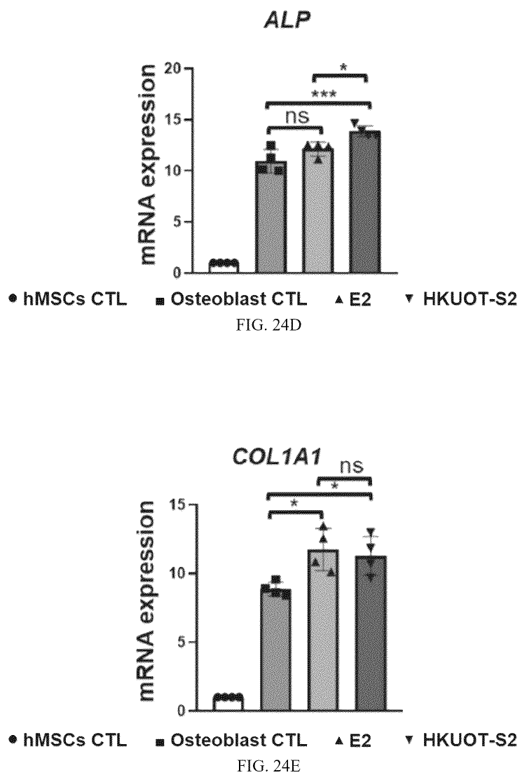

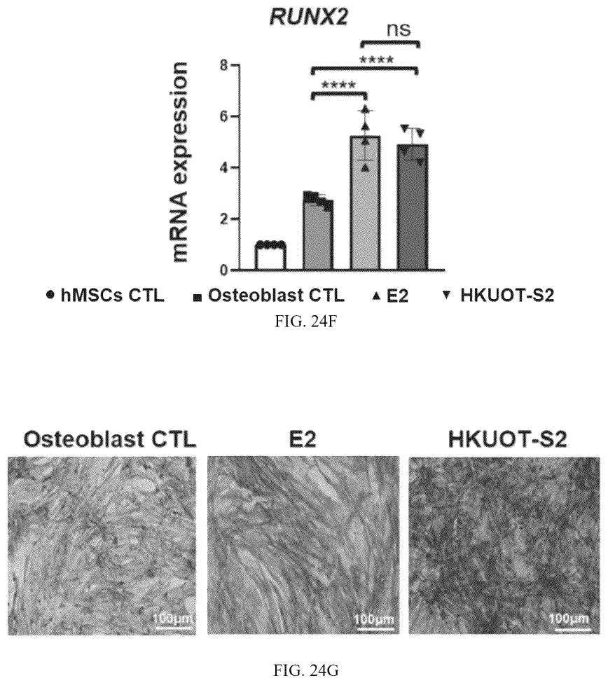

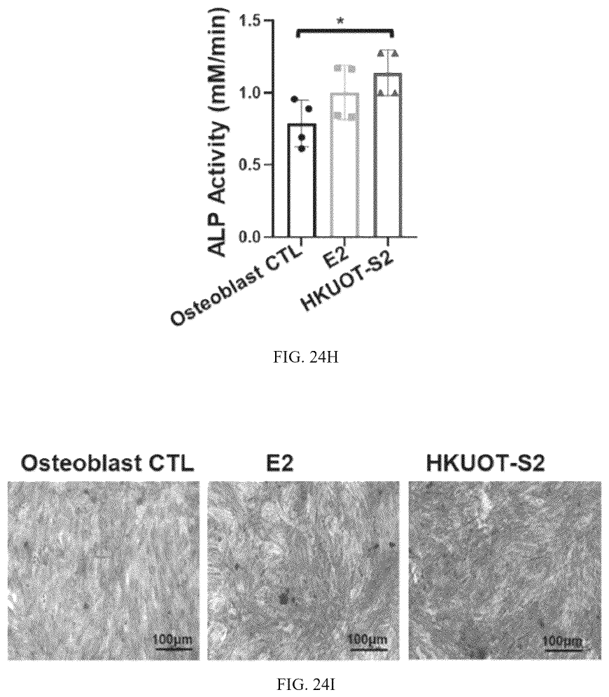

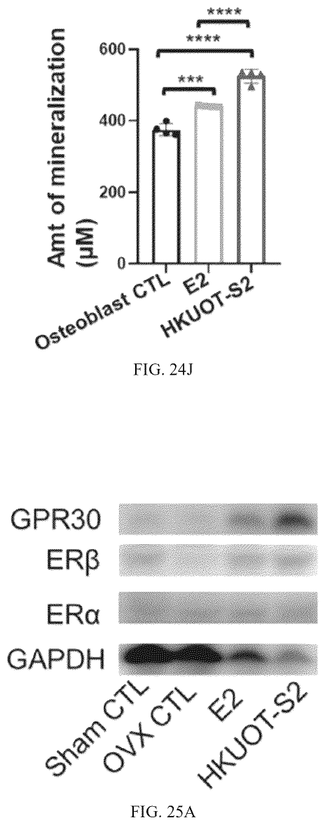

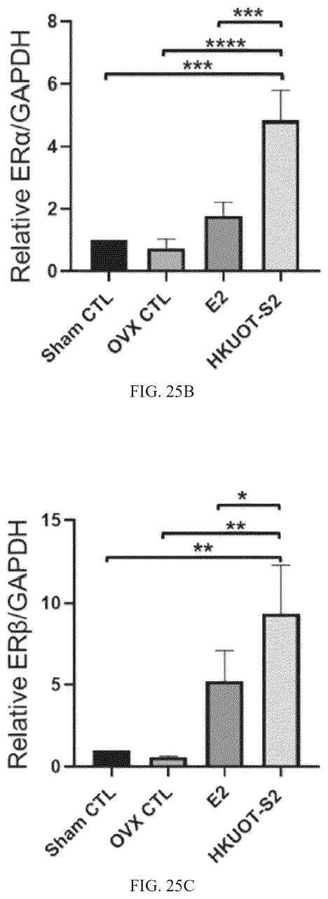

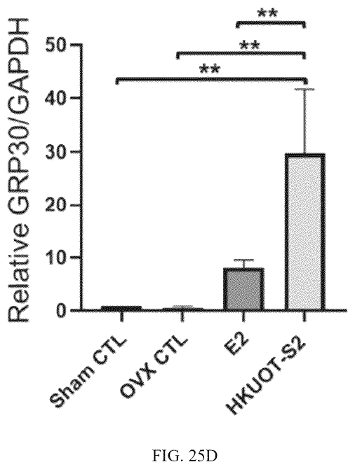

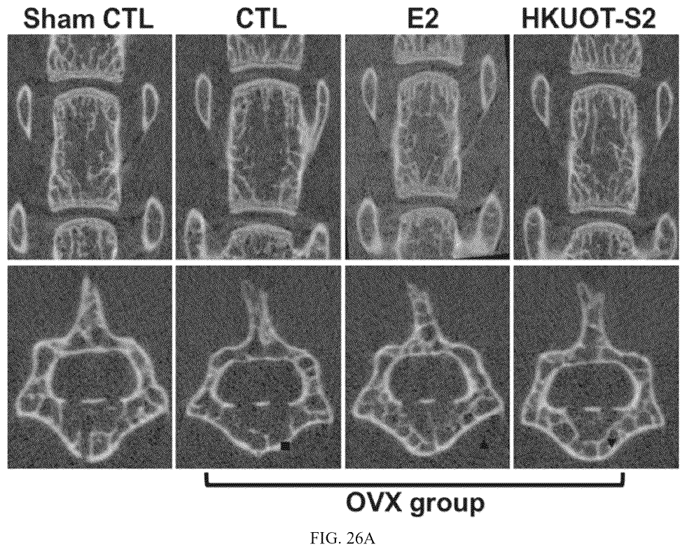

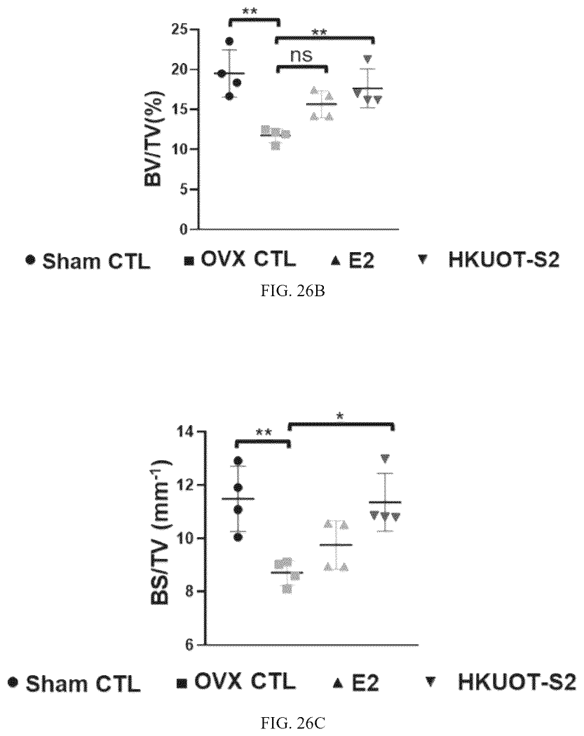

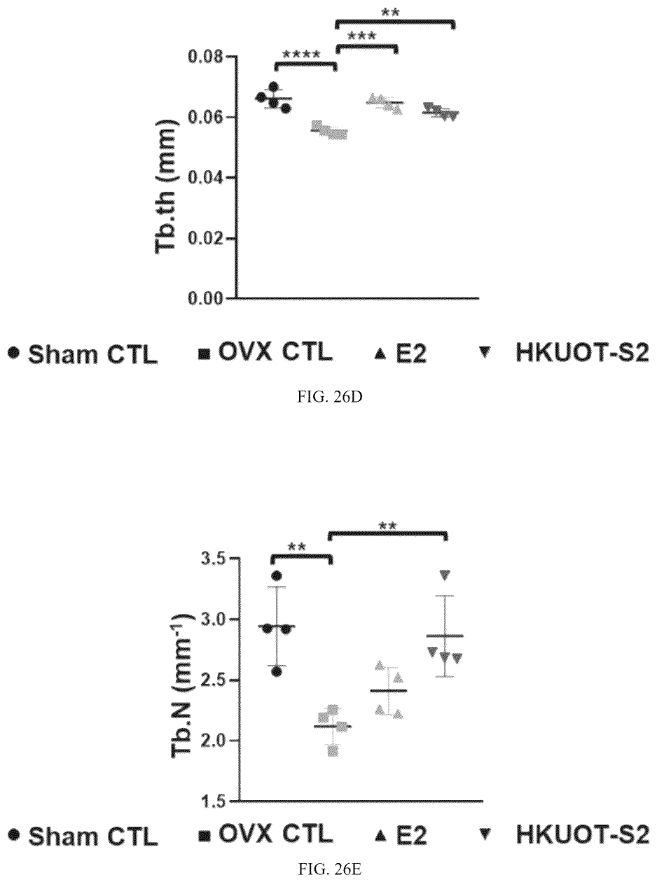

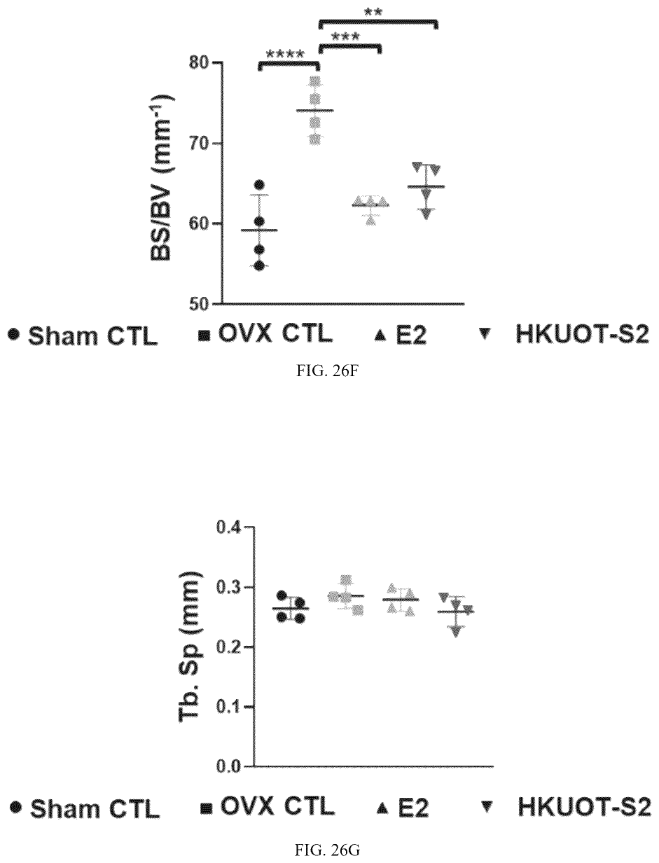

In an in vitro study, hMSCs were differentiated into osteoblast with or without estrogen (E2, positive control) or HKUOT-S2. The results showed that HKUOT-S2 treatment promoted osteoblast differentiation by increasing the expression of oestrogen receptors Era and GPR30 thereby upregulating osteogenic gene expressions of ALP, COL1A1 and RUNX2 (FIGS. 24A-24F). HKUOT-S2 treatment also significantly enhanced ALP activities and osteoblast biomineralization (FIGS. 24G-24J). In an osteoporotic model, OVX mice were treated with E2 or HKUOT-S2 protein. Western blot analysis showed that HKUOT-S2 treatment significantly increased Era, Erβ and GPR30 protein levels in bone tissue when compared with the sham control, OVX control or the E2 treatment groups (FIGS. 25A-25D). Micro-computed tomography (μCT) scan analysis of the lumbar vertebrae (L5) showed that HKUOT-S2 protein treatment suppressed osteoporosis by significantly increasing bone volume (BV/TV), bone surface area to tissue volume (BS/TV) ratio, trabecular thickness (Tb.th), trabecular number (Tb. N) but decreased bone surface area to bone volume ratio (BS/BV) with no effects on trabecular separation (Tb. Sp) (FIGS. 26A-26G). Generally, there is no significant difference between E2 and HKUOT-S2 protein treatment groups with respect to the suppression of osteoporosis development.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

SEQUENCES

SEQ ID NO: 1: Alp primer forward: 5′-CCAGCAGGTTTCTCTCTTGG-3′
SEQ ID NO: 2: Alp primer reverse: 5′-GGGATGGAGGAGAGAAGGTC-3′
SEQ ID NO: 3: Col primer forward: 5′-GAGCGGAGAGTACTGGATCG-3′
SEQ ID NO: 4: Col primer reverse: 5′-GTTCGGGCTGATGTACCAGT-3′
SEQ ID NO: 5: Opn primer forward: 5′-TCTGATGAGACCGTCACTGC-3′
SEQ ID NO: 6: Opn primer reverse: 5′-AGGTCCTCATCTGTGGCATC-3′
SEQ ID NO: 7: Runx2 primer forward: 5′-CCCAGCCACCTTTACCTACA-3′
SEQ ID NO: 8: Runx2 primer reverse: 5′-TATGGAGTGCTGCTGGTCTG-3′
SEQ ID NO: 9: Arg-1 primer forward: 5′-CTCCAAGCCAAAGTCCTTAGAG-3′
SEQ ID NO: 10: Arg-1 primer reverse: 5′-AGGAGCTGTCATTAGGGACATC-3′
SEQ ID NO: 11: Mgl-1 primer forward: 5′-TGAGAAAGGCTTTAAGAACTGGG-3′
SEQ ID NO: 12: Mgl-1 primer reverse: 5′-GACCACCTGTAGTGATGTGGG-3′
SEQ ID NO: 13: Cd206 primer forward: 5′-TTCCATCGAGACTGCTGCT-3′
SEQ ID NO: 14: Cd206 primer reverse: 5′-CCAGAGGGATCGCCTGTTT-3′
SEQ ID NO: 15: Ym1 primer forward: 5′-CATGAGCAAGACTTGCGTGAC-3′
SEQ ID NO: 16: Ym1 primer reverse: 5′-GGTCCAAACTTCCATCCTCCA-3′
SEQ ID NO: 17: Socs3 primer forward: 5′-GATTTCGCTTCGGGACTAG-3′
SEQ ID NO: 18: Socs3 primer reverse: 5′-CGGCGGCGGGAAACTTGCTG-3′
SEQ ID NO: 19: Tnfα primer forward:
5′-TCTCAGCCTCTTCTCATTCCTGC-3′
SEQ ID NO: 20: Tnfα primer reverse:
5′-AGAACTGATGAGAGGGAGGCCAT-3′
SEQ ID NO: 21: iNOS primer forward: 5′-AATCTTGGAGCGAGTTGTGG-3′
SEQ ID NO: 22: iNOS primer reverse: 5′-CAGGAAGTAGGTGAGGGCTTG-3′
SEQ ID NO: 23: IL-6 primer forward:
5′-ACAAAGCCAGAGTCCTTCAGAGAG-3′
SEQ ID NO: 24: IL-6 primer reverse:
5′-TTGGATGGTCTTGGTCCTTAGCCA-3′
SEQ ID NO: 25: IL-1β primer forward: 5′-AGAGCTTCAGGCAGGCAGTA-3′
SEQ ID NO: 26: IL-1β primer reverse: 5′-AGGTGCTCATGTCCTCATCC-3′
SEQ ID NO: 27: Mcp-1 primer forward: 5′-CCAGCAAGATGATCCCAATG-3′
SEQ ID NO: 28: Mcp-1 primer reverse: 5′-TTCTTGGGGTCAGCACAGAC-3′
SEQ ID NO: 29: Prkaa1 primer forward:
5′-GGTGTACGGAAGGCAAAATGGC-3′
SEQ ID NO: 30: Prkaa1 primer reverse:
5′-CAGGATTCTTCCTTCGTACACGC-3′
SEQ ID NO: 31: Prkaa2 primer forward:
5′-CTGAAGCCAGAGAATGTGCTGC-3′
SEQ ID NO: 32: Prkaa2 primer reverse:
5′-GAGATGACCTCAGGTGCTGCAT-3′
SEQ ID NO: 33: Prkab1 primer forward:
5′-CCAAAAGTGCTCCGATGTGTCTG-3′
SEQ ID NO: 34: Prkab1 primer reverse:
5′-GGGCTTTGAACCTCTCTTCTGG-3′
SEQ ID NO: 35: Prkab2 primer forward:
5′-GACTTCGTTGCCATCCTGGATC-3′
SEQ ID NO: 36: Prkab2 primer reverse:
5′-CCAAGCTGACTGGTAACCACAG-3′
SEQ ID NO: 37: Prkag1 primer forward:
5′-TCTCCGCCTTACCTGTAGTGGA-3′
SEQ ID NO: 38: Prkag1 primer reverse:
5′-GCAGGGCTTTTGTCACAGACAC-3′
SEQ ID NO: 39: Prkag2 primer forward:
5′-CTCCTCATCCAAAGAGTCTTCGC-3′
SEQ ID NO: 40: Prkag2 primer reverse:
5′-TGGGTGTTGACGGAGAAGAGGA-3′
SEQ ID NO: 41: Prkaig3 primer forward:
5′-AAGCGGCTACTCAAGTTCCTGC-3′
SEQ ID NO: 42: Prkag3 primer reverse:
5′-CCAGAACTACAGCCAAATCTCGG-3′
SEQ ID NO: 43: Gapdh primer forward:
5′-CATCACTGCCACCCAGAAGACTG-3′
SEQ ID NO: 44: Gadph primer reverse:
5′-ATGCCAGTGAGCTTCCCGTTCAG-3′
SEQ ID NO: 45: Bglap1 primer forward:
5′-GCAATAAGGTAGTGAACAGACTCC-3′
SEQ ID NO: 46: Bglap1 primer reverse:
5′-CCATAGATGCGTTTGTAGGCGG-3′
SEQ ID NO: 47: Bglap2 primer forward:
5′-GCAATAAGGTAGTGAACAGACTCC-3′
SEQ ID NO: 48: Bglap2 primer reverse:
5′-GCGTTTGTAGGCGGTCTTCAAG-3′
SEQ ID NO: 49: Got1(Ast) primer forward:
5′-TGCTACTGGGATGCGGAGAAGA-3′
SEQ ID NO: 50: Got1(Ast) primer reverse:
5′-TGCATGACAGCAGCGATCTGCT-3′
SEQ ID NO: 51: Gpt1(Ast) primer forward:
5′-CCACTCAGTCTCTAAGGGCTAC-3′
SEQ ID NO: 52: Gpt1(Ast) primer reverse:
5′-ACACAACCGCACGCTCATCAGT-3′
SEQ ID NO: 53: Ckb(Creatine) primer forward:
5′-GCTCATTGACGACCACTTCCTC-3′
SEQ ID NO: 54: Ckb(Creatine) primer reverse:
5′-CCTCCTCGTTAATCCACACCAG-3′
SEQ ID NO: 55: mTOR primer forward:
5′-AGAAGGGTCTCCAAGGACGACT-3′
SEQ ID NO: 56: mTOR primer reverse:
5′-GCAGGACACAAAGGCAGCATTG-3′
SEQ ID NO: 57: Bmp2 primer forward:
5′-AACACCGTGCGCAGCTTCCATC-3′
SEQ ID NO: 58: Bmp2 primer reverse:
5′-CGGAAGATCTGGAGTTCTGCAG-3′
SEQ ID NO: 59: Bmrp2 primer forward:
5′-AGAGACCCAAGTTCCCAGAAGC-3′
SEQ ID NO: 60: Bmpr2 primer reverse:
5′-TCTCCTCAGCACACTGTGCAGT-3′
SEQ ID NO: 61: Bmp7 primer forward:
5′-GGAGCGATTTGACAACGAGACC-3′
SEQ ID NO: 62: Bmp7 primer reverse:
5′-AGTGGTTGCTGGTGGCTGTGAT-3′
SEQ ID NO: 63: Tgfbr2primer forward:
5′-CCTACTCTGTCTGTGGATGACC-3′
SEQ ID NO: 64: Tgfbr2 primer reverse:
5′-GACATCCGTCGTCTTGAACGAC-3′
SEQ ID NO: 65: ALP primer forward: 5′-ATGGGATGGGTGTCTCCACA-3′
SEQ ID NO: 66: ALP primer reverse: 5′-CCACGAAGGGGAACTTGTC-3′
SEQ ID NO: 67: COL1A primer forward: 5′-ATGACTATGAGTGGGAAGCA-3′
SEQ ID NO: 68: COL1A primer reverse: 5′-TGGGTCCCTCTGTTACACTTT-3′
SEQ ID NO: 69: OPN primer forward: 5′-CTCAGGCCAGTTGCAGCC-3′
SEQ ID NO: 70: OPN primer reverse:
5′-CAAAAGCAAATCACTGCAATTCTC-3′
SEQ ID NO: 71: RUNX2 primer forward: 5′-CCTGAACTCTGCACCAAGTC-3′
SEQ ID NO: 72: RUNX2 primer reverse: 5′-GAGGTGGCAGTGTCATCATC-3′
SEQ ID NO: 73: GAPDH primer forward:
5′-GGCATCCACTGTGGTCATGAG-3′
SEQ ID NO: 74: GAPDH primer reverse: 5′-TGCACCACCAACTGCTTAGC-3′
SEQ ID NO: 75: 4EBP1 primer forward:
5′-CACCAGCCCTTCCAGTGATGAG-3′
SEQ ID NO: 76: 4EBP1 primer reverse:
5′-CCTTGGTAGTGCTCCACACGAT-3′
SEQ ID NO: 77: AKT1 primer forward:
5′-TGGACTACCTGCACTCGGAGAA-3′
SEQ ID NO: 78: AKT1 primer reverse:
5′-GTGCGGCAAAAGGTCTTCATGG-3′
SEQ ID NO: 79: S6K1 primer forward:
5′-TATTGGCAGCCCACGAACACCT-3′
SEQ ID NO: 80: S6K1 primer reverse:
5′-GTCACATCCATCTGCTCTATGCC-3′
SEQ ID NO: 81: HKUOT-S2 peptide: KTVSLPR
SEQ ID NO: 82: HKUOT-S2 peptide: KGNLLECDGGNTAQMMAR
SEQ ID NO: 83: HKUOT-S2 peptide: TKSSLPGQTK
SEQ ID NO: 84: HKUOT-S2 peptide: KEVSLPR
SEQ ID NO: 85: HKUOT-S2 peptide: IKITTYRQ
SEQ ID NO: 86: HKUOT-S2 peptide: AASECEEAGFSVCVEVNGR
SEQ ID NO: 87: HKUOT-S2 peptide: APSTYGGGLSVSSSR
SEQ ID NO: 88: HKUOT-S2 peptide: DDSITPTEDSIKR
SEQ ID NO: 89: HKUOT-S2 peptide:
DTANLFPQTSLSLFMKPDTAGTFDVECLTT-DHYTGGMKQK
SEQ ID NO: 90: HKUOT-S2 peptide: FKLLNYCIPK
SEQ ID NO: 91: HKUOT-S2 peptide: HAETSSGGQAASSHEQAR
SEQ ID NO: 92: HKUOT-S2 peptide:
QADLILTAGTVTMKMAPSLVRLYEQMAEPK
SEQ ID NO: 93: HKUOT-S2 peptide: RSKLGSHHIDR
SEQ ID NO: 94: HKUOT-S2 peptide: SISISVAR
SEQ ID NO: 95: HKUOT-S2 peptide: SLVNLGGSK
SEQ ID NO: 96: HKUOT-S2 peptide: TEDGSDPPSGDFLTEGGGVR
SEQ ID NO: 97: HKUOT-S2 peptide: VATVSLPR

### EMBODIMENTS

Embodiment 1. An embodiment comprising an amino acid sequence according to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or any combination thereof.

Embodiment 2. An embodiment comprising the protein of claim 1.

Embodiment 3. The embodiment of claim 2, further comprising at least one carrier or excipient.

Embodiment 4. The embodiment of claim 2, wherein the protein is HKUOT-S2.

Embodiment 5. The embodiment of claim 4, wherein HKUOT-S2 is 32 kDA.

Embodiment 6. An embodiment of osteogenesis comprising: administering an effective amount of a composition comprising a protein comprising an amino acid sequence according to any of SEQ ID NO: 81-97 or a sequence having at least 90% sequence identity to any of SEQ ID NO: 81-97, or a peptide according to any of SEQ ID NO: 81-97 or a sequence having at least 90% sequence identity to any of SEQ ID NO: 81-97 to a subject.

Embodiment 7. The embodiment of claim 6, further comprising increasing the expression of Ep4, Ptges, Cox2, Eno2, Calca, or any combination thereof in the subject.

Embodiment 8. The embodiment of claim 6, further comprising activating the mTOR/4E-BP1, AMPK, and BMP signaling pathways in the subject.

Embodiment 9. The embodiment of claim 8, further comprising increasing the expression of Prkaa1, Prkaa2, Prkab1, Prkab2, Prkag1, Prkag2, Prkag3 and Mtor, or any combination thereof in the subject.

Embodiment 10. The embodiment of claim 6, further comprising inhibiting ovariectomized (OVX)- and glucocorticoid-induced osteoporosis.

Embodiment 11. The embodiment of claim 6, wherein the protein or peptide is administered to the subject at a dose of about of about 0.1 mg kg⁻¹ to about 10 mg kg⁻¹ in the composition.

Embodiment 12. The embodiment of claim 11, wherein the dose is about 2.18 mg kg⁻¹.

Embodiment 13. The embodiment of claim 6, wherein the composition further comprises at least one carrier or excipient.

Embodiment 14. An embodiment of treating an inflammatory disease, a spinal cord injury, a liver and kidney disease, a cardiovascular disease, diabetes, a post-menopausal syndrome, infertility, or a hematopoietic disease or enhancing wound healing, comprising: administering an effective amount of a composition comprising a protein comprising an amino acid sequence according to any of SEQ ID NO: 81-97 or a sequence having at least 90% sequence identity to any of SEQ ID NO: 81-97, or a peptide according to any of SEQ ID NO: 81-97 or a sequence having at least 90% sequence identity to any of SEQ ID NO: 81-97 to a subject.

Embodiment 15. The embodiment of claim 14, further comprising increasing the expression of Ep4, Ptges, Cox2, Eno2, Calca, or any combination thereof in the subject.

Embodiment 16. The embodiment of claim 14, further comprising activating the mTOR/4E-BP1, AMPK, and/or BMP signaling pathways in the subject.

Embodiment 17. The embodiment of claim 16, further comprising increasing the expression of Prkaa1, Prkaa2, Prkab1, Prkab2, Prkag1, Prkag2, Prkag3 and Mtor, or any combination thereof in the subject.

Embodiment 18. The embodiment of claim 14, further comprising inhibiting ovariectomized (OVX)- and glucocorticoid-induced osteoporosis.

Embodiment 19. The embodiment of claim 14, wherein the protein or peptide is administered to the subject at a dose of about of about 0.1 mg kg⁻¹ to about 10 mg kg⁻¹ in the composition.

Embodiment 20. The embodiment of claim 11, wherein the dose is about 2.18 mg kg⁻¹.

Embodiment 21. The embodiment of claim 11, wherein the liver or kidney disease is hepatitis, hepatic cirrhosis, or nephropathies; the cardiovascular disease is hypertension or cardiomyopathy; the inflammatory disease is osteoarthritis; the hematopoietic disease is hemolytic anemia; and the post-menopausal syndrome is osteoporosis.

### REFERENCES

[1] G.B.D.F. Collaborators, Global, regional, and national burden of bone fractures in 204 countries and territories, 1990-2019: a systematic analysis from the Global Burden of Disease Study 2019, Lancet Healthy Longev 2(9) (2021) e580-e592.

[2] C. M. Court-Brown, B. Caesar, Epidemiology of adult fractures: A review, Injury 37(8) (2006) 691-7.

[3]S. Polinder, J. Haagsma, M. Panneman, A. Scholten, M. Brugmans, E. Van Beeck, The economic burden of injury: Health care and productivity costs of injuries in the Netherlands, Accid Anal Prev 93 (2016) 92-100.

[4] C. I. Hsieh, K. Zheng, C. Lin, L. Mei, L. Lu, W. Li, F. P. Chen, Y. Wang, X. Zhou, F. Wang, G. Xie, J. Xiao, S. Miao, C. F. Kuo, Automated bone mineral density prediction and fracture risk assessment using plain radiographs via deep learning, Nat Commun 12(1) (2021) 5472.

[5] O.T.G.f.F.o.O.G.fC.M.o.P.O.i.H. Kong, T. P. Ip, S. K. Cheung, T. C. Cheung, T. C. Choi, S. L. Chow, Y. Y. Ho, S. Y. Kan, W. C. Kung, K. K. Lee, K. L. Leung, Y. Y. Leung, S. T. Lo, C. T. Sy, Y. W. Wong, K. Osteoporosis Society of Hong, The Osteoporosis Society of Hong Kong (OSHK): 2013 OSHK guideline for clinical management of postmenopausal osteoporosis in Hong Kong, Hong Kong Med J 19 Suppl 2 (2013) 1-40.

[6] J. R. Perez, D. Kouroupis, D. J. Li, T. M. Best, L. Kaplan, D. Correa, Tissue Engineering and Cell-Based Therapies for Fractures and Bone Defects, Front Bioeng Biotechnol 6 (2018) 105.

[7] M. T. Corvol, K. Tahiri, A. Montembault, A. Daumard, J. F. Savouret, F. Rannou, [Cell therapy in cartilage repair: cellular and molecular bases], J Soc Biol 202(4) (2008) 313-21.

[8] A. Laurent, A. Porcello, P. G. Fernandez, A. Jeannerat, C. Peneveyre, P. Abdel-Sayed, C. Scaletta, N. Hirt-Burri, M. Michetti, A. de Buys Roessingh, W. Raffoul, E. Allemann, O. Jordan, L. A. Applegate, Combination of Hyaluronan and Lyophilized Progenitor Cell Derivatives: Stabilization of Functional Hydrogel Products for Therapeutic Management of Tendinous Tissue Disorders, Pharmaceutics 13(12) (2021).

[9] L. Lafuente-Gracia, E. Borgiani, G. Nasello, L. Geris, Towards in silico Models of the Inflammatory Response in Bone Fracture Healing, Front Bioeng Biotechnol 9 (2021) 703725.

[10] R. E. Tomlinson, M. J. Silva, Skeletal Blood Flow in Bone Repair and Maintenance, Bone Res 1(4) (2013) 311-22.

[11] C. A. Loynes, J. A. Lee, A. L. Robertson, M. J. Steel, F. Ellett, Y. Feng, B. D. Levy, M. K. B. Whyte, S. A. Renshaw, PGE2 production at sites of tissue injury promotes an anti-inflammatory neutrophil phenotype and determines the outcome of inflammation resolution in vivo, Sci Adv 4(9) (2018) eaar8320.

[12] T. A. Einhorn, L. C. Gerstenfeld, Fracture healing: mechanisms and interventions, Nat Rev Rheumatol 11(1) (2015) 45-54.

[13] M. Phimphilai, Z. Zhao, H. Boules, H. Roca, R. T. Franceschi, BMP signaling is required for RUNX2-dependent induction of the osteoblast phenotype, J Bone Miner Res 21(4) (2006) 637-46.

[14] W. Huang, S. Yang, J. Shao, Y. P. Li, Signaling and transcriptional regulation in osteoblast commitment and differentiation, Front Biosci 12 (2007) 3068-92.

[15] S. Maeda, M. Hayashi, S. Komiya, T. Imamura, K. Miyazono, Endogenous TGF-beta signaling suppresses maturation of osteoblastic mesenchymal cells, EMBO J 23(3) (2004) 552-63.

[16] C. Y. Tang, M. Wu, D. Zhao, D. Edwards, A. McVicar, Y. Luo, G. Zhu, Y. Wang, H. D. Zhou, W. Chen, Y. P. Li, Runx1 is a central regulator of osteogenesis for bone homeostasis by orchestrating BMP and WNT signaling pathways, PLoS Genet 17(1) (2021) e1009233.

[17] L. Claes, S. Recknagel, A. Ignatius, Fracture healing under healthy and inflammatory conditions, Nat Rev Rheumatol 8(3) (2012) 133-43.

[18] R. V. M. Groven, J. van Koll, M. Poeze, T. J. Blokhuis, M. van Griensven, miRNAs Related to Different Processes of Fracture Healing: An Integrative Overview, Front Surg 8 (2021) 786564.

[19] S. Ansari, K. Ito, S. Hofmann, Cell Sources for Human In vitro Bone Models, Curr Osteoporos Rep 19(1) (2021) 88-100.

[20] R. L. Shin, C. W. Lee, O. Y. Shen, H. Xu, O. K. Lee, The Crosstalk between Mesenchymal Stem Cells and Macrophages in Bone Regeneration: A Systematic Review, Stem Cells Int 2021 (2021) 8835156.

[21] L. Gong, Y. Zhao, Y. Zhang, Z. Ruan, The Macrophage Polarization Regulates MSC Osteoblast Differentiation in vitro, Ann Clin Lab Sci 46(1) (2016) 65-71.

[22] D. I. Cho, M. R. Kim, H. Y. Jeong, H. C. Jeong, M. H. Jeong, S. H. Yoon, Y. S. Kim, Y. Ahn, Mesenchymal stem cells reciprocally regulate the M1/M2 balance in mouse bone marrow-derived macrophages, Exp Mol Med 46 (2014) e70.

[23] L. Y. Lu, F. Loi, K. Nathan, T. H. Lin, J. Pajarinen, E. Gibon, A. Nabeshima, L. Cordova, E. Jamsen, Z. Yao, S. B. Goodman, Pro-inflammatory M1 macrophages promote Osteogenesis by mesenchymal stem cells via the COX-2-prostaglandin E2 pathway, J Orthop Res 35(11) (2017) 2378-2385.

[24] N. Zhang, C. W. Lo, T. Utsunomiya, M. Maruyama, E. Huang, C. Rhee, Q. Gao, Z. Yao, S. B. Goodman, PDGF-BB and IL-4 co-overexpression is a potential strategy to enhance mesenchymal stem cell-based bone regeneration, Stem Cell Res Ther 12(1) (2021) 40.

[25] F. Moreno Sancho, Y. Leira, M. Orlandi, J. Buti, W. V. Giannobile, F. D'Aiuto, Cell-Based Therapies for Alveolar Bone and Periodontal Regeneration: Concise Review, Stem Cells Transl Med 8(12) (2019) 1286-1295.

[26] C. Dou, N. Ding, C. Zhao, T. Hou, F. Kang, Z. Cao, C. Liu, Y. Bai, Q. Dai, Q. Ma, F. Luo, J. Xu, S. Dong, Estrogen Deficiency-Mediated M2 Macrophage Osteoclastogenesis Contributes to M1/M2 Ratio Alteration in Ovariectomized Osteoporotic Mice, J Bone Miner Res 33(5) (2018) 899-908.

[27] Y. Saxena, S. Routh, A. Mukhopadhaya, Immunoporosis: Role of Innate Immune Cells in Osteoporosis, Front Immunol 12 (2021) 687037.

[28] D. H. Yang, M. Y. Yang, The Role of Macrophage in the Pathogenesis of Osteoporosis, Int J Mol Sci 20(9) (2019).

[29] P. Aveline, A. Cesaro, M. Mazor, M. B. T, E. Lespessailles, H. Toumi, Cumulative Effects of Strontium Ranelate and Impact Exercise on Bone Mass in Ovariectomized Rats, Int J Mol Sci 22(6) (2021).

[30] L. Gani, N. Anthony, L. Dacay, P. Tan, L. R. Chong, T. F. King, Incidence of Atypical Femoral Fracture and Its Mortality in a Single Center in Singapore, JBMR Plus 5(8) (2021) e10515.

[31] H. Hagino, T. Sugimoto, S. Tanaka, K. Sasaki, T. Sone, T. Nakamura, S. Soen, S. Mori, A randomized, controlled trial of once-weekly teriparatide injection versus alendronate in patients at high risk of osteoporotic fracture: primary results of the Japanese Osteoporosis Intervention Trial-05, Osteoporos Int 32(11) (2021) 2301-2311.

[32] A. Corrado, E. R. Sanpaolo, S. Di Bello, F. P. Cantatore, Osteoblast as a target of anti-osteoporotic treatment, Postgrad Med 129(8) (2017) 858-865.

[33] C. Tohda, X. Yang, M. Matsui, Y. Inada, E. Kadomoto, S. Nakada, H. Watari, N. Shibahara, Diosgenin-Rich Yam Extract Enhances Cognitive Function: A Placebo-Controlled, Randomized, Double-Blind, Crossover Study of Healthy Adults, Nutrients 9(10) (2017).

[34] B. Padhan, D. Panda, Potential of Neglected and Underutilized Yams (Dioscorea spp.) for Improving Nutritional Security and Health Benefits, Front Pharmacol 11 (2020) 496.

[35] S. Kim, M. Y. Shin, K. H. Son, H. Y. Sohn, J. H. Lim, J. H. Lee, I. S. Kwun, Yam (Dioscorea batatas) Root and Bark Extracts Stimulate Osteoblast Mineralization by Increasing Ca and P Accumulation and Alkaline Phosphatase Activity, Prev Nutr Food Sci 19(3) (2014) 194-203.

[36] Y. W. Liu, J. C. Liu, C. Y. Huang, C. K. Wang, H. F. Shang, W. C. Hou, Effects of oral administration of yam tuber storage protein, dioscorin, to BALB/c mice for 21-days on immune responses, J Agric Food Chem 57(19) (2009) 9274-9.

[37] K. Y. Peng, L. Y. Horng, H. C. Sung, H. C. Huang, R. T. Wu, Antiosteoporotic Activity of Dioscorea alata L. cv. Phyto through Driving Mesenchymal Stem Cells Differentiation for Bone Formation, Evid Based Complement Alternat Med 2011 (2011) 712892.

[38] K. L. Wong, Y. M. Lai, K. W. Li, K. F. Lee, T. B. Ng, H. P. Cheung, Y. B. Zhang, L. Lao, R. N. Wong, P. C. Shaw, J. H. Wong, Z. J. Zhang, J. K. Lam, W. C. Ye, S. C. Sze, A Novel, Stable, Estradiol-Stimulating, Osteogenic Yam Protein with Potential for the Treatment of Menopausal Syndrome, Sci Rep 5 (2015) 10179.

[39] N. Setyawan, J. S. Maninang, S. Suzuki, Y. Fujii, Variation in the Physical and Functional Properties of Yam (Dioscorea spp.) Flour Produced by Different Processing Techniques, Foods 10(6) (2021).

[40] S. Kumar, G. Das, H. S. Shin, J. K. Patra, Dioscorea spp. (A Wild Edible Tuber): A Study on Its Ethnopharmacological Potential and Traditional Use by the Local People of Similipal Biosphere Reserve, India, Front Pharmacol 8 (2017) 52.

[41] P. Tiwwari, B. Kumar, M. Kaur, G. Kaur, H. Kaur, Phytochemical screening and Extraction: A Review INTERNATIONALE PHARMACEUTICA SCIENCIA 1(98-106) (2011).

[42] P. Hong, S. Koza, E. S. Bouvier, Size-Exclusion Chromatography for the Analysis of Protein Biotherapeutics and their Aggregates, J Liq Chromatogr Relat Technol 35(20) (2012) 2923-2950.

[43] M. Widbiller, C. Rothmaier, D. Saliter, M. Wolflick, A. Rosendahl, W. Buchalla, G. Schmalz, T. Spruss, K. M. Galler, Histology of human teeth: Standard and specific staining methods revisited, Arch Oral Biol 127 (2021) 105136.

[44] C. M. Lima, A. K. Lima, M. G. Melo, M. R. Serafini, D. L. Oliveira, E. B. de Almeida, R. S. Barreto, P. C. Nogueira, V. R. Moraes, E. R. Oliveira, R. L. de Albuquerque, Jr., L. J. Quintans-Junior, A. A. Araujo, Bioassay-guided evaluation of Dioscorea villosa—an acute and subchronic toxicity, antinociceptive and anti-inflammatory approach, BMC Complement Altern Med 13 (2013) 195.

[45] J. Loffler, F. A. Sass, S. Filter, A. Rose, A. Ellinghaus, G. N. Duda, A. Dienelt, Compromised Bone Healing in Aged Rats Is Associated With Impaired M2 Macrophage Function, Front Immunol 10 (2019) 2443.

[46] R. Huang, X. Wang, Y. Zhou, Y. Xiao, RANKL-induced M1 macrophages are involved in bone formation, Bone Res 5 (2017) 17019.

[47] J. Chen, F. Long, mTORC1 Signaling Promotes Osteoblast Differentiation from Preosteoblasts, PLoS One 10(6) (2015) e0130627.

[48] S. Fitter, M. P. Matthews, S. K. Martin, J. Xie, S. S. Ooi, C. R. Walkley, J. D. Codrington, M. A. Ruegg, M. N. Hall, C. G. Proud, S. Gronthos, A. C. W. Zannettino, mTORC1 Plays an Important Role in Skeletal Development by Controlling Preosteoblast Differentiation, Mol Cell Biol 37(7) (2017).

[49] B. Padhan, J. K. Nayak, D. Panda, Natural antioxidant potential of selected underutilized wild yams (Dioscorea spp.) for health benefit, J Food Sci Technol 57(6) (2020) 2370-2376.

[50] R. S. Conlan, L. A. Griffiths, J. A. Napier, P. R. Shewry, S. Mantell, C. Ainsworth, Isolation and characterisation of cDNA clones representing the genes encoding the major tuber storage protein (dioscorin) of yam (Dioscorea cayenensis Lam.), Plant Mol Biol 28(3) (1995) 369-80.

[51] J. P. Bonjour, Protein intake and bone health, Int J Vitam Nutr Res 81(2-3) (2011) 134-42.

[52] E. Tsiridis, P. V. Giannoudis, Transcriptomics and proteomics: advancing the understanding of genetic basis of fracture healing, Injury 37 Suppl 1 (2006) S13-9.

[53] A. Salhotra, H. N. Shah, B. Levi, M. T. Longaker, Mechanisms of bone development and repair, Nat Rev Mol Cell Biol 21(11) (2020) 696-711.

[54] J. Hu, H. Liao, Z. Ma, H. Chen, Z. Huang, Y. Zhang, M. Yu, Y. Chen, J. Xu, Focal Adhesion Kinase Signaling Mediated the Enhancement of Osteogenesis of Human Mesenchymal Stem Cells Induced by Extracorporeal Shockwave, Sci Rep 6 (2016) 20875.

[55] Y. G. Wang, X. H. Qu, Y. Yang, X. G. Han, L. Wang, H. Qiao, Q. M. Fan, T. T. Tang, K. R. Dai, AMPK promotes osteogenesis and inhibits adipogenesis through AMPK-Gfi 1-OPN axis, Cell Signal 28(9) (2016) 1270-82.

[56] H. B. Noor, N. A. Mou, L. Salem, M. F. A. Shimul, S. Biswas, R. Akther, S. Khan, S. Raihan, M. M. Mohib, M. A. T. Sagor, Anti-inflammatory Property of AMP-activated Protein Kinase, Antiinflamm Antiallergy Agents Med Chem 19(1) (2020) 2-41.

[57] Y. Cheng, L. Huang, Y. Wang, Q. Huo, Y. Shao, H. Bao, Z. Li, Y. Liu, X. Li, Strontium promotes osteogenic differentiation by activating autophagy via the the AMPK/mTOR signaling pathway in MC3T3E1 cells, Int J Mol Med 44(2) (2019) 652-660.

[58] H. Liu, X. Li, J. Lin, M. Lin, Morroniside promotes the osteogenesis by activating PI3K/Akt/mTOR signaling, Biosci Biotechnol Biochem 85(2) (2021) 332-339.

[59] P. N. Tasli, S. Aydin, M. E. Yalvac, F. Sahin, Bmp 2 and bmp 7 induce odonto- and osteogenesis of human tooth germ stem cells, Appl Biochem Biotechnol 172(6) (2014) 3016-25.

[60] A. Bandyopadhyay, K. Tsuji, K. Cox, B. D. Harfe, V. Rosen, C. J. Tabin, Genetic analysis of the roles of BMP2, BMP4, and BMP7 in limb patterning and skeletogenesis, PLoS Genet 2(12) (2006) e216.

[61] E. H. Alcantara, M. Y. Shin, H. Y. Sohn, Y. M. Park, T. Kim, J. H. Lim, H. J. Jeong, S. T. Kwon, I. S. Kwun, Diosgenin stimulates osteogenic activity by increasing bone matrix protein synthesis and bone-specific transcription factor Runx2 in osteoblastic MC3T3-E1 cells, J Nutr Biochem 22(11) (2011) 1055-63.

[62] Y. W. Liu, H. F. Shang, C. K. Wang, F. L. Hsu, W. C. Hou, Immunomodulatory activity of dioscorin, the storage protein of yam (Dioscorea alata cv. Tainong No. 1) tuber, Food Chem Toxicol 45(11) (2007) 2312-8.

[63] T. Li, Z. L. Liu, M. Xiao, Z. Z. Yang, M. Z. Peng, C. D. Li, X. J. Zhou, J. W. Wang, Impact of bone marrow mesenchymal stem cell immunomodulation on the osteogenic effects of laponite, Stem Cell Res Ther 9(1) (2018) 100.

[64] X. Qu, Z. Zhai, X. Liu, H. Li, Z. Ouyang, C. Wu, G. Liu, Q. Fan, T. Tang, A. Qin, K. Dai, Dioscin inhibits osteoclast differentiation and bone resorption though down-regulating the Akt signaling cascades, Biochem Biophys Res Commun 443(2) (2014) 658-65.

[65] C. Zhu, N. Bao, S. Chen, J. Zhao, Dioscin enhances osteoblastic cell differentiation and proliferation by inhibiting cell autophagy via the ASPP2/NF-kappabeta pathway, Mol Med Rep 16(4) (2017) 4922-4926.

[66] X. Chen, W. Chen, Z. M. Aung, W. Han, Y. Zhang, G. Chai, LY3023414 inhibits both osteogenesis and osteoclastogenesis through the PI3K/Akt/GSK3 signalling pathway, Bone Joint Res 10(4) (2021) 237-249.

[67] R. A. Saxton, D. M. Sabatini, mTOR Signaling in Growth, Metabolism, and Disease, Cell 168(6) (2017) 960-976.

[68] Iksen, S. Pothongsrisit, V. Pongrakhananon, Targeting the PI3K/AKT/mTOR Signaling Pathway in Lung Cancer: An Update Regarding Potential Drugs and Natural Products, Molecules 26(13) (2021).

[69] M. Jhanwar-Uniyal, J. V. Wainwright, A. L. Mohan, M. E. Tobias, R. Murali, C. D. Gandhi, M. H. Schmidt, Diverse signaling mechanisms of mTOR complexes: mTORC1 and mTORC2 in forming a formidable relationship, Adv Biol Regul 72 (2019) 51-62.

[70] J. Chen, F. Long, mTORC1 signaling controls mammalian skeletal growth through stimulation of protein synthesis, Development 141(14) (2014) 2848-54.

[71] J. Chen, N. Holguin, Y. Shi, M. J. Silva, F. Long, mTORC2 signaling promotes skeletal growth and bone formation in mice, J Bone Miner Res 30(2) (2015) 369-78.

[72] J. S. Kwon, S. W. Kim, D. Y. Kwon, S. H. Park, A. R. Son, J. H. Kim, M. S. Kim, in vivo osteogenic differentiation of human turbinate mesenchymal stem cells in an injectable in situ-forming hydrogel, Biomaterials 35(20) (2014) 5337-5346.

[73] C. A. Anosike, O. Obidoa, L. U. Ezeanyika, Membrane stabilization as a mechanism of the anti-inflammatory activity of methanol extract of garden egg (Solanum aethiopicum), Daru 20(1) (2012) 76.

[74] D. O'Neil, H. Glowatz, M. Schlumpberger, Ribosomal RNA depletion for efficient use of RNA-seq capacity, Curr Protoc Mol Biol Chapter 4 (2013) Unit 4 19.

[75] S. Lê, J. Josse, F. Husson, FactoMineR: An R Package for Multivariate Analysis, Journal of Statistical Software 25(1) (2008) 25(1), 1-18.

[76] F. J. Rios, R. M. Touyz, A. C. Montezano, Isolation and Differentiation of Murine Macrophages, Methods Mol Biol 1527 (2017) 297-309.

[78] Kubi, J. A., A. S. Brah, K. M. C. Cheung, Y. L. Lee, K. F. Lee, S. C. W. Sze, W. Qiao, and K. W. Yeung. 2023. ‘A new osteogenic protein isolated from Dioscorea opposita Thunb accelerates bone defect healing through the mTOR signaling axis’, Bioact Mater, 27: 429-46.

1. A protein comprising an amino acid sequence according to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, or any combination thereof.

2. A composition comprising the protein of claim 1.

3. The composition of claim 2, further comprising at least one carrier or excipient.

4. The composition of claim 2, wherein the protein is HKUOT-S2 with a molecular of 32 kDA.

5. (canceled)

6. A method of osteogenesis comprising: administering an effective amount of a composition comprising a protein comprising an amino acid sequence according to any of SEQ ID NO: 81-97 or a sequence having at least 90% sequence identity to any of SEQ ID NO: 81-97, or a peptide according to any of SEQ ID NO: 81-97 or a sequence having at least 90% sequence identity to any of SEQ ID NO: 81-97 to a subject.

7. The method of claim 6, further comprising increasing the expression of Ep4, Ptges, Cox2, Eno2, Calca, or any combination thereof in the subject.

8. The method of claim 6, further comprising activating the mTOR/4E-BP1, AMPK, and BMP signaling pathways in the subject.

9. The method of claim 8, further comprising increasing the expression of Prkaa1, Prkaa2, Prkab1, Prkab2, Prkag1, Prkag2, Prkag3 and Mtor, or any combination thereof in the subject.

10. The method of claim 6, further comprising inhibiting ovariectomized (OVX)- and glucocorticoid-induced osteoporosis.

11. The method of claim 6, wherein the protein or peptide is administered to the subject at a dose of about of about 0.1 mg kg⁻¹ to about 10 mg kg⁻¹ in the composition.

12. The method of claim 11, wherein the dose is about 2.18 mg kg⁻¹.

13. The method of claim 6, wherein the composition further comprises at least one carrier or excipient.

14. A method of treating an inflammatory disease, a spinal cord injury, a liver or kidney disease, a cardiovascular disease, diabetes, a post-menopausal syndrome, infertility, or a hematopoietic disease or enhancing wound healing, comprising: administering an effective amount of a composition comprising a protein comprising an amino acid sequence according to any of SEQ ID NO: 81-97 or a sequence having at least 90% sequence identity to any of SEQ ID NO: 81-97, or a peptide according to any of SEQ ID NO: 81-97 or a sequence having at least 90% sequence identity to any of SEQ ID NO: 81-97 to a subject.

15. The method of claim 14, further comprising increasing the expression of Ep4, Ptges, Cox2, Eno2, Calca, or any combination thereof in the subject.

16. The method of claim 14, further comprising activating the mTOR/4E-BP1, AMPK, and/or BMP signaling pathways in the subject.

17. The method of claim 16, further comprising increasing the expression of Prkaa1, Prkaa2, Prkab1, Prkab2, Prkag1, Prkag2, Prkag3 and Mtor, or any combination thereof in the subject.

18. The method of claim 14, further comprising inhibiting ovariectomized (OVX)- and glucocorticoid-induced osteoporosis.

19. The method of claim 14, wherein the protein or peptide is administered to the subject at a dose of about of about 0.1 mg kg⁻¹ to about 10 mg kg⁻¹ in the composition.

20. The method of claim 11, wherein the dose is about 2.18 mg kg⁻¹.

21. The method of claim 14, wherein the liver or kidney disease is hepatitis, hepatic cirrhosis, or nephropathies; the cardiovascular disease is hypertension or cardiomyopathy; the inflammatory disease is osteoarthritis; the hematopoietic disease is hemolytic anemia; and the post-menopausal syndrome is osteoporosis.
